(19) United States
(12) Patent Application Publication
(10) Pub. No.: US 2010/0178277 A1
(43) Pub. Date: Jul. 15, 2010

(54) METHODS AND COMPOSITIONS FOR STIMULATING CELLS

(75) Inventors: David T. Hung, Redwood City, CA (US); Andrew Asher Protter, Palo Alto, CA (US)

Correspondence Address: MORRISON & FOERSTER LLP, 755 PAGE MILL RD, PALO ALTO, CA, 94304-1018, US

(73) Assignee: MEDIVATION NEUROLOGY, INC., San Francisco, CA (US)

(21) Appl. No.: 12/602,090

(22) PCT Filed: May 23, 2008
(86) PCT No.: PCT/US2008/006667

§ 371 Date: Dec. 22, 2009

Related U.S. Application Data
(60) Provisional application No. 60/931,771, filed on May 25, 2007.

(51) Int. Cl. A61K 35/12; A61K 31/437; C12N 5/07; C12N 5/079; A61P 25/00

(52) U.S. Cl. 424/93.7; 514/292; 435/375; 435/377

(57) ABSTRACT

The invention provides compositions and methods for treating, preventing, delaying the onset, and/or delaying the development of a disease or condition for which the activation, differentiation, and/or proliferation of one or more cell types is beneficial. These compositions and methods include, for example, a hydrogenated pyrido[4,3-b]indole such as dimebon and/or a cell that has been incubated with a hydrogenated pyrido[4,3-b]indole such as dimebon. In some embodiments, the compositions and methods also include a growth factor and/or an anti-cell death compound. The invention also provides methods of activating a cell, promoting the differentiation of a cell, and/or promoting the proliferation of a cell by incubating the cell with one or more hydrogenated pyrido[4,3-b]indoles or pharmaceutically acceptable salts thereof. In some embodiments, the cell is also incubated with one or more growth factors and/or anti-cell death compounds.

### CROSS-REFERENCE TO RELATED APPLICATIONS

This application claims priority to U.S. Provisional Patent Application No. 60/931,771 filed May 25, 2007, which is incorporated herein by reference in its entirety.

### STATEMENT OF RIGHTS TO INVENTIONS MADE UNDER FEDERALLY SPONSORED RESEARCH

Not applicable.

### TECHNICAL FIELD

The present invention relates to compositions and methods for treating, preventing, delaying the onset, and/or delaying the development of a disease or condition for which the activation, differentiation, and/or proliferation of one or more cell types is beneficial by administering to an individual in need thereof an effective amount of any of: (1) a therapeutic compound or pharmaceutically acceptable salt thereof, (2) a combination of (i) a therapeutic compound or pharmaceutically acceptable salt thereof and (ii) a growth factor and/or an anti-cell death compound, (3) a cell that has been incubated with a therapeutic compound or pharmaceutically acceptable salt thereof (4) a combination of (i) a therapeutic compound or pharmaceutically acceptable salt thereof and (ii) a cell that has been incubated with a therapeutic compound or pharmaceutically acceptable salt thereof, (5) a combination of (i) a therapeutic compound or pharmaceutically acceptable salt thereof, (ii) a cell that has been incubated with a therapeutic compound or pharmaceutically acceptable salt thereof, and (iii) a growth factor and/or an anti-cell death compound, (6) a combination of (i) a therapeutic compound or pharmaceutically acceptable salt thereof and (ii) a cell (such as a cell that has not been incubated with a therapeutic compound or pharmaceutically acceptable salt thereof), or (7) a combination of (i) a therapeutic compound or pharmaceutically acceptable salt thereof, (ii) a cell (such as a cell that has not been incubated with a therapeutic compound or pharmaceutically acceptable salt thereof), and (iii) a growth factor and/or an anti-cell death compound. The above therapies may also be referred to herein as “therapies (1)-(7).” In some embodiments, both a growth factor and an anti-cell death compound are administered to the individual. In some variations, the therapeutic compound is dimebon.

The invention also provides methods of activating a cell, promoting the differentiation of a cell, and/or promoting the proliferation of a cell by incubating the cell with one or more therapeutic compounds or pharmaceutically acceptable salts thereof. In some embodiments, the cell is also incubated with one or more growth factors and/or anti-cell death compounds.

### BACKGROUND OF THE INVENTION

Numerous indications implicate cell death and/or decreased cell function and would benefit from the activation, differentiation, and/or proliferation of one or more cell types. For example, neuronal cell death is believed to be associated with various neuronal indications. For example, compounds and pharmaceutical compositions for treating and/or preventing neuronal and non-neuronal indications and methods of inhibiting neuronal cell death and/or enhancing survival of neurons are highly desired. In addition, compounds that increase the effectiveness of existing neurons would also have therapeutic value.

### Summary of Hydrogenated Pyrido[4,3-b]indoles

Known compounds of the class of tetra- and hexahydro-1H-pyrido[4,3-b]indole derivatives manifest a broad spectrum of biological activity. In the series of 2,3,4,5-tetrahydro-1H-pyrido[4,3-b]indoles the following types of activity have been found: antihistamine activity (DE 1,813,229, filed Dec. 6, 1968; DE 1,952,800, filed Oct. 20, 1969), central depressive and anti-inflammatory activity (U.S. Pat. No. 3,718,657, filed Dec. 3, 1970), neuroleptic activity (Herbert C. A., Plattner S. S., Welch W. M., Mol. Pharm. 1980, 17(1):38-42) and others. 2,3,4,4a,5,9b-hexahydro-1H-pyrido[4,3-b]indole derivatives show psychotropic (Welch W. M., Harbert C. A., Weissman A., Koe B. K., J. Med. Chem., 1986, 29(10):2093-2099), antiaggressive, antiarrhythmic and other types of activity.

Several drugs, such as diazoline (mebhydroline), dimebon, dorastine, carbidine (dicarbine), stobadine and gevotroline, based on tetra- or hexahydro-1H-pyrido[4,3-b]indole derivatives are known to have been manufactured. Diazoline (2-methyl-5-benzyl-2,3,4,5-tetrahydro-1H-pyrido[4,3-b]indole dihydrochloride) (Klyuev M. A., Drugs, used in “Medical Pract.”, USSR, Moscow, “Meditzina” Publishers, 1991, p. 512) and dimebon (2,8-dimethyl-5-(2-(6-methyl-3-pyridyl)ethyl-2,3,4,5-tetrahydro-1H-pyrido[4,3-b]indole dihydrochloride) (M. D. Mashkovsky, “Medicinal Drugs” in 2 vol. Vol. 1-12th Ed., Moscow, “Meditzina” Publishers, 1993, p. 383) as well as dorastine (2-methyl-8-chloro-5-[2-(6-methyl-3-pyridyl)ethyl]-2,3,4,5-tetrahydro-1H-pyrido[4,3-b]indole dihydrochloride) (USAN and USP dictionary of drugs names (United States Adopted Names, 1961-1988, current US Pharmacopoeia and National Formula for Drugs and other nonproprietary drug names), 1989, 26th Ed., p. 196) are known as antihistamine drugs; carbidine (dicarbine) (cis(±)-2,8-dimethyl-2,3,4,4a,5,9b-hexahydro-1H-pyrido[4,3-b]indole dihydrochloride) is a neuroleptic agent having an antidepressive effect (L. N. Yakhontov, R. G. Glushkov, Synthetic Drugs, ed. by A. G. Natradze, Moscow, “Meditzina” Publishers, 1983, p. 234-237), and its (−)isomer, stobadine, is known as an antiarrythmic agent (Kitlova M., Gibela P., Drimal J., Bratisl. Lek. Listy, 1985, vol. 84, No. 5, p. 542-549); gevotroline 8-fluoro-2-(3-(3-pyridyl)propyl)-2,3,4,5-tetrahydro-1H-pyrido[4,3-b]indole dihydrochloride is an antipsychotic and anxiolytic agent (Abou-Gharbi M., Patel U. R., Webb M. B., Moyer J. A., Ardnee T. H., J. Med. Chem., 1987, 30:1818-1823). Dimebon has been used in medicine as an antiallergic agent (Inventor's Certificate No. 1138164, IP Class A61K 31/47,5, C07 D 209/52, published on Feb. 7, 1985) in Russia for over 20 years.

As described in U.S. Pat. No. 6,187,785, hydrogenated pyrido[4,3-b]indole derivatives, such as dimebon, have NMDA antagonist properties, which make them useful for treating neurodegenerative diseases, such as Alzheimer's disease. See also U.S. Pat. No. 7,071,206. As described in WO 2005/055951, hydrogenated pyrido[4,3-b]indole derivatives, such as dimebon, are useful as human or veterinary geroprotectors e.g., by delaying the onset and/or development of an age-associated or related manifestation and/or pathology or condition, including disturbance in skin-hair integument, vision disturbance and weight loss. U.S. patent application Ser. No. 11/543,341, filed Oct. 4, 2006, and U.S. patent application Ser. No. 11/543,529, filed Oct. 4, 2006, disclose hydrogenated pyrido[4,3-b]indole derivatives, such as dimebon, as neuroprotectors for use in treating and/or preventing and/or slowing the progression or onset and/or development of Huntington's disease. See also Russian patent application filed Jan. 25, 2006 with an English language translated title of “Agent for Treatment of Schizophrenia Based on Hydrogenated Pyrido[4,3-b]indoles (Variations), a Pharmacological Agent Based on it, and a Method of Using it.”

### Significant Medical Need

There remains a significant interest in and need for additional or alternative therapies for treating, preventing, delaying the onset, and/or delaying the development of a disease or condition for which the activation, differentiation, and/or proliferation of one or more cell types is beneficial. Preferably, new therapies can improve the quality of life and/or prolong the survival time for individuals with a disease or condition for which the activation, differentiation, and/or proliferation of one or more cell types is beneficial.

### BRIEF SUMMARY OF THE INVENTION

The hydrogenated pyrido[4,3-b]indole dimebon was determined to stimulate neurite outgrowth and neurogenesis. Thus, dimebon functions as a growth factor and is expected to promote the activation, differentiation, and/or proliferation of a variety of cell types. This ability of dimebon to function as a small molecule growth factor is striking given that most growth factors are proteins that are much larger and have a much different three-dimensional structure than dimebon.

The present invention relates to compositions and methods for treating, preventing, delaying the onset, and/or delaying the development of a disease or condition for which the activation, differentiation, and/or proliferation of one or more cell types is beneficial, such as a neuronal indication, by administering to an individual in need thereof an effective amount of any of: (1) a therapeutic compound or pharmaceutically acceptable salt thereof, (2) a combination of (i) a therapeutic compound or pharmaceutically acceptable salt thereof and (ii) a growth factor and/or an anti-cell death compound, (3) a cell that has been incubated with a therapeutic compound or pharmaceutically acceptable salt thereof (4) a combination of (i) a therapeutic compound or pharmaceutically acceptable salt thereof and (ii) a cell that has been incubated with a therapeutic compound or pharmaceutically acceptable salt thereof, (5) a combination of (i) a therapeutic compound or pharmaceutically acceptable salt thereof, (ii) a cell that has been incubated with a therapeutic compound or pharmaceutically acceptable salt thereof, and (iii) a growth factor and/or an anti-cell death compound, (6) a combination of (i) a therapeutic compound or pharmaceutically acceptable salt thereof and (ii) a cell (such as a cell that has not been incubated with a therapeutic compound or pharmaceutically acceptable salt thereof), or (7) a combination of (i) a therapeutic compound or pharmaceutically acceptable salt thereof, (ii) a cell (such as a cell that has not been incubated with a therapeutic compound or pharmaceutically acceptable salt thereof), and (iii) a growth factor and/or an anti-cell death compound. In one variation, the method is a method of treating a disease or condition for which the activation, differentiation, and/or proliferation of one or more cell types is beneficial by administering to an individual in need thereof an effective amount of any of therapies (1)-(7) above. In another variation, the method is a method of preventing or slowing the onset and/or development of a disease or condition for which the activation, differentiation, and/or proliferation of one or more cell types is beneficial in an individual who has a mutated or abnormal gene associated with the disease or condition by administering to an individual in need thereof an effective amount of any of therapies (1)-(7) above. In another variation, the method is a method of slowing the progression of a disease or condition for which the activation, differentiation, and/or proliferation of one or more cell types is beneficial in an individual who has been diagnosed with the disease or condition by administering to an individual in need thereof an effective amount of any of therapies (1)-(7) above.

The invention also provides methods of activating a cell and/or promoting the differentiation of a cell and/or promoting the proliferation of a cell by incubating the cell with one or more therapeutic compounds or pharmaceutically acceptable salts thereof and/or one or more growth factors and/or anti-cell death compounds. Any of the methods described herein may include a step of selecting an individual (e.g., a human) who is in need of such therapy or is at risk for needing such therapy. In any method or other embodiment described herein, the compound may be the therapeutic compound dimebon or a pharmaceutically acceptable salt thereof, such as a hydrochloride salt or dihydrochloride salt thereof.

Pharmaceutical compositions are embraced, such as a pharmaceutical composition comprising (i) a therapeutic compound or pharmaceutically acceptable salt thereof in an amount sufficient to activate a cell, promote the differentiation of a cell, promote the proliferation of a cell, or any combination of two or more of the foregoing, and (ii) a pharmaceutically acceptable carrier. In another aspect, the invention provides a pharmaceutical composition comprising a combination of (i) a therapeutic compound or pharmaceutically acceptable salt thereof and (ii) a growth factor and/or an anti-cell death compound. In another aspect, the invention provides a pharmaceutical composition comprising a cell that has been incubated with a therapeutic compound or pharmaceutically acceptable salt thereof. In another aspect, the invention provides a pharmaceutical composition comprising a combination of (i) a therapeutic compound or pharmaceutically acceptable salt thereof and (ii) a cell that has been incubated with a therapeutic compound or pharmaceutically acceptable salt thereof. In another aspect, the invention provides a pharmaceutical composition comprising a combination of (i) a therapeutic compound or pharmaceutically acceptable salt thereof, (ii) a cell that has been incubated with a therapeutic compound or pharmaceutically acceptable salt thereof, and (iii) a growth factor and/or an anti-cell death compound. In another aspect, the invention provides a pharmaceutical composition comprising a combination of (i) a therapeutic compound or pharmaceutically acceptable salt thereof and (ii) a cell (such as a cell that has not been incubated with a therapeutic compound or pharmaceutically acceptable salt thereof). In another aspect, the invention provides a pharmaceutical composition comprising a combination of (i) a therapeutic compound or pharmaceutically acceptable salt thereof, (ii) a cell (such as a cell that has not been incubated with a therapeutic compound or pharmaceutically acceptable salt thereof), and (iii) a growth factor and/or an anti-cell death compound. In one variation, the pharmaceutical composition, such as any composition described above or here, further comprises a pharmaceutically acceptable carrier. The invention also provides that any of the compositions described may be for use as a medicament and/or for use in the manufacture of a medicament.

Kits comprising the therapies of the invention are also embraced, such as a kit (i) a therapeutic compound or pharmaceutically acceptable salt thereof in an amount sufficient to activate a cell, promote the differentiation of a cell, promote the proliferation of a cell, or any combination of two or more of the foregoing, and (ii) instructions for use in a disease or condition for which the activation, differentiation, and/or proliferation of one or more cell types is beneficial. In one aspect, the invention provides a kit comprising (i) a therapeutic compound or pharmaceutically acceptable salt thereof and (ii) a growth factor and/or an anti-cell death compound. In another aspect, the invention provides a kit comprising a cell that has been incubated with a therapeutic compound or pharmaceutically acceptable salt thereof. In another aspect, the invention provides a kit comprising (i) a therapeutic compound or pharmaceutically acceptable salt thereof and (ii) a cell that has been incubated with a therapeutic compound or pharmaceutically acceptable salt thereof. In another aspect, the invention provides a kit comprising (i) a therapeutic compound or pharmaceutically acceptable salt thereof, (ii) a cell that has been incubated with a therapeutic compound or pharmaceutically acceptable salt thereof, and (iii) a growth factor and/or an anti-cell death compound. In another aspect, the invention provides a kit comprising (i) a therapeutic compound or pharmaceutically acceptable salt thereof and (ii) a cell (such as a cell that has not been incubated with a therapeutic compound or pharmaceutically acceptable salt thereof). In another aspect, the invention provides a kit comprising (i) a therapeutic compound or pharmaceutically acceptable salt thereof, (ii) a cell (such as a cell that has not been incubated with a therapeutic compound or pharmaceutically acceptable salt thereof), and (iii) a growth factor and/or an anti-cell death compound. Any of the kits described herein, such as those above, may include directions for use in a disease or condition for which the activation, differentiation, and/or proliferation of one or more cell types is beneficial.

Other features and advantages of the invention will be apparent from the following detailed description and from the claims.

### BRIEF DESCRIPTION OF THE FIGURES

FIG. 1 is a dose response curve for neurite outgrowth in primary rat cortical neurons with a vehicle control and a positive control of brain derived neurotrophic factor (BDNF).

FIGS. 2A-2C are representative images of neurite outgrowth of cortical neurons treated with a vehicle control (FIG. 2A), 140 nM dimebon (FIG. 2B), or the positive control brain-derived neurotrophic factor (BDNF, brain-derived neurotrophic factor) (FIG. 2C).

FIG. 3 is a dose response curve for neurite outgrowth in primary rat hippocampal neurons with a vehicle control and a positive control of brain derived neurotrophic factor (BDNF).

FIG. 4 is a dose response curve for neurite outgrowth in primary rat spinal motor neurons with a vehicle control and a positive control of brain derived neurotrophic factor (BDNF).

FIGS. 5A and 5B illustrate the effect of Dimebon (100 nM) on neurite outgrowth using primary hippocampal neurons evaluated by measuring neurite length (expressed % of control, FIG. 5A) and number of neurites per neuron (FIG. 5B), respectively

FIGS. 6A and 6B are graphs of the number of total (FIG. 6A) and neuronal (FIG. 6B) hippocampal cells stained with BrdU after 14 days. FIG. 6A shows the number of BrdU IR positive cells in the hippocampus of rats treated with Dimebon at 10 mg/kg (group A), 30 mg/kg (group B), 60 mg/kg (group C) and with an equal volume of vehicle (saline; group D). FIG. 6B shows the number of cells positive for both NeuN (a marker specific for the neuronal lineage) and BrdU IR in the hippocampus of rats treated with Dimebon at 10 mg/kg (group A), 30 mg/kg (group B), 60 mg/kg (group C) and with an equal volume of vehicle (saline; group D). A significant increase in BrdU positive progenitor cells as well as BrdU positive neurons was detected between 60 mg/kg Dimebon and vehicle-treated groups. Data are represented by means+SEM. * . . . p=0.05.

FIGS. 7A and 7B are graphs of the number of total (FIG. 7A) or neuronal (FIG. 7B) dentate gyrus cells stained with BrdU after 14 days. FIG. 7A shows the number of BrdU IR positive cells in the dentate gyms of rats treated with Dimebon at 10 mg/kg (group A), 30 mg/kg (group B), 60 mg/kg (group C) and with an equal volume of vehicle (saline; group D). FIG. 7B shows the number cells positive for both NeuN (a marker specific for the neuronal lineage) and BrdU IR in the dentate gyms of rats treated with Dimebon at 10 mg/kg (group A), 30 mg/kg (group B), 60 mg/kg (group C), and with an equal volume of vehicle (saline; group D). A significant increase of BrdU positive progenitor cells as well as BrdU positive neurons was detected between 60 mg/kg Dimebon and vehicle-treated groups as well as for progenitors versus vehicle after 30 mg/kg Dimebon treatment. Data are represented by means+SEM. * . . . p=0.05.

### DETAILED DESCRIPTION OF THE INVENTION

### Definitions

Unless clearly indicated otherwise, use of the terms “a”, “an” and the like refers to one or more.

The term “about” as used herein refers to the usual error range for the respective value readily known to the skilled person in this technical field. Reference to “about” a value or parameter herein includes (and describes) embodiments that are directed to that value or parameter per se.

Unless clearly indicated otherwise, “an individual” as used herein intends a mammal, including but not limited to human, bovine, primate, equine, canine, feline, porcine, and ovine animals. Thus, the invention finds use in both human medicine and in the veterinary context, including use in agricultural animals and domestic pets. The individual may be a human who has been diagnosed with or is suspected of having a disease or condition for which the activation, differentiation, and/or proliferation of one or more cell types is beneficial. The disease or condition may be a neuronal indication or a non-neuronal indication. The disease or condition may involve neurodegeneration or degenerative disorders or trauma relating to non-neuronal indications. The individual may be a human who exhibits one or more symptoms associated with a neuronal indication. The individual may be a human who has a mutated or abnormal gene associated with a disease or condition for which the activation, differentiation, and/or proliferation of one or more cell types is beneficial. The individual may be a human who is genetically or otherwise predisposed to developing a disease or condition for which the activation, differentiation, and/or proliferation of one or more cell types is beneficial.

As used herein, “treatment” or “treating” is an approach for obtaining a beneficial or desired result, including clinical results. For purposes of this invention, beneficial or desired results include, but are not limited to: alleviation of a symptom and/or diminishment of the extent of a symptom and/or preventing a worsening of a symptom associated with a disease or condition for which the activation, differentiation, and/or proliferation of one or more cell types is beneficial, including but not limited to: a neurodegenerative disease; Alzheimer's disease, age-associated hair loss, age-associated weight loss, age-associated vision disturbance, Huntington's disease and related polyglutamine expansion diseases, schizophrenia, canine cognitive dysfunction syndrome (CCDS), neuronal death mediated ocular disease, macular degeneration, amyotrophic lateral sclerosis (ALS), multiple sclerosis, Parkinson's disease, Lewy body disease, Menkes disease, Wilson disease, Creutzfeldt-Jakob disease, Fahr disease, acute or chronic disorders involving cerebral circulation, such as stroke, ischemic brain injury or cerebral hemorrhagic insult, age-associated memory impairment (AAMI), or mild cognitive impairment (MCI). For example, beneficial or desired results for treating Alzheimer's disease include, but are not limited to, one or more of the following: inhibiting or suppressing the formation of amyloid plaques, reducing, removing, or clearing amyloid plaques, improving cognition or reversing cognitive decline, sequestering soluble Aβ peptide circulating in biological fluids, reducing Aβ peptide (including soluble and deposited) in a tissue (e.g., the brain), inhibiting and/or reducing accumulation of Aβ peptide in the brain, inhibiting and/or reducing toxic effects of Aβ peptide in a tissue (e.g., the brain), decreasing one more symptoms resulting from the disease (e.g., abnormalities of memory, problem solving, language, calculation, visuospatial perception, judgment and/or behavior), increasing the quality of life, decreasing the dose of one or more other medications required to treat the disease, delaying the progression of the disease, and/or prolonging survival of the individual. Preferably, treatment of a disease or condition for which the activation, differentiation, and/or proliferation of one or more cell types is beneficial with a therapeutic compound or a pharmaceutically acceptable salt thereof, such as dimebon, is accompanied by no or fewer side effects than are associated with currently available therapies and/or improves the quality of life of the individual. The invention embraces treating, preventing, delaying the onset, and/or delaying the development of a disease or condition that is believed to or does involve cell death, cell injury, cell loss, impaired or decreased cell function, impaired or decreased cell proliferation, or impaired or decreased cell differentiation, where the cell may be any specific cell type described herein, such as a non-neuronal cell. Accordingly, one aspect of the invention is treating a disease that implicates a non-neuronal cell, such as treatment of degenerative disorders or trauma relating to non-neuronal cells, cardiac muscle cells for the treatment of heart disease, pancreatic islet cells for the treatment of diabetes, adipocytes for the treatment of anorexia or wasting associated with many diseases including AIDS, cancer, and cancer treatments, including chemotherapy, smooth muscle cells to be used in vascular grafts and intestinal grafts, cartilage to be used to treat cartilage injuries and degenerative conditions of cartilage and osteoarthritis, and replace cells damaged or lost to bacterial or viral infection, or those lost to traumatic injuries such as burns, fractures, and lacerations.

As used herein, “delaying” development of a disease or condition means to defer, hinder, slow, retard, stabilize and/or postpone development of the disease or condition. This delay can be of varying lengths of time, depending on the history of the disease and/or individual being treated. As is evident to one skilled in the art, a sufficient or significant delay can, in effect, encompass prevention, in that the individual does not develop the disease or condition. For example, a method that “delays” development of Alzheimer's disease is a method that reduces probability of disease development in a given time frame and/or reduces extent of the disease in a given time frame, when compared to not using the method. Such comparisons are typically based on clinical studies, using a statistically significant number of subjects. For example, Alzheimer's disease development can be detected using standard clinical techniques, such as routine neurological examination, patient interview, neuroimaging, detecting alterations of levels of specific proteins in the serum or cerebrospinal fluid (e.g., amyloid peptides and Tau), computerized tomography (CT) or magnetic resonance imaging (MRI). Similar techniques are known in the art for other diseases and conditions. Development may also refer to disease progression that may be initially undetectable and includes occurrence, recurrence and onset.

As used herein, an “at risk” individual is an individual who is at risk of developing a disease or condition for which the activation, differentiation, and/or proliferation of one or more cell types is beneficial. An individual “at risk” may or may not have a detectable disease or condition, and may or may not have displayed detectable disease prior to the treatment methods described herein. “At risk” denotes that an individual has one or more so-called risk factors, which are measurable parameters that correlate with development of a disease or condition and are known in the art. An individual having one or more of these risk factors has a higher probability of developing the disease or condition than an individual without these risk factor(s). These risk factors include, but are not limited to, age, sex, race, diet, history of previous disease, presence of precursor disease, genetic (i.e., hereditary) considerations, and environmental exposure. For example, individuals at risk for Alzheimer's disease include, e.g., those having relatives who have experienced this disease, and those whose risk is determined by analysis of genetic or biochemical markers. Genetic markers of risk for Alzheimer's disease include mutations in the APP gene, particularly mutations at position 717 and positions 670 and 671 referred to as the Hardy and Swedish mutations, respectively (Hardy, Trends Neurosci., 20:154-9, 1997). Other markers of risk are mutations in the presenilin genes (e.g., PS1 or PS2), ApoE4 alleles, family history of Alzheimer's disease, hypercholesterolemia and/or atherosclerosis. Other such factors are known in the art for other diseases and conditions.

As used herein, the term “non-neuronal indications” or refers to and intends diseases or conditions that are believed to involve, or be associated with, or do involve or are associated with non-neuronal cell death and/or impaired non-neuronal function or decreased non-neuronal function or a disease or condition involving degenerative disorders or trauma relating to non-neuronal cells. Examples of non-neuronal cells include, but are not limited to, a skin cell, a hematopoietic cell, a smooth muscle cell, a cardiac cell, a cardiac muscle cell, a skeletal muscle cell, a bone cell, a cartilage cell, a pancreatic cell or an adipocyte.

As used herein, the term “neuronal indications” refers to and intends diseases or conditions that are believed to involve, or be associated with, or do involve or are associated with neuronal cell death and/or impaired neuronal function or decreased neuronal function.

As used herein, the term “neuron” represents a cell of ectodermal embryonic origin derived from any part of the nervous system of an animal. Neurons express well-characterized neuron-specific markers, including neurofilament proteins, NeuN (Neuronal Nuclei marker), MAP2, and class III tubulin. Included as neurons are, for example, hippocampal, cortical, midbrain dopaminergic, spinal motor, sensory, sympathetic, septal cholinergic, and cerebellar neurons.

As used herein, the term “neurite outgrowth” or “neurite activation” refers to the extension of existing neuronal processes (i.e., axons and dendrites) and the growth or sprouting of new neuronal processes (i.e., axons and dendrites). Neurite outgrowth or neurite activation may alter neural connectivity, resulting in the establishment of new synapses or the remodeling of existing syapses.

As used herein, the term “neurogenesis” refers to the generation of new nerve cells from undifferentiated neuronal progenitor cells, also known as multipotential neuronal stem cells. Neurogenesis actively produces new neurons, astrocytes, glia, Schwann cells, oligodendrocytes and other neural lineages. Much neurogenesis occurs early in human development, though it continues later in life, particularly in certain localized regions of the adult brain. Multipotential neuronal stem cells, the self-renewing, multipotent cells that generate the main phenotypes of the nervous system, have been isolated from various areas of the adult brain, including the hippocampus, the dentate gyrus, and the subventricular zone, and have also been isolated from areas not normally associated with neurogenesis, such as the spinal cord.

As used herein, the term “neural connectivity” refers to the number, type, and quality of connections (“synapses”) between neurons in an organism. Synapses form between neurons, between neurons and muscles (a “neuromuscular junction”), and between neurons and other biological structures, including internal organs, endocrine glands, and the like. Synapses are specialized structures by which neurons transmit chemical or electrical signals to each other and to non-neuronal cells, muscles, tissues, and organs. Compounds that affect neural connectivity may do so by establishing new synapses (e.g., by neurite outgrowth or neurite activation) or by altering or remodeling existing synapses. Synaptic remodeling refers to changes in the quality, intensity or type of signal transmitted at particular synapses.

As used herein, the term “neuropathy” refers to a disorder characterized by altered function and structure of motor, sensory, and autonomic neurons of the nervous system, initiated or caused by a primary lesion or other dysfunction of the nervous system. The four cardinal patterns of peripheral neuropathy are polyneuropathy, mononeuropathy, mononeuritis multiplex and autonomic neuropathy. The most common form is (symmetrical) peripheral polyneuropathy, which mainly affects the feet and legs. A radiculopathy involves spinal nerve roots, but if peripheral nerves are also involved the term radiculoneuropathy is used. The form of neuropathy may be further broken down by cause, or the size of predominant fiber involvement, i.e. large fiber or small fiber peripheral neuropathy. Central neuropathic pain can occur in spinal cord injury, multiple sclerosis, and some strokes, as well as fibromyalgia. Neuropathy may be associated with varying combinations of weakness, autonomic changes and sensory changes. Loss of muscle bulk or fasciculations, a particular fine twitching of muscle may be seen. Sensory symptoms encompass loss of sensation and “positive” phenomena including pain. Neuropathies are associated with a variety of disorders, including diabetes (i.e., diabetic neuropathy), fibromyalgia, multiple sclerosis, and herpes zoster infection, as well as with spinal cord injury and other types of nerve damage.

As used herein, the term “schizophrenia” includes all forms and classifications of schizophrenia known in the art, including, but not limited to catatonic type, hebephrenic type, disorganized type, paranoid type, residual type or undifferentiated type schizophrenia and deficit syndrome and/or those described in American Psychiatric Association: Diagnostic and Statistical Manual of Mental Disorders, Fourth Edition, Washington D.C., 2000 or in International Statistical Classification of Diseases and Related Health Problems, or otherwise known to those of skill in the art.

As used herein “geroprotective activity” or “geroprotector” means a biological activity that slows down ageing and/or prolongs life and/or increases or improves the quality of life via a decrease in the amount and/or the level of intensity of pathologies or conditions that are not life-threatening but are associated with the aging process and which are typical for elderly people. Pathologies or conditions that are not life-threatening but are associated with the aging process include such pathologies or conditions as loss of sight (cataract), deterioration of the dermatohairy integument (alopecia), and an age-associated decrease in weight due to the death of muscular and/or fatty cells.

As used herein, unless clearly indicated otherwise, the term “treatment of CCDS” or “treating CCDS” means controlling (improving or preventing a worsening of) one or more clinical symptoms associated with CCDS, recognizing that the duration and magnitude of response may vary with individual canines.

“Neuronal death mediated ocular disease” intends an ocular disease in which death of the neuron is implicated in whole or in part. The disease may involve death of photoreceptors. The disease may involve retinal cell death. The disease may involve ocular nerve death by apoptosis. Particular neuronal death mediated ocular diseases include but are not limited to macular degeneration, glaucoma, retinitis pigmentosa, congenital stationary night blindness (Oguchi disease), childhood onset severe retinal dystrophy, Leber congenital amaurosis, Bardet-Biedle syndrome, Usher syndrome, blindness from an optic neuropathy, Leber's hereditary optic neuropathy, color blindness and Hansen-Larson-Berg syndrome.

As used herein, the term “macular degeneration” includes all forms and classifications of macular degeneration known in the art, including, but not limited to diseases that are characterized by a progressive loss of central vision associated with abnormalities of Bruch's membrane, the choroid, the neural retina and/or the retinal pigment epithelium. The term thus encompasses disorders such as age-related macular degeneration (ARMD) as well as rarer, earlier-onset dystrophies that in some cases can be detected in the first decade of life. Other maculopathies include North Carolina macular dystrophy, Sorsby's fundus dystrophy, Stargardt's disease, pattern dystrophy, Best disease, and Malattia Leventinese.

“Amyotrophic lateral sclerosis” or “ALS” are terms understood in the art and are used herein to denote a progressive neurodegenerative disease that affects upper motor neurons (motor neurons in the brain) and/or lower motor neurons (motor neurons in the spinal cord) and results in motor neuron death. As used herein, the term “ALS” includes all of the classifications of ALS known in the art, including, but not limited to classical ALS (typically affecting both lower and upper motor neurons), Primary Lateral Sclerosis (PLS, typically affecting only the upper motor neurons), Progressive Bulbar Palsy (PBP or Bulbar Onset, a version of ALS that typically begins with difficulties swallowing, chewing and speaking), Progressive Muscular Atrophy (PMA, typically affecting only the lower motor neurons) and familial ALS (a genetic version of ALS).

The term “Parkinson's disease” is understood in the art and as used herein refers to any medical condition wherein an individual experiences one or more symptom associated with Parkinson's disease, such as without limitation one or more of the following symptoms: rest tremor, cogwheel rigidity, bradykinesia, postural reflex impairment, good response to 1-dopa treatment, the absence of prominent oculomotor palsy, cerebellar or pyramidal signs, amyotrophy, dyspraxia and/or dysphasia. In a specific embodiment, the present invention is utilized for the treatment of a dopaminergic dysfunction-related disorder. In a specific embodiment, the individual with Parkinson's disease has a mutation or polymorphism in a synuclein, parkin or NURR1 nucleic acid that is associated with Parkinson's disease. In one embodiment, the individual with Parkinson's disease has defective or decreased expression of a nucleic acid or a mutation in a nucleic acid that regulates the development and/or survival of dopaminergic neurons.

As used herein, the term “mild cognitive impairment” or “MCI” refers to a type of cognitive disorder characterized by a more pronounced deterioration in cognitive functions than is typical for normal age-related decline. As a result, elderly or aged patients with MCI have greater than normal difficulty performing complex daily tasks and learning, but without the inability to perform normal social, everyday, and/or professional functions typical of patients with Alzheimer's disease, or other similar neurodegenerative disorders eventually resulting in dementia. MCI is characterized by subtle, clinically manifest deficits in cognition, memory, and functioning, amongst other impairments, which are not of sufficient magnitude to fulfill criteria for diagnosis of Alzheimer's disease or other dementia. MCI also encompasses injury-related MCI, defined herein as cognitive impairment resulting from certain types of injury, such as nerve injury (i.e., battlefield injuries, including post-concussion syndrome, and the like), neurotoxic treatment (i.e., adjuvant chemotherapy resulting in “chemo brain” and the like), and tissue damage resulting from physical injury or other neurodegeneration, which is separate and distinct from mild cognitive impairment resulting from stroke, ischemia, hemorrhagic insult, blunt force trauma, and the like.

As used herein, the term “age-associated memory impairment” or “AAMI” refers to a condition that may be identified as GDS stage 2 on the global deterioration scale (GDS) (Reisberg, et al. (1982) Am. J. Psychiatry 139: 1136-1139) which differentiates the aging process and progressive degenerative dementia in seven major stages. The first stage of the GDS is one in which individuals at any age have neither subjective complaints of cognitive impairment nor objective evidence of impairment. These GDS stage 1 individuals are considered normal. The second stage of the GDS applies to those generally elderly persons who complain of memory and cognitive functioning difficulties such as not recalling names as well as they could five or ten years previously or not recalling where they have placed things as well as they could five or ten years previously. These subjective complaints appear to be very common in otherwise normal elderly individuals. AAMI refers to persons in GDS stage 2, who may differ neurophysiologically from elderly persons who are normal and free of subjective complaints, i.e., GDS stage 1. For example, AAMI subjects have been found to have more electrophysiologic slowing on a computer analyzed EEG than GDS stage 1 elderly persons (Prichep, John, Ferris, Reisberg, et al. (1994) Neurobiol. Aging 15: 85-90).

As used herein, the term “autism” refers to a brain development disorder that impairs social interaction and communication and causes restricted and repetitive behavior, typically appearing during infancy or early childhood. The cognitive and behavioral defects are thought to result in part from altered neural connectivity. Autism encompasses related disorders sometimes referred to as “autism spectrum disorder,” as well as Asperger syndrome and Rett syndrome.

As used herein, the term “nerve injury” or “nerve damage” refers to physical damage to nerves, such as avulsion injury (i.e., where a nerve or nerves have been torn or ripped) or spinal cord injury (i.e., damage to white matter or myelinated fiber tracts that carry sensation and motor signals to and from the brain). Spinal cord injury can occur from many causes, including physical trauma (i.e., car accidents, sports injuries, and the like), tumors impinging on the spinal column, developmental disorders, such as spina bifida, and the like.

As used herein, the term “myasthenia gravis” refers to a non-cognitive neuromuscular disorder caused by immune-mediated loss of acetylcholine receptors at neuromuscular junctions of skeletal muscle. Clinically, MG typically appears first as occasional muscle weakness in approximately two-thirds of patients, most commonly in the extraocular muscles. These initial symptoms eventually worsen, producing drooping eyelids (ptosis) and/or double vision (diplopia), often causing the patient to seek medical attention. Eventually, many patients develop general muscular weakness that may fluctuate weekly, daily, or even more frequently. Generalized MG often affects muscles that control facial expression, chewing, talking, swallowing, and breathing; before recent advances in treatment, respiratory failure was the most common cause of death.

As used herein, the term “Guillain-Barré syndrome” refers to a non-cognitive disorder in which the body's immune system attacks part of the peripheral nervous system. The first symptoms of this disorder include varying degrees of weakness or tingling sensations in the legs. In many instances the weakness and abnormal sensations spread to the arms and upper body. These symptoms can increase in intensity until certain muscles cannot be used at all and, when severe, the patient is almost totally paralyzed. In these cases the disorder is life threatening—potentially interfering with breathing and, at times, with blood pressure or heart rate—and is considered a medical emergency. Most patients, however, recover from even the most severe cases of Guillain-Barré syndrome, although some continue to have a certain degree of weakness.

As used herein, the term “multiple sclerosis” or “MS” refers to an autoimmune condition in which the immune system attacks the central nervous system (CNS), leading to demyelination of neurons. It may cause numerous symptoms, many of which are non-cognitive, and often progresses to physical disability. MS affects the areas of the brain and spinal cord known as the white matter. White matter cells carry signals between the grey matter areas, where the processing is done, and the rest of the body. More specifically, MS destroys oligodendrocytes which are the cells responsible for creating and maintaining a fatty layer, known as the myelin sheath, which helps the neurons carry electrical signals. MS results in a thinning or complete loss of myelin and, less frequently, the cutting (transection) of the neuron's extensions or axons. When the myelin is lost, the neurons can no longer effectively conduct their electrical signals. Almost any neurological symptom can accompany the disease. MS takes several forms, with new symptoms occurring either in discrete attacks (relapsing forms) or slowly accumulating over time (progressive forms). Most people are first diagnosed with relapsing-remitting MS but develop secondary-progressive MS (SPMS) after a number of years. Between attacks, symptoms may go away completely, but permanent neurological problems often persist, especially as the disease advances.

As used herein, by “growth factor” is meant a compound that stimulates cellular proliferation, cellular differentiation, and/or cell survival. Examples of growth factors include vascular endothelial cell growth factors, trophic growth factors, NT-3, NT-4/5, hepatocyte growth factor (HGF), ciliary neurotrophic factor (CNTF), transforming growth factor alpha (TGF-alpha), TGF-beta family members, myostatin (GDF-8), neurotrophin-3, platelet-derived growth factor (PDGF), GDNF (glial-derived neurotrophic factor), epidermal growth factor (EGF) family members, insulin-like growth factor (IGF), insulin, bone morphogenic proteins (BMPs), erythropoietin, thrombopoietin, Wnts, hedgehogs, heregulins, fragments thereof, and mimics thereof. Examples of other growth factors are described herein.

As used herein, by “vascular endothelial cell growth factor (VEGF)” is meant a VEGF protein, fragment or mimic thereof, such as any protein that results from alternate splicing of mRNA from a single, 8 exon, VEGF gene or homolog thereof. The different VEGF splice variants are referred to by the number of amino acids they contain. In humans, the isoforms are VEGF121, VEGF145, VEGF165, VEGF189 and VEGF206; the rodent orthologs of these proteins contain one less amino acid. These proteins differ by the presence or absence of short C-terminal domains encoded by exons 6a, 6b and 7 of the VEGF gene. These domains have important functional consequences for the VEGF splice variants as they mediate interactions with heparan sulfate proteoglycans and neuropilin co-receptors on the cell surface, enhancing their ability to bind and activate the VEGF signaling receptors. VEGF exerts neuroprotective effects via its cell surface receptor Flk-1. Flk-1 activates PI3 kinase/AKT and ERK to exert a neuroprotective effect (Matsuzaki et al., “Vascular endothelial growth factor rescues hippocampal neurons from glutamate-induced toxicity: signal transduction cascades,” FASEB J., 2001 May; 15(7):1218-20). In various embodiments, the amino acid sequence of the VEGF protein or protein fragment is at least or about 50%, 60%, 70%, 80%, 90%, 95% or 100% identical to that of the corresponding region of a human VEGF protein. In some embodiments, the VEGF fragment contains at least 25, 50, 75, 100, 150 or 200 contiguous amino acids from a full-length VEGF protein and has at least or about 5%, 10%, 20%, 30%, 40%, 50%, 60%, 70%, 80%, 90% or 100% of an activity of a corresponding full-length VEGF protein.

As used herein, by “trophic growth factor” is meant a growth factor that inhibits or prevents cell death, promotes cell survival, and/or enhances cell function (e.g., neurite outgrowth or neurogenesis). Exemplary trophic growth factors include IGF-1, fibroblast growth factor (FGF), nerve growth factor (NGF), brain-derived neurotrophic factor (BDNF), granulocyte colony stimulating factor (G-CSF), granulocyte-macrocyte colony stimulating factor (GM-CSF), neurotrophin-3, glial derived neurotrophic factor (GDNF), epidermal growth factor (EGF) or TGFα and mimics and fragments thereof. In various embodiments, the amino acid sequence of a trophic growth factor or fragment thereof is at least 50%, 60%, 70%, 80%, 90%, 95% or 100% identical to that of the corresponding region of a human growth factor. In some embodiments, the growth factor fragment contains at least 25, 50, 75, 100, 150 or 200 contiguous amino acids from a full-length growth factor and has at least or about 5%, 10%, 20%, 30%, 40%, 50%, 60%, 70%, 80%, 90% or 100% of an activity of a corresponding full-length growth factor. Examples of other trophic growth factors are described herein.

As used herein, by “anti-cell death compound” is meant a compound that reduces or eliminates cell death. In some embodiments, the compound reduces cell death (e.g., neuronal cell death in the brain or a region of the brain or non-neuronal cell death) by at least or about 10%, 20%, 30%, 40%, 50%, 60%, 70%, 80%, 90%, 95% or 100% as compared to the corresponding cell death in the same subject prior to treatment or compared to the corresponding cell death in other subjects not receiving the combination therapy. Exemplary anti-cell death compounds include anti-apoptotic compounds, such as IAP proteins, Bcl-2 proteins, Bcl-X_{L}, Trk receptors, Akt, PI3 kinase, Gab, Mek, E1B55K, Raf, Ras, PKC, PLC, FRS2, rAPs/SH2B, Np73, fragments thereof, and mimics thereof.

As used herein, by “anti-apoptotic compound” is meant a compound that reduces or eliminates programmed cell death. In some embodiments, the compound reduces programmed cell death (e.g., neuronal cell death in the brain or a region of the brain or non-neuronal cell death) by at least or about 10%, 20%, 30%, 40%, 50%, 60%, 70%, 80%, 90%, 95% or 100% as compared to the corresponding programmed cell death in the same subject prior to treatment or compared to the corresponding programmed cell death in other subjects not receiving the compound. Exemplary anti-apoptotic compounds include IAP proteins, Bcl-2 proteins, Bcl-X_{L}, Trk receptors, Akt, PI3 kinase, Gab, Mek, E1B55K, Raf, Ras, PKC, PLC, FRS2, rAPs/SH2B, Np73, fragments thereof, and mimics thereof.

As used herein, by “therapeutic compound” is meant any compound disclosed herein under the “Therapeutic Compound” heading, including any pharmaceutically acceptable salt thereof. In one variation, the therapeutic compound is dimebon.

As used herein, by “combination therapy” is meant a therapy that includes two or more different pharmaceutically active compounds or cells. Exemplary combination therapies include (1) a combination of (i) a therapeutic compound or pharmaceutically acceptable salt thereof and (ii) a growth factor and/or an anti-cell death compound, (2) a combination of (i) a therapeutic compound or pharmaceutically acceptable salt thereof and (ii) a cell that has been incubated with a therapeutic compound or pharmaceutically acceptable salt thereof, (3) a combination of (i) a therapeutic compound or pharmaceutically acceptable salt thereof, (ii) a cell that has been incubated with a therapeutic compound or pharmaceutically acceptable salt thereof, and (iii) a growth factor and/or an anti-cell death compound, (4) a combination of (i) a therapeutic compound or pharmaceutically acceptable salt thereof and (ii) a cell (such as a cell that has not been incubated with a therapeutic compound or pharmaceutically acceptable salt thereof), and (5) a combination of (i) a therapeutic compound or pharmaceutically acceptable salt thereof, (ii) a cell (such as a cell that has not been incubated with a therapeutic compound or pharmaceutically acceptable salt thereof), and (iii) a growth factor and/or an anti-cell death compound. In some embodiments, both a growth factor and an anti-cell death compound are included in the combination therapy. In some variations, the therapeutic compound is dimebon. In some variations, the combination therapy optionally includes one or more pharmaceutically acceptable carriers or excipients, non-pharmaceutically active compounds, and/or inert substances.

As used herein, by “pharmaceutically active compound,” “pharmacologically active compound” or “active ingredient” is meant a chemical compound that induces a desired effect, e.g., treating and/or preventing and/or delaying the onset and/or the development of Alzheimer's disease.

As used herein, the term “effective amount” intends such amount of a compound or therapy (e.g., a therapeutic compound, a growth factor, anti-cell death compound or a cell) which in combination with its parameters of efficacy and toxicity, as well as based on the knowledge of the practicing specialist should be effective in a given therapeutic form. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. The compounds and/or therapies in a combination therapy of the invention may be administered sequentially, simultaneously, or continuously using the same or different routes of administration for each compound. Thus, an effective amount of a combination therapy includes an amount of the first therapy and an amount of the second or subsequent therapy that, when administered sequentially, simultaneously, or continuously, produces a desired outcome. Suitable doses of any of the coadministered compounds may optionally be lowered due to the combined action (e.g., additive or synergistic effects) of the compounds.

In various embodiments, treatment with the combination of a first and a second or subsequent therapy may result in an additive or even synergistic (e.g., greater than additive) result compared to administration of either therapy alone. In some embodiments, a lower amount of each compound is used as part of a combination therapy compared to the amount generally used for individual therapy. Preferably, the same or greater therapeutic benefit is achieved using a combination therapy than by using any of the individual compounds alone. In some embodiments, the same or greater therapeutic benefit is achieved using a smaller amount (e.g., a lower dose or a less frequent dosing schedule) of a compound in a combination therapy than the amount generally used for individual therapy. Preferably, the use of a small amount of compound results in a reduction in the number, severity, frequency, or duration of one or more side-effects associated with the compound.

The term “simultaneous administration,” as used herein, means that a first therapy and a second or subsequent therapy in a combination therapy are administered with a time separation of no more than about 15 minutes, such as no more than about any of 10, 5, or 1 minutes. When the therapies are administered simultaneously, the first and second therapies may be contained in the same composition (e.g., a composition comprising both a therapeutic compound and a growth factor and/or an anti-cell death compound) or in separate compositions (e.g., a therapeutic compound is contained in one composition and a growth factor and/or an anti-cell death compound is contained in another composition). The invention embraces methods for the simultaneous administration of a combination of (i) a therapeutic compound or pharmaceutically acceptable salt thereof and (ii) a growth factor and/or an anti-cell death compound. Also embraced are methods for the simultaneous administration of (i) a therapeutic compound or pharmaceutically acceptable salt thereof and (ii) a cell that has been incubated with a therapeutic compound or pharmaceutically acceptable salt thereof. Also embraced are methods for the simultaneous administration of (i) a therapeutic compound or pharmaceutically acceptable salt thereof, (ii) a cell that has been incubated with a therapeutic compound or pharmaceutically acceptable salt thereof, and (iii) a growth factor and/or an anti-cell death compound. Also embraced are methods for the simultaneous administration of (i) a therapeutic compound or pharmaceutically acceptable salt thereof and (ii) a cell (such as a cell that has not been incubated with a therapeutic compound or pharmaceutically acceptable salt thereof). Also embraced are methods for the simultaneous administration of (i) a therapeutic compound or pharmaceutically acceptable salt thereof, (ii) a cell (such as a cell that has not been incubated with a therapeutic compound or pharmaceutically acceptable salt thereof), and (iii) a growth factor and/or an anti-cell death compound.

As used herein, the term “sequential administration” means that the first therapy and second therapy in a combination therapy are administered with a time separation of more than about 15 minutes, such as more than about any of 20, 30, 40, 50, or 60 minutes, or more than about any of 1 hour to about 24 hours, about 1 hour to about 48 hours, about 1 day to about 7 days, about 1 week to about 4 weeks, about 1 week to about 8 weeks, about 1 week to about 12 weeks, about 1 month to about 3 months, or about 1 month to about 6 months. Either the first therapy or the second therapy may be administered first. The first and second therapies are contained in separate compositions, which may be contained in the same or different packages or kits. The invention embraces the sequential administration of all combinations described herein, such as those described in the preceding paragraph.

As used herein, “unit dosage form” refers to physically discrete units, suitable as unit dosages, each unit containing a predetermined quantity of active ingredient calculated to produce the desired therapeutic effect in association with the required pharmaceutical carrier.

As used herein, the term “controlled release” refers to a drug-containing formulation or fraction thereof in which release of the drug is not immediate, i.e., with a “controlled release” formulation, administration does not result in immediate release of the drug into an absorption pool. The term encompasses depot formulations designed to gradually release the drug compound over an extended period of time. Controlled release formulations can include a wide variety of drug delivery systems, generally involving mixing the drug compound with carriers, polymers or other compounds having the desired release characteristics (i.e., pH-dependent or non-pH-dependent solubility, different degrees of water solubility, and the like) and formulating the mixture according to the desired route of delivery (i.e., coated capsules, implantable reservoirs, injectable solutions containing biodegradable capsules, and the like).

For use herein, unless clearly indicated otherwise, the term “sustained release system” (also referred to as “a system” or “the system”) refers to a drug delivery system capable of sustaining the rate of delivery of a compound to an individual for a desired duration, which may be an extended duration. A desired duration may be any duration that is longer than the time required for a corresponding immediate-release dosage form to release the same amount (e.g., by weight or by moles) of compound, and can be hours or days. A desired duration may be at least the drug elimination half life of the administered compound and may be about any of, e.g., at least about 6 hours, or at least about 12 hours, or at least about 24 hours, or at least about 30 hours, or at least about 48 hours, or at least about 72 hours, or at least about 96 hours, or at least about 120 hours, or at least about 144 or more hours, and can be at least about one week, at least about 2 weeks, at least about 3 weeks, at least about 4 weeks, at least about 8 weeks, at least about 16 weeks or more.

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

As used herein, the term “purified cell” means a cell that has been separated from one or more components that are present when the cell is produced. In some embodiments, the cell is at least about 60%, by weight, free from other components that are present when the cell is produced. In various embodiments, the cell is at least about 75%, 90%, or 99%, by weight, pure. A purified cell can be obtained, for example, by purification (e.g., extraction) from a natural source, fluorescence-activated cell-sorting, or other techniques known to the skilled artisan. Purity can be assayed by any appropriate method, such as fluorescence-activated cell-sorting. In some embodiments, the purified cell is incorporated into a pharmaceutical composition of the invention or used in a method of the invention. The pharmaceutical composition of the invention may have additives, carriers, or other components in addition to the purified cell.

By “multipotential stem cell” or “MSC” is meant a cell that (i) has the potential of differentiating into at least two cell types and (ii) exhibits self-renewal, meaning that at a cell division, at least one of the two daughter cells will also be a stem cell. The non-stem cell progeny of a single MSC are capable of differentiating into multiple cell types. For example, non-stem cell progeny of neuronal stem cells are capable of differentiating into neurons, astrocytes, Schwann cells, and oligodendrocytes. Similarly, non-stem cell progeny of non-neuronal stem cells have the potential to differentiate into other cell types, including non-neuronal cell types (e.g., a skin cell, a hematopoietic cell, a smooth muscle cell, a cardiac muscle cell, a skeletal muscle cell, a bone cell, a cartilage cell, a pancreatic cell or an adipocyte). Hence, the stem cell is “multipotent” because its progeny have multiple differentiative pathways.

### Overview of the Methods

The invention provides methods for treating, preventing, delaying the onset, and/or delaying the development of a disease or condition for which the activation, differentiation, and/or proliferation of one or more cell types is beneficial. Exemplary diseases and conditions include diseases and conditions that are believed to involve or be associated with, or do involve or are associated with, one or more of the following: cell death, cell injury, cell loss, impaired or decreased cell function, impaired or decreased cell proliferation, or impaired or decreased cell differentiation, where the cell may be any cell type, including the specific cell types described herein. The disease or condition may be one in which the activation, differentiation, and/or proliferation of cells such as neuronal stem cells or neurons or non-neuronal cells is expected to be or is beneficial. Some exemplary cell types include any stem cell (such as any self-renewing, multipotential cell). Other exemplary cell types, such as but not limited to those described under the heading “Exemplary Cells and Methods” may be modulated using the therapies and methods of the invention are described herein. Accordingly, the invention embraces treating, preventing, delaying the onset, and/or delaying the development of a disease or condition that is believed to or does involve cell death, cell injury, cell loss, impaired or decreased cell function, impaired or decreased cell proliferation, or impaired or decreased cell differentiation, where the cell may be any specific cell type described herein.

The invention also provides methods of activating a cell, promoting the differentiation of a cell, and/or promoting the proliferation of a cell by incubating the cell with one or more therapeutic compounds or pharmaceutically acceptable salts thereof. In some embodiments, the cell is also incubated with one or more growth factors and/or anti-cell death compounds.

The present invention is based in part on the striking discovery that dimebon (a representative hydrogenated pyrido[4,3-b]indole) functions as a small molecule growth factor. As described further below, dimebon stimulates neuronal outgrowth and neurogenesis (see, Examples 1 and 2). Simulating the activity, differentiation, and/or proliferation of neuronal cells ex vivo or in vivo is useful for the treatment of neurological conditions. In addition, hydrogenated pyrido[4,3-b]indoles and pharmaceutically acceptable salts thereof are expected to also be useful for promoting the activity, differentiation, and/or proliferation of non-neuronal cells. Accordingly, hydrogenated pyrido[4,3-b]indoles (or pharmaceutically acceptable salts thereof) or cells incubated with hydrogenated pyrido[4,3-b]indoles (or pharmaceutically acceptable salts thereof) can be used to treat any disease or condition for which the activation, differentiation, and/or proliferation of one or more cell types is beneficial.

### Methods for Activating Cells

Accordingly, the invention provides methods of activating a cell by incubating the cell with one or more hydrogenated pyrido[4,3-b]indoles or pharmaceutically acceptable salts thereof under conditions sufficient to activate the cell. For example, a therapeutic compound can be used to activate neurons by stimulating neurite outgrowth. As illustrated in Example 1, incubation of neurons with dimebon increased the length of axons from cortical neurons, hippocampal neurons, and spinal motor neurons. Based on the activation of neuronal cells with dimebon, dimebon is also expected to activate other cell types, such as any of the cell types described herein, including non-neuronal cells. Some exemplary cell types include any stem cell (such as any self-renewing, multipotential cell).

In various embodiments for the ex vivo incubation of cells with a therapeutic compound, a therapeutic compound such as dimebon in saline is added to cells at a concentration ranging from about 1 pM to about 5 mM, from about 10 pM to about 500 μM, from about 50 pM to about 100 μM, from about 0.25 nM to about 20 μM, from about 1 nM to about 5 μM, from about 6 nM to about 800 nM, from about 30 nM to about 160 nM. In various embodiments for the ex vivo incubation of cells with a therapeutic compound, a therapeutic compound such as dimebon in saline is added to cells at a concentration of about 0.01 nM, 0.05 nM, 0.25 nM, 1.25 nM, 6.25 nM, 31.25 nM, 156.25 nM, 781 nM, 3.905 μM, 19.530 μnM, 97.660 μM, or 488.280 μM.

In some embodiments, the cell is also incubated with a growth factor (e.g., a VEGF protein or a trophic growth factor) and/or an anti-cell death compound. The cell can be incubated with a therapeutic compound before, during, or after it is incubated with a growth factor and/or an anti-cell death compound. In some embodiments, incubation with a growth factor and/or an anti-cell death compound produces an additive or synergistic effect compared to incubation with a therapeutic compound alone. In some embodiments, the cell is incubated with both a growth factor and an anti-cell death compound.

In various embodiments, the incubation occurs ex vivo or in vivo. In some embodiments, a therapeutic compound is administered to an individual (such as an individual in need of one or more cell types) to activate a cell (e.g., a neuronal stem cell or a neuronal cell or a non-neuronal cell) in vivo. In some embodiments, a growth factor and/or an anti-cell death compound is administered to the individual to enhance the activation of a cell (e.g., a neuronal stem cell or a neuronal cell or non-neuronal cell) in vivo. In some embodiments, a dose of a therapeutic compound is administered orally, intravenously, intraperitoneally, subcutaneously, intrathecally, intramuscularly, intraocularly, transdermally, or topically (i.e., as eye drops or ear drops). In some embodiments, a dose of a therapeutic compound is administered once daily, twice daily, three times daily, or at higher frequencies. In some embodiments, a dose of a therapeutic composition is administered once a week, twice a week, three times a week, four times a week, or at higher frequencies. In some embodiments, a dose of a therapeutic compound is administered as a controlled release formulation every week, every two weeks, every three weeks, every four weeks, every five weeks, every six weeks, or at even longer intervals. In some embodiments, a dose (e.g., a dose for oral administration) of about 1 ng/day, 10 ng/day, 100 ng/day, 250 ng/day, 500 ng/day, 1 μg/day, 5 μg/day, 10 μg/day, 20 μg/day, 40 μg/day, 80 μg/day, 160 μg/day, 320 μg/day, or 120 mg/day of a therapeutic compound is administered. In some embodiments, the therapeutic compound is administered directly by infusion to the brain (e.g., intrathecal or intraventricular administration) at a dose of about 1 ng/day, 10 ng/day, 100 ng/day, 250 ng/day, 500 ng/day, 1 μg/day, 5 μg/day, 10 μg/day, 20 μg/day, 25 μg/day, 40 μg/day, 80 μg/day, 125 μg/day, 160 μg/day, 320 μg/day, or 120 mg/day. In some embodiments, a slow release pump or other device in the brain issued to administer any of the doses described herein. In some variations, the therapeutic hydrogenated pyrido[4,3-b]indole or pharmaceutically acceptable salt thereof is dimebon.

Cells that have been activated by incubation with a therapeutic compound (and optionally with a growth factor and/or an anti-cell death compound) are useful in any of the methods, compositions, and kits of the invention. In some embodiments, the cell is a neuron with axons that are at least about 5%, 10%, 20%, 30%, 40%, 50%, 60%, 70%, 80%, 90%, 100% longer (i) than the axons prior to incubation of the cell or (ii) than the axons of the corresponding control cell that was incubated under the same conditions without a therapeutic compound, growth factor, or anti-cell death compound.

Methods for Promoting the Differentiation and/or Proliferation of Cells

The invention also features methods of promoting the differentiation and/or proliferation of a cell by incubating a cell with a hydrogenated pyrido[4,3-b]indole or pharmaceutically acceptable salt thereof under conditions sufficient to promoting the differentiation and/or proliferation of the cell. As illustrated in Example 2, dimebon increased the number of dividing neurons in the rat hippocampus. Thus, dimebon may stimulate differentiation of neuronal stem cell into differentiated neuronal cells and/or stimulate the proliferation of neuronal stem cells or neuronal cells. Based on the increase in the number of neuronal cells due to administration of dimebon, dimebon is also expected to promote the differentiation and/or proliferation of other cell types, such as any of the cell types described herein. Some exemplary cell types include any multipotential stem cell (such as any self-renewing, multipotential cell).

In various embodiments for the ex vivo incubation of cells with a therapeutic compound, a therapeutic compound such as dimebon in saline is added to cells at a concentration ranging from about 1 pM to about 5 mM, from about 10 pM to about 500 μM, from about 50 pM to about 100 μM, from about 0.25 nM to about 20 μM, from about 1 nM to about 5 μM, from about 6 nM to about 800 nM, from about 30 nM to about 160 nM. In various embodiments for the ex vivo incubation of cells with a therapeutic compound, a therapeutic compound such as dimebon in saline is added to cells at a concentration of about 0.01 nM, 0.05 nM, 0.25 nM, 1.25 nM, 6.25 nM, 31.25 nM, 156.25 nM, 781 nM, 3.905 μM, 19.530 μM, 97.660 μM, or 488.280 μM.

In some embodiments, the cell is also incubated with a growth factor (e.g., a VEGF protein or a trophic growth factor) and/or an anti-cell death compound. The cell can be incubated with a therapeutic compound before, during, or after it is incubated with a growth factor and/or an anti-cell death compound. In some embodiments, incubation with a growth factor and/or an anti-cell death compound produces an additive or synergistic effect compared to incubation with a therapeutic compound alone.

In various embodiments, the incubation occurs ex vivo or in vivo. In some embodiments, a therapeutic compound is administered to an individual (such as an individual in need of one or more cell types) to promote the differentiation and/or proliferation of a cell (e.g., a neuronal stem cell or a neuronal cell or a non-neuronal cell) in vivo. In some embodiments, a growth factor and/or an anti-cell death compound is administered to the individual to enhance the differentiation and/or proliferation of a cell (e.g., a neuronal stem cell or a neuronal cell or non-neuronal cell) in vivo. In some embodiments, a dose of a therapeutic compound is administered orally, intravenously, intraperitoneally, subcutaneously, intrathecally, intramuscularly, intraocularly, transdermally, or topically (i.e., as eye drops or ear drops). In some embodiments, a dose of a therapeutic compound is administered once daily, twice daily, three times daily, or at higher frequencies. In some embodiments, a dose of a therapeutic composition is administered once a week, twice a week, three times a week, four times a week, or at higher frequencies. In some embodiments, a dose of a therapeutic compound is administered as a controlled release formulation every week, every two weeks, every three weeks, every four weeks, every five weeks, every six weeks, or at even longer intervals. In some embodiments, a dose (e.g., a dose for oral administration) of about 1 ng/day, 10 ng/day, 100 ng/day, 250 ng/day, 500 ng/day, 1 μg/day, 5 μg/day, 10 μg/day, 20 μg/day, 25 μg/day, 40 μg/day, 80 μg/day, 125 μg/day, 160 μg/day, 320 μg/day, or 120 mg/day of a therapeutic compound is administered. In some embodiments, the therapeutic compound is administered directly by infusion to the brain (e.g., intrathecal or intraventricular administration) at a dose of about 1 ng/day, 10 ng/day, 100 ng/day, 250 ng/day, 500 ng/day, 1 μg/day, 5 μg/day, 10 μg/day, 20 μg/day, 25 μg/day, 40 μg/day, 80 μg/day, 125 μg/day, 160 μg/day, 320 μg/day, or 120 mg/day. In some embodiments, a slow release pump or other device in the brain issued to administer any of the doses described herein. In some variations, the therapeutic hydrogenated pyrido[4,3-b]indole or pharmaceutically acceptable salt thereof is dimebon.

Accordingly, in one aspect, the invention provides a method of promoting the differentiation and/or proliferation of a cell comprising incubating a cell with a hydrogenated pyrido[4,3-b]indole or a pharmaceutically acceptable salt thereof under conditions sufficient to promote the differentiation and/or proliferation of the cell. In one embodiment, the differentiation and/or proliferation comprises neurite outgrowth and/or neurogenesis of the cell. In one embodiment, the differentiation and/or proliferation comprises neurite outgrowth. In one embodiment, the differentiation and/or proliferation comprises neurogenesis. In one embodiment, the hydrogenated pyrido[4,3-b]indole or pharmaceutically acceptable salt thereof is dimebon. In one embodiment, the method further comprises incubating the cell with a growth factor and/or an anti-cell death compound. In one embodiment, the cell type is selected from the group consisting of multipotential stem cells, neuronal stem cells, non-neuronal cell and neurons. In one embodiment, the cell type is a neuron, and the method increases the length of one or more axons of the neuron. In one embodiment, the cell type is a neuronal stem cell, and the method promotes the differentiation of the neuronal stem cell into a neuron. In one embodiment, the neuronal stem cell differentiates into a hippocampal neuron, cortical neuron, or spinal motor neuron. In one embodiment, the non-neuronal stem cell differentiates into a skin cell, a cardiac muscle cell, a skeletal muscle cell, a liver cell a kidney cell, or a cartilage cell. In one embodiment, the incubation occurs ex vivo. In one embodiment, the incubation occurs in vivo.

In another aspect, the invention provides a method of stimulating neurite outgrowth and/or enhancing neurogenesis of a cell comprising incubating a cell with a hydrogenated pyrido[4,3-b]indole or pharmaceutically acceptable salt thereof under conditions sufficient to stimulate neurite outgrowth and/or to enhance neurogenesis of the cell. In one embodiment, the hydrogenated pyrido[4,3-b]indole or pharmaceutically acceptable salt thereof is dimebon. In one embodiment, the method further comprises incubating the cell with a growth factor and/or an anti-cell death compound. In one embodiment, the cell type is selected from the group consisting of multipotential stem cells, neuronal stem cells, non-neuronal cell and neurons. In one embodiment, the cell type is a neuron, and the method increases the length of one or more axons of the neuron. In one embodiment, the cell type is a neuronal stem cell, and the method promotes the differentiation of the neuronal stem cell into a neuron. In one embodiment, the neuronal stem cell differentiates into a hippocampal neuron, cortical neuron, or spinal motor neuron. In one embodiment, the incubation occurs ex vivo. In one embodiment, the incubation occurs in vivo.

Cells that have been incubated with a therapeutic compound (and optionally with a growth factor and/or an anti-cell death compound) to promote their differentiation and/or proliferation are useful in any of the methods, compositions, and kits of the invention. In some embodiments, the number of cells increase by at least about 5%, 10%, 20%, 30%, 40%, 50%, 60%, 70%, 80%, 90%, 100%, 150%, 200%, compared to (i) the number of cell(s) prior to incubation or (ii) the number of cells generated from the same number of starting control cell(s) that were incubated under the same conditions without a therapeutic compound, growth factor, or anti-cell death compound.

### Methods for Differentiating Multipotential Stem Cells

In certain aspects, the invention features methods for differentiating multipotential stem cells (MSCs) by isolating MSCs from an individual, culturing the isolated MSCs in vitro, incubating the cultured MSCs with an amount of a hydrogenated pyrido[4,3-b]indole or pharmaceutically acceptable salt thereof effective to induce the multipotential stem cells to differentiate, and selecting the desired differentiated cell type from culture. In one embodiment, the method comprises incubating a multipotential stem cell isolated from an individual with an amount of a hydrogenated pyrido[4,3-b]indole or pharmaceutically acceptable salt thereof effective to induce the multipotential stem cells to differentiate. In certain embodiments, the MSCs differentiate into cortical neurons, hippocampal neurons, or spinal motor neurons. In certain embodiments, the hydrogenated pyrido[4,3-b]indole or pharmaceutically acceptable salt thereof is dimebon. MSCs are cells that have the potential to differentiate into at least two different cell types and divide asymmetrically, meaning that at each cell division, at least one of the two progeny cells produced will also be a multipotential stem cell.

In certain embodiments, MSCs are isolated from adult human or fetal tissues, including the umbilical cord. MSCs can be isolated from various regions of the brain, including the hippocampus, the dentate gyrus, and the subventricular region. MSCs can also be isolated from deep layers of the skin, bone marrow or plasma. Where MSCs are isolated as part of a complex biological mixture, such as bone marrow, plasma, or other tissue samples, additional purification steps may be required. MSCs may be separated from differentiated cells and other biological materials by any standard method known to one of ordinary skill in the art, such as flow cytometry, density gradient centrifugation, and the like.

After isolation from adult human or fetal tissues, MSCs are washed and triturated if necessary, then suspended in appropriate culture medium (i.e., Neurobasal medium (GIBCO)) to the desired concentration and placed in an appropriate culture vessel containing the suitable culture medium. The culture medium can be supplemented with factors that promote cell growth as desired, including, for example, serum-free culture supplements such as B27 (GIBCO), L-glutamine (GIBCO), growth factors and the like. In certain embodiments, the MSCs can be cultured in supplemented or unsupplemented medium in the absence of other cell types. In certain embodiments, the MSCs can be co-cultured with differentiated cell types from the same or a different developmental context. For example, neuronal MSCs obtained from the hippocampus can be cultured with differentiated neurons, oligodendrocytes, glial cells, or Schwann cells. Cells can be grown in a variety of culture vessels depending on the desired quantity and application, including flasks or wells on poly-L-lysine-coated plates, under standard conditions, such as 37° C. in 5% CO₂-95% air atmosphere. Once the MSCs have adhered to the plates and are growing normally, they can be treated with a therapeutic hydrogenated pyrido[4,3-b]indole or pharmaceutically acceptable salt thereof, such as dimebon in saline, at a concentration sufficient to induce differentiation. In one variation, the cells may also be treated with a growth factor and/or an anti-cell death compound.

In certain embodiments, the MSCs are induced to differentiate into specific cell types, such as neurons, astrocytes, Schwann cells, or oligodendrocytes, by treatment with a therapeutic hydrogenated pyrido[4,3-b]indole or a pharmaceutically acceptable salt thereof at a concentration ranging from about 1 pM to about 5 mM, from about 10 pM to about 500 μM, from about 50 pM to about 100 μM, from about 0.25 nM to about 20 μM, from about 1 nM to about 5 μM, from about 6 nM to about 800 nM, from about 30 nM to about 160 nM. In some embodiments, the therapeutic hydrogenated pyrido[4,3-b]indole or a pharmaceutically acceptable salt thereof is dimebon in saline. In certain embodiments, the MSCs differentiate into cortical neurons, hippocampal neurons, or spinal motor neurons. In certain embodiments, the MSCs are induced to differentiate into specific cell types, such as neurons, astrocytes, Schwann cells, or oligodendrocytes, by treatment with a therapeutic hydrogenated pyrido[4,3-b]indole or pharmaceutically acceptable salt thereof at a concentration of about 0.01 nM, 0.05 nM, 0.25 nM, 1.25 nM, 6.25 nM, 31.25 nM, 156.25 nM, 781 nM, 3.905 μM, 19.530 μM, 97.660 μM, or 488.280 μM. In some embodiments, the therapeutic hydrogenated pyrido[4,3-b]indole or a pharmaceutically acceptable salt thereof is dimebon in saline. In certain embodiments, the MSCs differentiate into cortical neurons, hippocampal neurons, or spinal motor neurons. In some embodiments, the MSCs are treated with a therapeutic hydrogenated pyrido[4,3-b]indole such as dimebon and a second compound, such as a growth factor, or an anti-cell death compound. If the MSCs are treated with such a combination of compounds, the compounds may be administered simultaneously or sequentially in any order.

In certain embodiments, the MSCs are neuronal-lineage-specific stem cells (i.e., neuronal stem cells) that have the potential to differentiate into at least two cell types selected from a neuron, an astrocyte, a Schwann cell, and an oligodendrocyte, and exhibit self-renewal. In certain embodiments, the MSCs are multipotential stem cells from other lineages. In certain embodiments, the neuronal stem cells differentiate into hippocampal neurons, cortical neurons, or spinal motor neurons. In certain embodiments, the non-neuronal stem cell differentiates into a skin cell, a cardiac muscle cell, a skeletal muscle cell, a liver cell, a kidney cell, or a cartilage cell. After the MSCs have been isolated, cultured, and differentiated by treatment with a therapeutic hydrogenated pyrido[4,3-b]indole or pharmaceutically acceptable salt thereof, such as dimebon in saline, cells of the desired type are then selected and purified from culture. Differentiated cells of the desired cell type can be purified from in vitro cell cultures, for example, by identifying cells positive for particular cell-type-specific surface markers (i.e., the neuron-specific marker NeuN and the like), and sorting cells positive or negative for the desired markers from a mixed population of cultured cells. Such sorting may be performed, for example, by flow cytometry or other established methods known to one of ordinary skill in the art.

In one aspect, the invention provides a method of differentiating multipotential stem cells comprising incubating cultured multipotential stem cells isolated from an individual with an amount of a hydrogenated pyrido[4,3-b]indole or pharmaceutically acceptable salt thereof effective to induce the multipotential stem cells to differentiate. In one embodiment, the hydrogenated pyrido[4,3-b]indole or pharmaceutically acceptable salt thereof is dimebon. In one embodiment, the multipotential stem cell is a neuronal stem cell or a non-neuronal stem cell. In one embodiment, the neuronal stem cell differentiates into a hippocampal neuron, a cortical neuron, or a spinal motor neuron. In one embodiment, the non-neuronal stem cell differentiates into a skin cell, a cardiac muscle cell, a skeletal muscle cell, a liver cell, a kidney cell, or a cartilage cell. In one embodiment, the method further comprises the step of incubating the multipotential stem cells with a growth factor and/or an anti-cell death compound. In one embodiment, the method further comprises the step of selecting a differentiated cell type from culture. In one embodiment, the selected differentiated cell type is a hippocampal neuron, a cortical neuron, or a spinal motor neuron. In one embodiment, the selected differentiated cell type is a skin cell, a cardiac muscle cell, a skeletal muscle cell, a liver cell, a kidney cell, or a cartilage cell.

### Therapeutic Methods Involving One or More Cells

Differentiated cells (i.e., neurons or non-neuronal cells) produced by the methods of the invention are useful for improving the treatment of a variety of neuronal and non-neuronal indications as described herein. Thus, in certain aspects, the invention features methods of improving the treatment of an individual suffering from any one of a variety of neuronal or non-neuronal indications by administering an effective amount of differentiated cells (i.e., neurons) produced by the methods of the invention. The effective amount of differentiated cells can be administered to an individual by any conventional method of administration known to one of ordinary skill in the art, including perfusion, injection, and surgical implantation. Administration can be systemic, for example, by intravenous administration, or local, for example by direct injection or surgical implantation at a particular site. Exemplary sites of administration include, for example, the site of an avulsion or spinal cord injury, in a particular region of the brain having lesions or other defects associated with neurodegeneration, or in a muscle group associated with symptoms of a neuronal indication, such as the facial muscles of an individual having myasthenia gravis. In some embodiments, the differentiated cells are from the same species as the individual being treated. In some embodiments, the differentiated cells are from the individual being treated or a relative of the individual being treated. In one embodiment, treatment of non-neuronal indications includes, but is not limited to, treatment of degenerative disorders or trauma, and the treatment includes administration of non-neuronal cells, such as cardiac cells for the treatment of heart disease, pancreatic islet cells for the treatment of diabetes, adipocytes for the treatment of anorexia or wasting associated with many diseases including AIDS, cancer, and cancer treatments, smooth muscle cells to be used in vascular grafts and intestinal grafts, cartilage to be used to treat cartilage injuries and degenerative conditions of cartilage and osteoarthritis, and replace cells damaged or lost to bacterial or viral infection, or those lost to traumatic injuries such as burns, fractures, and lacerations.

Cells that have been incubated with a hydrogenated pyrido[4,3-b]indole or a pharmaceutically acceptable salt thereof are useful to treat and/or prevent and/or delay the onset and/or the development of a condition for which the activation, differentiation, and/or proliferation of one or more cell types is beneficial in an individual, such as a human. In some embodiments, one or more cells (e.g., neuronal stem cells and/or neuronal cells or non-neuronal cells) are incubated with a therapeutic compound under conditions sufficient to activate the cell(s), promote the differentiation of the cell(s), promote the proliferation of the cell(s), or any combination of two or more of the foregoing. In some embodiments, the cell(s) are also incubated with a growth factor (e.g., a VEGF protein or a trophic growth factor) and/or an anti-cell death compound. In various embodiments, the cells(s) are incubated with a therapeutic compound before, during, or after they are incubated with a growth factor and/or an anti-cell death compound. An effective amount of the incubated cell(s) is administered to the individual. In some embodiments, a therapeutic compound, a growth factor, an anti-cell death compound, or any combination of two or more of the foregoing are also administered to the individual. The therapeutic compound, growth factor, and/or anti-cell death compound may be administered sequentially or simultaneously with the administration of the cell(s).

Accordingly, in one aspect, the invention provides a method of treating, preventing, delaying the onset, and/or delaying the development of a condition for which the activation, differentiation, and/or proliferation of one or more cell types is beneficial, the method comprising administering to an individual in need thereof an effective amount of a first therapy comprising a cell that has been incubated with a hydrogenated pyrido[4,3-b]indole or pharmaceutically acceptable salt thereof under conditions sufficient to activate the cell, promote the differentiation of the cell, promote the proliferation of the cell, or any combination of two or more of the foregoing. In one embodiment, the hydrogenated pyrido[4,3-b]indole or pharmaceutically acceptable salt thereof is dimebon. In one embodiment, the method further comprises administering a second therapy comprising a growth factor and/or anti-cell death compound to the individual. In one embodiment, the cell type is selected from the group consisting of multipotential stem cells, neuronal stem cells, non-neuronal cell and neurons. In one embodiment, the multipotential stem cell is a non-neuronal stem cell. In one embodiment, the cell type is a neuron, and the method increases the length of one or more axons of the neuron. In one embodiment, the cell type is a neuronal stem cell, and the method promotes the differentiation of the neuronal stem cell into a neuron. In one embodiment, the neuronal stem cell differentiates into a hippocampal neuron, cortical neuron, or spinal motor neuron. In one embodiment, the cell type is a non-neuronal stem cell, and the method promotes the differentiation of the non-neuronal stem cell into a skin cell, a cardiac muscle cell, a skeletal muscle cell, a liver cell, a kidney cell, or a cartilage cell.

Alternatively, cells that have not been previously incubated with a hydrogenated pyrido[4,3-b]indole or a pharmaceutically acceptable salt thereof can be administered to an individual (e.g., a human) to treat and/or prevent and/or delay the onset and/or the development of a condition for which the activation, differentiation, and/or proliferation of one or more cell types is beneficial. In some embodiments, a cell is administered in combination with a therapeutic compound to the individual. In some embodiments, a growth factor and/or an anti-cell death compound is also administered to the individual. In some embodiments, both a growth factor and an anti-cell death compound are administered to the individual. In various embodiments, the therapeutic compound, growth factor, and/or anti-cell death compound promotes the activation, differentiation, and/or proliferation of the administered cells in vivo. In some embodiments, the therapeutic compound, growth factor, and/or anti-cell death compound promotes the activation, differentiation, and/or proliferation of endogenous cells that were not transplanted into the individual. In some embodiments, the transplanted cell is from the same species as the individual being treated. In some embodiments, the transplanted cell is from the individual being treated or a relative of the individual being treated. The therapeutic compound, growth factor, and/or anti-cell death compound may be administered sequentially or simultaneously with the administration of the cell(s).

Accordingly, in one aspect, the invention provides a method of treating, preventing, delaying the onset, and/or delaying the development of a condition for which the activation, differentiation, and/or proliferation of one or more cell types is beneficial, the method comprising administering to an individual in need thereof an effective amount of a combination of (i) a first therapy comprising a cell and (ii) a second therapy comprising a hydrogenated pyrido[4,3-b]indole or pharmaceutically acceptable salt thereof. In one embodiment, the hydrogenated pyrido[4,3-b]indole or pharmaceutically acceptable salt thereof is dimebon. In one embodiment, the method further comprises administering a second therapy comprising a growth factor and/or anti-cell death compound to the individual. In one embodiment, the cell type is selected from the group consisting of multipotential stem cells, neuronal stem cells, non-neuronal cell and neurons. In one embodiment, the cell type is a neuron, and the method increases the length of one or more axons of the neuron. In one embodiment, the cell type is a neuronal stem cell, and the method promotes the differentiation of the neuronal stem cell into a neuron. In one embodiment, the neuronal stem cell differentiates into a hippocampal neuron, cortical neuron, or spinal motor neuron. In one embodiment, the multipotential stem cells are non-neuronal stem cells. In one embodiment, the non-neuronal stem cell differentiates into a skin cell, a cardiac muscle cell, a skeletal muscle cell, a liver cell, a kidney cell, or a cartilage cell. In one embodiment, the first and second therapies are administered sequentially. In one embodiment, the first and second therapies are administered simultaneously. In one embodiment, the first and second therapies are contained in the same pharmaceutical composition. In one embodiment, the first and second therapies are contained in separate pharmaceutical compositions. In one embodiment, the first and second therapies have at least an additive effect. In one embodiment, the first and second therapies have a synergistic effect.

In another aspect, the invention provides a method of aiding in the treatment of an individual, comprising administering to the individual a first therapy comprising a multipotential stem cell and a second therapy comprising an amount of a hydrogenated pyrido[4,3-b]indole or pharmaceutically acceptable salt thereof effective to induce the multipotential stem cell to differentiate. In one embodiment, the hydrogenated pyrido[4,3-b]indole or pharmaceutically acceptable salt thereof is dimebon. In one embodiment, the method further comprises administering a second therapy comprising a growth factor and/or anti-cell death compound to the individual. In one embodiment, the multipotential stem cell is a neuronal stem cell or a non-neuronal stem cell. In one embodiment, the neuronal stem cell differentiates into a hippocampal neuron, a cortical neuron, or a spinal neuron. In one embodiment, the non-neuronal stem cell differentiates into a skin cell, a cardiac muscle cell, a skeletal muscle cell, a liver cell, a kidney cell, or a cartilage cell. In one embodiment, the first and second therapies are administered sequentially. In one embodiment, the first and second therapies are administered simultaneously. In one embodiment, the first and second therapies are contained in the same pharmaceutical composition. In one embodiment, the first and second therapies are contained in separate pharmaceutical compositions. In one embodiment, the first and second therapies have at least an additive effect. In one embodiment, the first and second therapies have a synergistic effect.

In another aspect, the invention provides a method of aiding in the treatment of an individual having a neuronal indication or a non-neuronal indication comprising administering to the individual differentiated cells produced by any of the methods described herein. In one embodiment, the differentiated cells are hippocampal neurons, cortical neurons, or spinal motor neurons. In one embodiment, the differentiated cells are non-neuronal cells. In certain embodiments, the differentiated cells are skin cells, cardiac muscle cells, skeletal muscle cells, liver cells, or kidney cells. In one embodiment, the non-neuronal cells are skin cells. In embodiment, the differentiated cells are administered systemically by intravenous injection. In one embodiment, the differentiated cells are administered locally by direct injection or surgical implantation.

In some embodiments, a dose of a therapeutic compound is administered orally, intravenously, intraperitoneally, subcutaneously, intrathecally, intramuscularly, intraocularly, transdermally, or topically (i.e., as eye drops or ear drops). In some embodiments, a dose of a therapeutic compound is administered once daily, twice daily, three times daily, or at higher frequencies. In some embodiments, a dose of a therapeutic composition is administered once a week, twice a week, three times a week, four times a week, or at higher frequencies. In some embodiments, a dose of a therapeutic compound is administered as a controlled release formulation every week, two weeks, every three weeks, every four weeks, every five weeks, every six weeks, or at even longer intervals. In some embodiments, a dose (e.g., a dose for oral administration) of about 1 ng/day, 10 ng/day, 100 ng/day, 250 ng/day, 500 ng/day, 1 μg/day, 5 μg/day, 10 μg/day, 20 μg/day, 25 μg/day, 40 μg/day, 80 μg/day, 120 μg/day, 160 μg/day, 320 μg/day, or 120 mg/day of a therapeutic compound is administered. In some embodiments, the therapeutic compound is administered directly by infusion to the brain (e.g., intrathecal or intraventricular administration) at a dose of about 1 ng/day, 10 ng/day, 100 ng/day, 250 ng/day, 500 ng/day, 1 μg/day, 5 μg/day, 10 μg/day, 20 μg/day, 25 μg/day, 40 μg/day, 80 μg/day, 120 μg/day, 160 μg/day, 320 μg/day, or 120 mg/day. In some embodiments, a slow release pump or other device in the brain issued to administer any of the doses described herein. In some variations, the therapeutic hydrogenated pyrido[4,3-b]indole or pharmaceutically acceptable salt thereof is dimebon.

### Additional Methods of the Invention

In one aspect, the invention provides a method of treating, preventing, delaying the onset, and/or delaying the development of a condition for which the activation, differentiation, and/or proliferation of one or more cell types is beneficial, the method comprising administering to an individual in need thereof an effective amount of a first therapy comprising a hydrogenated pyrido[4,3-b]indole or pharmaceutically acceptable salt thereof. In one embodiment, the cell type is selected from the group consisting of stem cells, neuronal stem cells, non-neuronal cells and neurons. In one embodiment, the cell type is a neuronal stem cell or a neuronal cell, and wherein the first therapy increases the length of one or more axons of the cell. In one embodiment, the cell type is a neuronal stem cell, and wherein the first therapy promotes the differentiation of the neuronal stem cell into a neuronal cell. In one embodiment, the neuronal stem cell differentiates into a hippocampal neuron, cortical neuron, or spinal motor neuron.

In one aspect, the invention provides a method of treating, preventing, delaying the onset, and/or delaying the development of a condition for which the activation, differentiation, and/or proliferation of one or more cell types is beneficial, the method comprising administering to an individual in need thereof an effective amount of a combination of (i) a first therapy comprising a hydrogenated pyrido[4,3-b]indole or pharmaceutically acceptable salt of any of the foregoing and (ii) a second therapy comprising a growth factor and/or anti-cell death compound. In one embodiment, the cell type is selected from the group consisting of stem cells, neuronal stem cells, non-neuronal cell and neurons. In one embodiment, the cell type is a neuronal stem cell or a neuronal cell, and wherein the method increases the length of one or more axons of the cell. In one embodiment, the cell type is a neuronal stem cell, and wherein the method promotes the differentiation of the neuronal stem cell into a neuronal cell. In one embodiment, the neuronal stem cell differentiates into a hippocampal neuron, cortical neuron, or spinal motor neuron.

In one aspect, the invention provides a method of treating, preventing, delaying the onset, and/or delaying the development of a condition for which the activation, differentiation, and/or proliferation of one or more cell types is beneficial, the method comprising administering to an individual in need thereof an effective amount of a first therapy comprising a cell that has been incubated with a hydrogenated pyrido[4,3-b]indole or pharmaceutically acceptable salt thereof under conditions sufficient to activate the cell, promote the differentiation of the cell, promote the proliferation of the cell, or any combination of two or more of the foregoing. In one embodiment, the method further comprises administering a second therapy comprising a hydrogenated pyrido[4,3-b]indole or pharmaceutically acceptable salt thereof to the individual. In one embodiment, the method further comprises administering a second therapy comprising a growth factor and/or anti-cell death compound to the individual. In one embodiment, the method further comprises administering a second therapy comprising a hydrogenated pyrido[4,3-b]indole or pharmaceutically acceptable salt thereof and administering a third therapy comprising a growth factor and/or anti-cell death compound to the individual. In one embodiment, the cell type is selected from the group consisting of stem cells, neuronal stem cells, non-neuronal cell and neurons. In one embodiment, the cell type is a neuronal stem cell or a neuronal cell, and wherein the method increases the length of one or more axons of the cell. In one embodiment, the cell type is a neuronal stem cell, and wherein the method promotes the differentiation of the neuronal stem cell into a neuronal cell. In one embodiment, the neuronal stem cell differentiates into a hippocampal neuron, cortical neuron, or spinal motor neuron.

In one aspect, the invention provides a method of treating, preventing, delaying the onset, and/or delaying the development of a condition for which the activation, differentiation, and/or proliferation of one or more cell types is beneficial, the method comprising administering to an individual in need thereof an effective amount of a combination of (i) a first therapy comprising a cell and (ii) a second therapy comprising a hydrogenated pyrido[4,3-b]indole or pharmaceutically acceptable salt thereof. In one embodiment, the method further comprises administering a third therapy comprising a growth factor and/or anti-cell death compound to the individual. In one embodiment, the cell type is selected from the group consisting of stem cells, neuronal stem cells, non-neuronal cells and neurons. In one embodiment, the cell type is a neuronal stem cell or a neuronal cell, and wherein the method increases the length of one or more axons of the cell. In one embodiment, the cell type is a neuronal stem cell, and wherein the method promotes the differentiation of the neuronal stem cell into a neuronal cell. In one embodiment, the neuronal stem cell differentiates into a hippocampal neuron, cortical neuron, or spinal motor neuron. In one embodiment, the cell has not been incubated with a hydrogenated pyrido[4,3-b]indole or pharmaceutically acceptable salt thereof prior to administration to the individual.

In any of the above embodiments, the first and second therapies are administered sequentially. In any of the above embodiments, the first and second therapies are administered simultaneously. In any of the above embodiments, the first and second therapies are contained in the same pharmaceutical composition. In any of the above embodiments, the first and second therapies are contained in the separate pharmaceutical compositions. In any of the above embodiments, the first and second therapies have at least an additive effect. In any of the above embodiments, the first and second therapies have a synergistic effect.

In one aspect, the invention provides, a method of activating a cell comprising incubating a cell with a hydrogenated pyrido[4,3-b]indole or pharmaceutically acceptable salt thereof under conditions sufficient to activate the cell. In one embodiment, the method further comprises incubating the cell with a growth factor and/or anti-cell death compound. In one embodiment, the cell is selected from the group consisting of stem cells, neuronal stem cells, non-neuronal cell and neurons. In one embodiment, the cell is a neuronal stem cell or a neuronal cell, and wherein the incubation increases the length of one or more axons of the cell. In one embodiment, the incubation occurs ex vivo. In one embodiment, the incubation occurs in vivo.

In one aspect, the invention provides a method of promoting the differentiation and/or proliferation of a cell comprising incubating a cell with a hydrogenated pyrido[4,3-b]indole or pharmaceutically acceptable salt thereof under conditions sufficient to promoting the differentiation and/or proliferation of the cell. In one embodiment, the method further comprises incubating the cell with a growth factor and/or anti-cell death compound. In one embodiment, the cell is selected from the group consisting of stem cells, neuronal stem cells, non-neuronal cell and neurons. In one embodiment, the cell is a neuronal stem cell that differentiates into a neuronal cell. In one embodiment, the cell is a neuronal stem cell that differentiates into a hippocampal neuron, cortical neuron, or spinal motor neuron. In one embodiment, the incubation occurs ex vivo. In one embodiment, the incubation occurs in vivo. In one aspect, the invention provides a purified cell made by any of the methods provided herein.

In any of the above embodiments, the hydrogenated pyrido[4,3-b]indole is a tetrahydro pyrido[4,3-b]indole. In any of the above embodiments, the hydrogenated pyrido[4,3-b]indole is a hexahydro pyrido[4,3-b]indole. In any of the above embodiments, the hydrogenated pyrido[4,3-b]indole is of the formula:

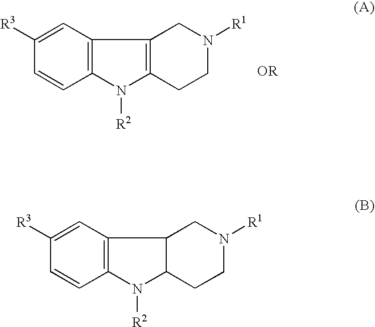

wherein R¹ is selected from a lower alkyl or aralkyl; R² is selected from a hydrogen, aralkyl or substituted heteroaralkyl; and R³ is selected from hydrogen, lower alkyl or halo. In any of the above embodiments, aralkyl is PhCH₂— and substituted heteroaralkyl is 6-CH₃-3-Py-(CH₂)₂—. In any of the above embodiments, R¹ is selected from CH₃—, CH₃CH₂—, or PhCH₂—; R² is selected from H—, PhCH₂—, or 6-CH₃-3-Py-(CH₂)₂—; and R³ is selected from H—, CH₃— or Br—. In any of the above embodiments, the hydrogenated pyrido[4,3-b]indole is selected from the group consisting of cis(±) 2,8-dimethyl-2,3,4,4a,5,9b-hexahydro-1H-pyrido[4,3-b]indole; 2-ethyl-2,3,4,5-tetrahydro-1H-pyrido[4,3-b]indole; 2-benzyl-2,3,4,5-tetrahydro-1H-pyrido[4,3-b]indole; 2,8-dimethyl-5-benzyl-2,3,4,5-tetrahydro-1H-pyrido[4,3-b]indole; 2-methyl-5-(2-methyl-3-pyridyl)ethyl-2,3,4,5-tetrahydro-1H-pyrido[4,3-b]indole; 2,8-dimethyl-5-(2-(6-methyl-3-pyridyl)ethyl)-2,3,4,5-tetrahydro-1H-pyrido[4,3-b]indole; 2-methyl-2,3,4,5-tetrahydro-1H-pyrido[4,3-b]indole; 2,8-dimethyl-2,3,4,5-tetrahydro-1H-pyrido[4,3-b]indole; 2-methyl-8-bromo-2,3,4,5-tetrahydro-1H-pyrido[4,3-b]indole. In any of the above embodiments, the hydrogenated pyrido[4,3-b]indole is 2,8-dimethyl-5-(2-(6-methyl-3-pyridyl)ethyl)-2,3,4,5-tetrahydro-1H-pyrido[4,3-b]indole. In any of the above embodiments, the pharmaceutically acceptable salt is a pharmaceutically acceptable acid salt. In any of the above embodiments, the pharmaceutically acceptable salt is a hydrochloride acid salt. In any of the above embodiments, the hydrogenated pyrido[4,3-b]indole is 2,8-dimethyl-5-(2-(6-methyl-3-pyridyl)ethyl)-2,3,4,5-tetrahydro-1H-pyrido[4,3-b]indole dihydrochloride.

In any of the above embodiments, R¹ is CH₃—, R² is H and R³ is CH₃—. In any of the above embodiments, R¹ is CH₃CH₂— or PhCH₂—, R² is H—, and R³ is CH₃—. In any of the above embodiments, R¹ is CH₃—, R² is PhCH₂—, and R³ is CH₃—. In any of the above embodiments, R¹ is CH₃—, R² is 6-CH₃-3-Py-(CH₂)₂—, and R³ is H—. In any of the above embodiments, R² is 6-CH₃-3-Py-(CH₂)₂—. In any of the above embodiments, R¹ is CH₃—, R² is H—, and R³ is H— or CH₃—. In any of the above embodiments, R¹ is CH₃—, R² is H—, and R³ is Br—. In any of the above embodiments, the growth factor comprises VEGF, IGF-1, FGF, NGF, BDNF, GCS-F, GMCS-F, or any combination of two or more of the foregoing.

In any of the above aspects or embodiments, the disease or indication is a neuronal or non-neuronal indication, such as Alzheimer's disease, impaired cognition associated with aging, age-associated hair loss, age-associated weight loss, age-associated vision disturbance, Huntington's disease, schizophrenia, canine cognitive dysfunction syndrome (CCDS), amyotrophic lateral sclerosis (ALS), Parkinson's disease, Lewy body disease, Menkes disease, Wilson disease, Creutzfeldt-Jakob disease, Fahr disease, an acute or chronic disorder involving cerebral circulation, such as stroke or cerebral hemorrhagic insult, age-associated memory impairment (AAMI), mild cognitive impairment (MCI), injury-related mild cognitive impairment (MCI), injury-related mild cognitive impairment (MCI) resulting from battlefield injuries, post-concussion syndrome, and adjuvant chemotherapy, neuronal death mediated ocular disease, macular degeneration, age-related macular degeneration, autism, including autism spectrum disorder, Asperger syndrome, and Rett syndrome, an avulsion injury, a spinal cord injury, myasthenia gravis, Guillain-Barré syndrome, multiple sclerosis, diabetic neuropathy, fibromyalgia, neuropathy associated with spinal cord injury, heart disease, diabetes, anorexia, AIDS- or chemotherapy-associated wasting, vascular injury, intestinal injury, cartilage injury, osteoarthritis, bacterial infection, viral infection, a first-, second-, or third-degree burn, a simple, compound, stress, or compression fracture, or a laceration.

In any of the above aspects or embodiments, the disease or condition is a neuronal indication such as Alzheimer's disease, impaired cognition associated with aging, age-associated hair loss, age-associated weight loss, age-associated vision disturbance, Huntington's disease, schizophrenia, canine cognitive dysfunction syndrome (CCDS), amyotrophic lateral sclerosis (ALS), Parkinson's disease, Lewy body disease, Menkes disease, Wilson disease, Creutzfeldt-Jakob disease, Fahr disease, an acute or chronic disorder involving cerebral circulation, such as stroke or cerebral hemorrhagic insult, age-associated memory impairment (AAMI), mild cognitive impairment (MCI),injury-related mild cognitive impairment (MCI), injury-related mild cognitive impairment (MCI) resulting from battlefield injuries, post-concussion syndrome, and adjuvant chemotherapy, neuronal death mediated ocular disorder, macular degeneration, age-related macular degeneration, autism, including autism spectrum disorder, Asperger syndrome, and Rett syndrome, an avulsion injury, a spinal cord injury, myasthenia gravis, Guillain-Barré syndrome, multiple sclerosis, diabetic neuropathy, fibromyalgia, or neuropathy associated with spinal cord injury.

In any of the above aspect or embodiments, the disease or condition is a neuronal indication, such as Alzheimer's disease, impaired cognition associated with aging, age-associated hair loss, age-associated weight loss, age-associated vision disturbance, Huntington's disease, schizophrenia, canine cognitive dysfunction syndrome (CCDS), amyotrophic lateral sclerosis (ALS), Parkinson's disease, Lewy body disease, Menkes disease, Wilson disease, Creutzfeldt-Jakob disease, Fahr disease, an acute or chronic disorder involving cerebral circulation, such as stroke or cerebral hemorrhagic insult, age-associated memory impairment (AAMI), or mild cognitive impairment (MCI). In any of the above aspects or embodiments, the disease or condition is not Alzheimer's disease. In any of the above aspects or embodiments, the disease or condition is not amyotrophic lateral sclerosis (ALS). In any of the above aspects or embodiments, the disease or condition is neither Alzheimer's disease nor amyotrophic lateral sclerosis (ALS). In any of the above aspects or embodiments, the disease or condition is not Huntington's disease. In any of the above aspects or embodiments, the disease or condition is not schizophrenia. In any of the above aspects or embodiments, the disease or condition is not MCI. In one variation, the individual is a human who has not been diagnosed with and/or is not considered at risk for developing Alzheimer's disease, Huntington's disease, amyotrophic lateral sclerosis, or schizophrenia. In one variation, the individual is a human who does not have impaired cognition associated with aging or does not have a non-life threatening condition associated with the aging process (such as loss of sight (cataract), deterioration of the dermatohairy integument (alopecia) or an age-associated decrease in weight due to the death of muscular and fatty cells) or a combination thereof. In any of the above aspects or embodiments, the disease or condition is a neuronal indication, such as injury-related mild cognitive impairment (MCI), injury-related mild cognitive impairment (MCI) resulting from battlefield injuries, post-concussion syndrome, and adjuvant chemotherapy, neuronal death mediated ocular disorder, macular degeneration, age-related macular degeneration, autism, including autism spectrum disorder, Asperger syndrome, and Rett syndrome, an avulsion injury, a spinal cord injury, myasthenia gravis, Guillain-Barré syndrome, multiple sclerosis, diabetic neuropathy, fibromyalgia, or neuropathy associated with spinal cord injury. In any of the above aspects or embodiments, the disease or condition is a non-neuronal indication, such as heart disease, diabetes, anorexia, AIDS- or chemotherapy-associated wasting, vascular injury, intestinal injury, cartilage injury, osteoarthritis, bacterial infection, viral infection, a first-, second-, or third-degree burn, a simple, compound, stress, or compression fracture, or a laceration.

### Exemplary Conditions

Any disease or condition for which the activation, differentiation, and/or proliferation of one or more cell types is beneficial can be prevented, treated, inhibited, and/or delayed using the methods of the invention. Also within the invention is a method of inhibiting cell death (e.g., neuronal cell death or non-neuronal cell death) associated with a disease or condition described herein. In another embodiment, the present invention provides a method of preventing or slowing the onset and/or development of a disease or condition in an individual who has a mutated or abnormal gene associated with the disease or condition (e.g., an APP mutation, a presenilin mutation and/or an ApoE4 allele associated with Alzheimer's disease if the disease or condition to be treated is Alzheimer's disease). In another embodiment, the present invention provides a method of slowing the progression of a disease or condition in an individual who has been diagnosed with a disease or condition. In another embodiment, the present invention provides a method of preventing or slowing the onset and/or development of a disease or condition in an individual who is at risk of developing a disease or condition (e.g., an individual with an APP mutation, a presenilin mutation and/or an ApoE4 allele associated with Alzheimer's disease if the disease or condition to be treated is Alzheimer's disease).

Any of the methods described herein for treating, preventing, delaying the onset and/or development of or otherwise concerning administration of compounds of the invention to an individual in connection with a disease or condition may involve administering to an individual the compounds of the invention as a monotherapy (such as administering a therapeutic compound or a pharmaceutically acceptable salt thereof) or as a combination therapy (such as administering a therapeutic compound and a growth factor and/or anti-cell death compound and/or a cell that has been incubated as described herein). In various embodiments, the method comprises administering to an individual an effective amount of any of the following: (1) a therapeutic compound or pharmaceutically acceptable salt thereof, (2) a combination of (i) a therapeutic compound or pharmaceutically acceptable salt thereof and (ii) a growth factor and/or an anti-cell death compound, (3) a cell that has been incubated with a therapeutic compound or pharmaceutically acceptable salt thereof (4) a combination of (i) a therapeutic compound or pharmaceutically acceptable salt thereof and (ii) a cell that has been incubated with a therapeutic compound or pharmaceutically acceptable salt thereof, (5) a combination of (i) a therapeutic compound or pharmaceutically acceptable salt thereof, (ii) a cell that has been incubated with a therapeutic compound or pharmaceutically acceptable salt thereof, and (iii) a growth factor and/or an anti-cell death compound, (6) a combination of (i) a therapeutic compound or pharmaceutically acceptable salt thereof and (ii) a cell (such as a cell that has not been incubated with a therapeutic compound or pharmaceutically acceptable salt thereof), or (7) a combination of (i) a therapeutic compound or pharmaceutically acceptable salt thereof, (ii) a cell (such as a cell that has not been incubated with a therapeutic compound or pharmaceutically acceptable salt thereof), and (iii) a growth factor and/or an anti-cell death compound.

Exemplary conditions that can be prevented, treated, inhibited, and/or delayed using the methods of the invention include: Alzheimer's disease; Huntington's disease; neuronal death mediated ocular disease, including neuronal death mediated ocular diseases that involve death of photoreceptor cells or involve retinal cell death or involve neuron death by apoptosis (macular degeneration (dry form macular degeneration or Stargardt Macular Degeneration (STGD)), glaucoma, retinitis pigmentosa, congenital stationary night blindness (Oguchi disease), childhood onset severe retinal dystrophy, Leber congenital amaurosis, Bardet-Biedle syndrome, Usher syndrome, blindness from an optic neuropathy, Leber's hereditary optic neuropathy, color blindness and Hansen-Larson-Berg syndrome); amyotrophic lateral sclerosis (ALS); Parkinson's disease; Lewy body disease; Menkes disease; Wilson disease; Creutzfeldt-Jakob disease; Fahr disease; schizophrenia; Canine Cognitive Dysfunction Syndrome; an acute or chronic disorder involving cerebral circulation, such as stroke, or cerebral hemorrhagic insult (examples of indications for which the method of the invention may be used include, but are not limited to, stroke, reduction of cerebral blood flow (ischemia), and other events involving impaired cerebral circulation or cerebral hemorrhagic insult, such as may occur upon trauma, including trauma to the head), method of lessening the severity of disability due to neurological deficit (e.g., paresis or paralysis) that is associated with an acute or chronic insufficiency of cerebral circulation and/or ischemic or hemorrhagic insult in an individual in need thereof. Also embraced is a method of enhancing the cognitive functions of an individual who has suffered from neuronal cell death due to an acute insufficiency of cerebral circulation and/or ischemic or hemorrhagic insult. In one variation, the method comprises restoring or preventing a worsening of arterial patency (tissue activator) and/or preventing the development or worsening of thrombogenesis (fibrinolytics, anticoagulants, antiaggregants) and/or preventing or slowing the onset and/or progression of the death of viable neurons in an individual who is experiencing or has had an acute insufficiency of cerebral circulation and/or ischemic or hemorrhagic insult; Age-Associated Memory Impairment (AAMI) mild cognitive impairment (MCI), injury-related mild cognitive impairment (MCI), injury-related mild cognitive impairment (MCI) resulting from battlefield injuries, post-concussion syndrome, and adjuvant chemotherapy; slowing aging in an individual, for example by delaying the onset and/or slowing the progression of an aging-associated or age-related manifestation and/or pathology or condition, including, but not limited to, disturbance in skin-hair integument (such as baldness or alopecia), vision disturbance (such as development of cataracts), and weight loss (including weight loss due to the death of muscular and/or fatty cells). Exemplary diseases or conditions also include other neuronal indications, such as autism, including autism spectrum disorder, Asperger syndrome, and Rett syndrome, nerve damage resulting from avulsion injury or spinal cord injury, myasthenia gravis, Guillain-Barré syndrome, multiple sclerosis, diabetic neuropathy, fibromyalgia, neuropathy associated with herpes zoster infection, neuropathy associated with spinal cord injury. Exemplary diseases or conditions also include numerous non-neuronal indications, such as heart disease, diabetes, anorexia, AIDS- or chemotherapy-associated wasting, vascular injury, intestinal injury, cartilage injury, osteoarthritis, bacterial infection, viral infection, a first-, second-, or third-degree burn, a simple, compound, stress, or compression fracture, or a laceration. Exemplary conditions further include any of the diseases or conditions described in: U.S. Pat. No. 7,071,206 (“Agents for Treating Neurodegenerative Disorders,” U.S. application Ser. No. 11/004,001, filed Dec. 2, 2004); U.S. application Ser. No. 11/644,698 (“Methods and Compositions for Slowing Aging,” filed Dec. 22, 2006); U.S. patent application Ser. Nos. 11/543,529 and 11/543,341 (“Methods and Compositions for Treating Huntington's Disease,” filed Oct. 4, 2006); U.S. patent application Ser. No. 11/698,318 (“Methods and Compositions for Treating Schizophrenia,” filed Jan. 25, 2007); PCT Application No. PCT/U.S.07/20483 (filed Sep. 20, 2007) (“Hydrogenated pyrido[4,3-b]indoles such as Dimebon for Treating Canine Cognitive Dysfunction Syndrome”); U.S. Provisional Patent Application No. 60/846,184 (filed Sep. 20, 2006), PCT Application No. PCT/U.S.07/20516 (filed Sep. 20, 2007) (“Methods and Compositions for Treating Amyotrophic Lateral Sclerosis”); and PCT Application No. PCT/U.S.07/22645 (filed Oct. 26, 2008) (“Methods and Combination Therapies for Treating Alzheimer's Disease”), which are hereby incorporated by reference in their entireties, particularly with respect to diseases and conditions.

### Methods for Use in Neuronal Indications

In some embodiments, the methods of the invention are used to treat, prevent, delay the onset, and/or delay the development of a neuronal condition. For the treatment of neurological conditions such as neurodegenerative disorders, compositions that inhibit neuronal death, maintain neuronal phenotype, repair neuronal damage, promote the proliferation of neurons, promote the differentiation of neurons, promote the activation of neurons (such as neurite outgrowth) or any combination of two or more of the foregoing are desirable. Injury-induced expression of neurotrophic factors and corresponding receptors may play an important role in the ability of nerve regeneration. Neurotrophins like GDNF (Hiwasa et al., Neurosci. Letts. 238:115-118, 1997; Nakajima et al., Brain Res. 916:76-84, 2001), BDNF, and NGF (Wozniak, 1993) have been shown to maintain the survival and function of dopaminergic neurons in vitro and increase neurite outgrowth measured as the number and length of neurites (Hiwasa et al., Neurosci. Letts. 238:115-118, 1997; Nakajima et al., Brain Res. 916:76-84, 2001; Wozniak, Folia Morphol (Warsz). 1993; 52(4):173-81). Neurite outgrowth is a process by which neurons achieve connectivity and is stimulated by neuronal growth factors, neurotransmitters, and electrical activity. This process involves ligand-dependent activation of G-protein coupled receptors, such as D2 dopamine and cortical neurons, serotonin-1B receptors and thalamic neurons, CB1 cannabinoid receptor in Neuro2A cells, cilliary neurotrophic factor (CNTF), neurotrophin-3, and FGF (acidic/basic) in a variety of neurons.

Additionally, several findings indicate that neurogenesis is the natural repair pathway in the brain (Crespel et al., Rev. Neurol. (Paris). 2004, 160(12):1150-8). Hippocampal neurogenesis seems to contribute directly to cognitive capacity, which is supported by the finding that inhibiting neurogenesis causes memory impairment (Shors et al., Nature, 2001, 410(6826):372-6. Erratum in: Nature 2001 414(6866):938). Additionally, cognitive training increases formation of new neurons in this area (Gomez-Pinilla et al., Brain Res. 2001 Jun. 15; 904(1):13-9). This phenomenon can be also induced by physical exercise (Van Praag et al., Proc. Nat'l Acad. Sci. USA 1999, 96(23):13427-31), application of growth factors, or other compounds such as Lithium, Valproate or antidepressants (Bauer et al., 2003).

Various methods are disclosed herein, such as a method of extending neuronal survival and/or enhancing neuronal function and/or inhibiting cell death, which may include decreasing the amount of and/or extent of neuronal death or delaying the onset of neuronal death. The methods described may also be used in a method of treating and/or preventing and/or delaying the onset and/or development of an indication that is associated with neuronal cell death, such as a neurodegenerative disease or other indication or condition, including but not limited to the indications and conditions described in more detail herein. For any method described herein, including all methods described for particular indications, in one variation the method comprises administering to an individual an effective amount of any of the following: (1) a therapeutic compound or pharmaceutically acceptable salt thereof, (2) a combination of (i) a therapeutic compound or pharmaceutically acceptable salt thereof and (ii) a growth factor and/or an anti-cell death compound, (3) a cell that has been incubated with a therapeutic compound or pharmaceutically acceptable salt thereof (4) a combination of (i) a therapeutic compound or pharmaceutically acceptable salt thereof and (ii) a cell that has been incubated with a therapeutic compound or pharmaceutically acceptable salt thereof, (5) a combination of (i) a therapeutic compound or pharmaceutically acceptable salt thereof, (ii) a cell that has been incubated with a therapeutic compound or pharmaceutically acceptable salt thereof, and (iii) a growth factor and/or an anti-cell death compound, (6) a combination of (i) a therapeutic compound or pharmaceutically acceptable salt thereof and (ii) a cell (such as a cell that has not been incubated with a therapeutic compound or pharmaceutically acceptable salt thereof), or (7) a combination of (i) a therapeutic compound or pharmaceutically acceptable salt thereof, (ii) a cell (such as a cell that has not been incubated with a therapeutic compound or pharmaceutically acceptable salt thereof), and (iii) a growth factor and/or an anti-cell death compound.

In one aspect, the invention provides methods of treating, preventing, delaying the onset, and/or delaying the development of a condition, the method comprising administering to an individual in need thereof an effective amount of a first therapy comprising a hydrogenated pyrido[4,3-b]indole or pharmaceutically acceptable salt thereof, wherein the individual has injury-related mild cognitive impairment (MCI), neuronal death mediated ocular disease, macular degeneration, autism, autism spectrum disorder, Asperger syndrome, Rett syndrome, an avulsion injury, a spinal cord injury, myasthenia gravis, Guillain-Barré syndrome, multiple sclerosis, neuropathy, and non-neuronal indications. In certain embodiments, the individual has injury-related MCI resulting from battlefield injuries, post-concussion syndrome, or adjuvant chemotherapy. In one embodiment, the individual has a disorder for which the activation, differentiation, and/or proliferation of one or more cell types is beneficial for treating, preventing, delaying the onset, and/or delaying the development of the condition. In one embodiment, the invention provides a method of treating a condition for which the activation, differentiation, and/or proliferation of one or more cell types is beneficial. In one embodiment, the invention provides a method of preventing a condition for which the activation, differentiation, and/or proliferation of one or more cell types is beneficial. In one embodiment, the invention provides a method of delaying the onset of a condition for which the activation, differentiation, and/or proliferation of one or more cell types is beneficial. In one embodiment, the invention provides a method of delaying the development of a condition for which the activation, differentiation, and/or proliferation of one or more cell types is beneficial. In certain embodiments, the hydrogenated pyrido[4,3-b]indole is dimebon. In certain embodiments, the neuropathy is diabetic neuropathy, fibromyalgia, and neuropathy associated with spinal cord injury. In one embodiment, the neuropathy is diabetic neuropathy. In one embodiment, the neuropathy is fibromyalgia. In one embodiment, the neuropathy is neuropathy associated with spinal cord injury. In certain embodiments, the non-neuronal indication is heart disease, diabetes, anorexia, AIDS- or chemotherapy-associated wasting, vascular injury, intestinal injury, cartilage injury, osteoarthritis, bacterial infection, viral infection, a first-, second-, or third-degree burn, a simple, compound, stress, or compression fracture of a bone, or a laceration.

In one aspect, the invention provides a method of treating, preventing, delaying the onset, and/or delaying the development of a condition for which the activation, differentiation, and/or proliferation of one or more cell types is beneficial, the method comprising administering to an individual in need thereof an effective amount of a combination of (i) a first therapy comprising a hydrogenated pyrido[4,3-b]indole or pharmaceutically acceptable salt of any of the foregoing and (ii) a second therapy comprising a growth factor and/or anti-cell death compound. In one embodiment, the hydrogenated pyrido[4,3-b]indole is dimebon. In one embodiment, the cell type is selected from the group consisting of multipotential stem cells, neuronal stem cells, non-neuronal cell and neurons. In one embodiment, the cell type is a neuron, and the method increases the length of one or more axons of the neuron. In one embodiment, the cell type is a neuronal stem cell, and the method promotes the differentiation of the neuronal stem cell into a neuron. In one embodiment, the neuronal stem cell differentiates into a hippocampal neuron, cortical neuron, or spinal motor neuron. In one embodiment, the multipotential stem cell is a non-neuronal stem cell and the method promotes the differentiation of the non-neuronal stem cell. In certain embodiments, the non-neuronal stem cell differentiates into a skin cell, a cardiac muscle cell, a skeletal muscle cell, a liver cell, a kidney cell, or a cartilage cell. In one embodiment, the first and second therapies are administered sequentially. In one embodiment, the first and second therapies are administered simultaneously. In one embodiment, the first and second therapies are contained in the same pharmaceutical composition. In one embodiment, the first and second therapies are contained in separate pharmaceutical compositions. In one embodiment, the first and second therapies have at least an additive effect. In one embodiment, the first and second therapies have a synergistic effect.

In one aspect, the invention provides methods of stimulating neurite outgrowth in an individual comprising treating the individual with an amount of a therapeutic hydrogenated pyrido[4,3-b]indole or pharmaceutically acceptable salt thereof effective to stimulate neurite outgrowth. In certain embodiments, the individual has injury-related mild cognitive impairment (MCI), neuronal death mediated ocular disease, macular degeneration, autism, autism spectrum disorder, Asperger syndrome, Rett syndrome, an avulsion injury, a spinal cord injury, myasthenia gravis, Guillain-Barré syndrome, multiple sclerosis, neuropathy, and non-neuronal indications. In certain embodiments, the individual has injury-related MCI resulting from battlefield injuries, post-concussion syndrome, or adjuvant chemotherapy. In certain embodiments, the neuropathy is diabetic neuropathy, fibromyalgia, and neuropathy associated with spinal cord injury. In one embodiment, the neuropathy is diabetic neuropathy. In one embodiment, the neuropathy is fibromyalgia. In one embodiment, the neuropathy is neuropathy associated with spinal cord injury. In certain embodiments, the non-neuronal indications include heart disease, diabetes, anorexia, AIDS- or chemotherapy-associated wasting, vascular injury, intestinal injury, cartilage injury, osteoarthritis, bacterial infection, viral infection, a first-, second-, or third-degree burn, a simple, compound, stress, or compression fracture of a bone, or a laceration. In certain embodiments, the therapeutic hydrogenated pyrido[4,3-b]indole or a pharmaceutically acceptable salt thereof is dimebon. In one variation, the method further comprises administration of a growth factor and/or an anti-cell death compound. In some embodiments, a dose of a therapeutic compound is administered orally, intravenously, intraperitoneally, subcutaneously, intrathecally, intramuscularly, intraocularly, transdermally, or topically (i.e., as eye drops or ear drops). In some embodiments, a dose of a therapeutic compound is administered once daily, twice daily, three times daily, or at higher frequencies. In some embodiments, a dose of a therapeutic composition is administered once a week, twice a week, three times a week, four times a week, or at higher frequencies. In some embodiments, a dose of a therapeutic compound is administered as a controlled release formulation every week, every two weeks, every three weeks, every four weeks, every five weeks, every six weeks, or at even longer intervals. In some embodiments, a dose (e.g., a dose for oral administration) of about 1 ng/day, 10 ng/day, 100 ng/day, 250 ng/day, 500 ng/day, 1 μg/day, 5 μg/day, 10 μg/day, 20 μg/day, 25 μg/day, 40 μg/day, 80 μg/day, 125 μg/day, 160 μg/day, 320 μg/day, or 120 mg/day of a therapeutic hydrogenated pyrido[4,3-b]indole or pharmaceutically acceptable salt thereof is administered. In some embodiments, the therapeutic hydrogenated pyrido[4,3-b]indole or pharmaceutically acceptable salt thereof is administered directly by infusion to the brain (e.g., intrathecal or intraventricular administration) at a dose of about 1 ng/day, 10 ng/day, 100 ng/day, 250 ng/day, 500 ng/day, 1 μg/day, 5 mg/day, 10 μg/day, 20 μg/day, 25 μg/day, 40 μg/day, 80 μg/day, 125 μg/day, 160 μg/day, 320 μg/day, or 120 mg/day. In some embodiments, a slow release pump or other device in the brain issued to administer any of the doses described herein. In some variations, the therapeutic hydrogenated pyrido[4,3-b]indole or pharmaceutically acceptable salt thereof is dimebon.

In another aspect, the invention provides methods of enhancing neurogenesis in an individual comprising treating the individual with an amount of a therapeutic hydrogenated pyrido[4,3-b]indole or a pharmaceutically acceptable salt thereof effective to enhance neurogenesis. In certain embodiments, the individual has injury-related mild cognitive impairment (MCI), neuronal death mediated ocular disease, macular degeneration, autism, autism spectrum disorder, Asperger syndrome, Rett syndrome, an avulsion injury, a spinal cord injury, myasthenia gravis, Guillain-Barré syndrome, multiple sclerosis, neuropathy, and non-neuronal indications. In certain embodiments, the individual has injury-related MCI resulting from battlefield injuries, post-concussion syndrome, or adjuvant chemotherapy. In certain embodiments, the neuropathy is diabetic neuropathy, fibromyalgia, and neuropathy associated with spinal cord injury. In one embodiment, the neuropathy is diabetic neuropathy. In one embodiment, the neuropathy is fibromyalgia. In one embodiment, the neuropathy is neuropathy associated with spinal cord injury. In certain embodiments, the non-neuronal indications include heart disease, diabetes, anorexia, AIDS- or chemotherapy-associated wasting, vascular injury, intestinal injury, cartilage injury, osteoarthritis, bacterial infection, viral infection, a first-, second-, or third-degree burn, a simple, compound, stress, or compression fracture of a bone, or a laceration. In certain embodiments, the therapeutic hydrogenated pyrido[4,3-b]indole or pharmaceutically acceptable salt thereof is dimebon. In one variation, the method further comprises administration of a growth factor and/or an anti-cell death compound. In some embodiments, a dose of a therapeutic compound is administered orally, intravenously, intraperitoneally, subcutaneously, intrathecally, intramuscularly, intraocularly, transdermally, or topically (i.e., as eye drops or ear drops). In some embodiments, a dose of a therapeutic compound is administered once daily, twice daily, three times daily, or at higher frequencies. In some embodiments, a dose of a therapeutic composition is administered once a week, twice a week, three times a week, four times a week, or at higher frequencies. In some embodiments, a dose of a therapeutic compound is administered as a controlled release formulation every week, every two weeks, every three weeks, every four weeks, every five weeks, every six weeks, or at even longer intervals. In some embodiments, a dose (e.g., a dose for oral administration) of about 1 ng/day, 10 ng/day, 100 ng/day, 250 ng/day, 500 ng/day, 1 μg/day, 5 μg/day, 10 μg/day, 20 μg/day, 25 μg/day, 40 μg/day, 80 μg/day, 125 μg/day, 160 μg/day, 320 μg/day, or 120 mg/day of a therapeutic hydrogenated pyrido[4,3-b]indole or pharmaceutically acceptable salt thereof is administered. In some embodiments, the therapeutic hydrogenated pyrido[4,3-b]indole or pharmaceutically acceptable salt thereof is administered directly by infusion to the brain (e.g., intrathecal or intraventricular administration) at a dose of about 1 ng/day, 10 ng/day, 100 ng/day, 250 ng/day, 500 ng/day, 1 μg/day, 5 μg/day, 10 μg/day, 20 μg/day, 40 μg/day, 80 μg/day, 160 μg/day, 320 μg/day, or 120 mg/day. In some embodiments, a slow release pump or other device in the brain issued to administer any of the doses described herein. In some variations, the therapeutic hydrogenated pyrido[4,3-b]indole or pharmaceutically acceptable salt thereof is dimebon.

In still another aspect, the invention provides methods of stimulating neurite outgrowth and enhancing neurogenesis in an individual comprising treating the individual with an amount of a therapeutic hydrogenated pyrido[4,3-b]indole or a pharmaceutically acceptable salt thereof effective to stimulate neurite outgrowth and to enhance neurogenesis. In certain embodiments, the individual has injury-related mild cognitive impairment (MCI), neuronal death mediated ocular disease, macular degeneration, autism, autism spectrum disorder, Asperger syndrome, Rett syndrome, an avulsion injury, a spinal cord injury, myasthenia gravis, Guillain-Barré syndrome, multiple sclerosis, neuropathy, and non-neuronal indications. In certain embodiments, the individual has injury-related MCI resulting from battlefield injuries, post-concussion syndrome, or adjuvant chemotherapy. In certain embodiments, the neuropathy is diabetic neuropathy, fibromyalgia, and neuropathy associated with spinal cord injury. In one embodiment, the neuropathy is diabetic neuropathy. In one embodiment, the neuropathy is fibromyalgia. In one embodiment, the neuropathy is neuropathy associated with spinal cord injury. In certain embodiments, the non-neuronal indications include heart disease, diabetes, anorexia, AIDS- or chemotherapy-associated wasting, vascular injury, intestinal injury, cartilage injury, osteoarthritis, bacterial infection, viral infection, a first-, second-, or third-degree burn, a simple, compound, stress, or compression fracture of a bone, or a laceration. In certain embodiments, the therapeutic hydrogenated pyrido[4,3-b]indole or pharmaceutically acceptable salt thereof is dimebon. In one variation, the method further comprises administration of a growth factor and/or an anti-cell death compound. In some embodiments, a dose of a therapeutic compound is administered orally, intravenously, intraperitoneally, subcutaneously, intrathecally, intramuscularly, intraocularly, transdermally, or topically (i.e., as eye drops or ear drops). In some embodiments, a dose of a therapeutic compound is administered once daily, twice daily, three times daily, or at higher frequencies. In some embodiments, a dose of a therapeutic composition is administered once a week, twice a week, three times a week, four times a week, or at higher frequencies. In some embodiments, a dose of a therapeutic compound is administered as a controlled release formulation every week, every two weeks, every three weeks, every four weeks, every five weeks, every six weeks, or at even longer intervals. In some embodiments, a dose (e.g., a dose for oral administration) of about 1 ng/day, 10 ng/day, 100 ng/day, 250 ng/day, 500 ng/day, 1 μg/day, 5 μg/day, 10 μg/day, 20 μg/day, 25 μg/day, 40 μg/day, 80 μg/day, 125 μg/day, 160 μg/day, 320 μg/day, or 120 mg/day of a therapeutic hydrogenated pyrido[4,3-b]indole or pharmaceutically acceptable salt thereof is administered. In some embodiments, the therapeutic hydrogenated pyrido[4,3-b]indole or pharmaceutically acceptable salt thereof is administered directly by infusion to the brain (e.g., intrathecal or intraventricular administration) at a dose of about 1 ng/day, 10 ng/day, 100 ng/day, 250 ng/day, 500 ng/day, 1 μg/day, 5 μg/day, 10 μg/day, 20 μg/day, 25 μg/day, 40 μg/day, 80 μg/day, 125 μg/day, 160 μg/day, 320 μg/day, or 120 mg/day. In some embodiments, a slow release pump or other device in the brain issued to administer any of the doses described herein. In some variations, the therapeutic hydrogenated pyrido[4,3-b]indole or pharmaceutically acceptable salt thereof is dimebon.

In any of the above aspects or embodiments, the disease or indication is a neuronal or non-neuronal indication, such as Alzheimer's disease, impaired cognition associated with aging, age-associated hair loss, age-associated weight loss, age-associated vision disturbance, Huntington's disease, schizophrenia, canine cognitive dysfunction syndrome (CCDS), amyotrophic lateral sclerosis (ALS), Parkinson's disease, Lewy body disease, Menkes disease, Wilson disease, Creutzfeldt-Jakob disease, Fahr disease, an acute or chronic disorder involving cerebral circulation, such as stroke or cerebral hemorrhagic insult, age-associated memory impairment (AAMI), mild cognitive impairment (MCI), injury-related mild cognitive impairment (MCI), injury-related mild cognitive impairment (MCI) resulting from battlefield injuries, post-concussion syndrome, and adjuvant chemotherapy, neuronal death mediated ocular disease, macular degeneration, age-related macular degeneration, autism, including autism spectrum disorder, Asperger syndrome, and Rett syndrome, an avulsion injury, a spinal cord injury, myasthenia gravis, Guillain-Barré syndrome, multiple sclerosis, diabetic neuropathy, fibromyalgia, neuropathy associated with spinal cord injury, heart disease, diabetes, anorexia, AIDS- or chemotherapy-associated wasting, vascular injury, intestinal injury, cartilage injury, osteoarthritis, bacterial infection, viral infection, a first-, second-, or third-degree burn, a simple, compound, stress, or compression fracture, or a laceration.

In any of the above aspects or embodiments, the disease or condition is a neuronal indication such as Alzheimer's disease, impaired cognition associated with aging, age-associated hair loss, age-associated weight loss, age-associated vision disturbance, Huntington's disease, schizophrenia, canine cognitive dysfunction syndrome (CCDS), amyotrophic lateral sclerosis (ALS), Parkinson's disease, Lewy body disease, Menkes disease, Wilson disease, Creutzfeldt-Jakob disease, Fahr disease, an acute or chronic disorder involving cerebral circulation, such as stroke or cerebral hemorrhagic insult, age-associated memory impairment (AAMI), mild cognitive impairment (MCI), injury-related mild cognitive impairment (MCI), injury-related mild cognitive impairment (MCI) resulting from battlefield injuries, post-concussion syndrome, and adjuvant chemotherapy, neuronal death mediated ocular disorder, macular degeneration, age-related macular degeneration, autism, including autism spectrum disorder, Asperger syndrome, and Rett syndrome, an avulsion injury, a spinal cord injury, myasthenia gravis, Guillain-Barré syndrome, multiple sclerosis, diabetic neuropathy, fibromyalgia, or neuropathy associated with spinal cord injury.

In any of the above aspects or embodiments, the disease or condition is a neuronal indication, such as Alzheimer's disease, impaired cognition associated with aging, age-associated hair loss, age-associated weight loss, age-associated vision disturbance, Huntington's disease, schizophrenia, canine cognitive dysfunction syndrome (CCDS), amyotrophic lateral sclerosis (ALS), Parkinson's disease, Lewy body disease, Menkes disease, Wilson disease, Creutzfeldt-Jakob disease, Fahr disease, an acute or chronic disorder involving cerebral circulation, such as stroke or cerebral hemorrhagic insult, age-associated memory impairment (AAMI), or mild cognitive impairment (MCI). In any of the above aspects or embodiments, the disease or condition is not Alzheimer's disease. In any of the above aspects or embodiments, the disease or condition is not amyotrophic lateral sclerosis (ALS). In any of the above aspects or embodiments, the disease or condition is neither Alzheimer's disease nor amyotrophic lateral sclerosis (ALS). In any of the above aspects or embodiments, the disease or condition is not Huntington's disease. In any of the above aspects or embodiments, the disease or condition is not schizophrenia. In any of the above aspects or embodiments, the disease or condition is not MCI. In one variation, the individual is a human who has not been diagnosed with and/or is not considered at risk for developing Alzheimer's disease, Huntington's disease, amyotrophic lateral sclerosis, or schizophrenia. In one variation, the individual is a human who does not have impaired cognition associated with aging or does not have a non-life threatening condition associated with the aging process (such as loss of sight (cataract), deterioration of the dermatohairy integument (alopecia) or an age-associated decrease in weight due to the death of muscular and fatty cells) or a combination thereof. In any of the above aspects or embodiments, the disease or condition is a neuronal indication, such as injury-related mild cognitive impairment (MCI), injury-related mild cognitive impairment (MCI) resulting from battlefield injuries, post-concussion syndrome, and adjuvant chemotherapy, neuronal death mediated ocular disorder, macular degeneration, age-related macular degeneration, autism, including autism spectrum disorder, Asperger syndrome, and Rett syndrome, an avulsion injury, a spinal cord injury, myasthenia gravis, Guillain-Barré syndrome, multiple sclerosis, diabetic neuropathy, fibromyalgia, or neuropathy associated with spinal cord injury. In any of the above aspects or embodiments, the disease or condition is a non-neuronal indication, such as heart disease, diabetes, anorexia, AIDS- or chemotherapy-associated wasting, vascular injury, intestinal injury, cartilage injury, osteoarthritis, bacterial infection, viral infection, a first-, second-, or third-degree burn, a simple, compound, stress, or compression fracture, or a laceration

### Exemplary Cells and Methods

In one variation, the method involves administration of a therapy that contains a therapeutic compound, such as dimebon, and a cell, where the cell is an exemplary cell type as described in U.S. Pub. No. 2007/0110730, which is hereby incorporated by reference in its entirety. In some embodiments, the method involves incubating a cell with a therapeutic compound wherein the cell is an exemplary cell type as described in U.S. Pub. No. 2007/0110730. In some embodiments the cell that has been incubated with a therapeutic compound is administered to an individual in need thereof, such as an individual who has or is suspected of having a neuronal or non-neuronal indication. Any of the methods described herein can be used generate new cells to treat an injury or disease. In some embodiments, the cells are from tissues that have a high turnover rate or that are more likely to be subject to injury or disease, such as the epithelium or blood cells.

In some embodiments, the stem cells are multipotential cells that are capable of long-term self-renewal over the lifetime of a mammal. In some embodiments, stem cells may themselves be transplanted or, alternatively, they may be induced to produce differentiated cells (e.g., neurons, oligodendrocytes, Schwann cells, or astrocytes) for transplantation. Transplanted stem cells may also be used to express therapeutic molecules, such as growth factors, cytokines, anti-apoptotic proteins, and the like. Thus, stem cells are a potential source of cells for alternative treatments of diseases involving loss of cells or tissues.

In certain embodiments, the cells are capable of differentiating as dopaminergic neurons, and thus are a useful source of dopaminergic neurons for homotypic grafts into Parkinson's Disease patients. Other exemplary cells can differentiate as numerous mesodermal derivatives including smooth muscle cells, adipocytes, cartilage, bone, skeletal muscle, and cardiac muscle, and are expected to be capable of producing other mesodermal derivatives including kidney and hematopoietic cells. In some embodiments, the cells express markers of endodermal differentiation, and are expected to differentiate to cell types including pancreatic islet cells (e.g., α (alpha), β (beta), ψ (phi), δ (delta) cells), hepatocytes, and the like. In some embodiments, the cells are capable of differentiating to cells derived from all three germ layers. In some embodiments, the cells are used for autologous or heterologous transplants to treat, for example, other neurodegenerative diseases, disorders, or abnormal physical states.

In some embodiments, the cell(s) is the progeny of a multipotent stem cell purified from a peripheral tissue of a postnatal mammal. In some embodiments, the cell(s) is a mitotic cell or a differentiated cell (e.g., a neuron, an astrocyte, an oligodendrocyte, a Schwann cell, or a non-neural cell). Exemplary neurons include neurons expressing one or more of the following neurotransmitters: dopamine, GABA, glycine, acetylcholine, glutamate, and serotonin. Exemplary non-neural cells include cardiac muscle cells, pancreatic cells (e.g., islet cells (α (alpha), β (beta), ψ (phi), and δ (delta) cells), exocrine cells, endocrine cells, chondrocytes, osteocytes, skeletal muscle cells, smooth muscle cells, hepatocytes, hematopoietic cells, and adipocytes. These non-neural cell types include both mesodermal and endodermal derivatives. In an exemplary embodiment, the differentiated cells are purified.

In one aspect, the invention features a method of treating an individual having a disease associated with cell loss. In one embodiment, the method includes the step of transplanting cells such as multipotent stem cells into the region of the individual in which there is cell loss. In one embodiment, prior to the transplanting step, the method includes the steps of providing a culture of peripheral tissue and isolating a cell such as a multipotent stem cell from the peripheral tissue. The tissue may be derived from the same patient (autologous) or from either a genetically related or unrelated individual. After transplantation, the method may further include the step of differentiating (or allowing the differentiation of) the cell into a desired cell type to replace the cells that were lost. In some embodiments, the region is a region of the CNS or PNS, but can also be cardiac tissue, pancreatic tissue, or any other tissue in which cell transplantation therapy is possible. In another embodiment, the method includes the step of delivering the cells to the site of cell damage via the bloodstream, wherein the cells home to the site of cell damage. In one embodiment, the method for treating an individual includes the transplantation of the differentiated cells which are the progeny of stem cells.

Multipotent stem cells have tremendous capacity to differentiate into a range of neural and non-neural cell types. The non-neural cell types include both mesodermal and endodermal derivatives. In some embodiments, the cells are capable of differentiating to derivatives of all three germ layers. This capacity can be further influenced by modulating the culture conditions to influence the proliferation, differentiation, and survival of the cells. In one embodiment, modulating the culture conditions includes increasing or decreasing the serum concentration. In another embodiment, modulating the culture conditions includes increasing or decreasing the plating density. In still another embodiment, modulating the culture conditions includes the addition of one or more pharmacological agents to the culture medium. In another embodiment, modulating the culture conditions includes the addition of one or more therapeutic proteins (e.g., growth factors or anti-apoptotic proteins) to the culture medium. In each of the foregoing embodiments, pharmacological agents, therapeutic proteins, and small molecules can be administered individually or in any combination, and combinations of any of the pharmaceutical agents, therapeutic proteins, and small molecules can be co-administered or administered at different times.

In some embodiments, the cell is a purified multipotent stem cell from peripheral tissues of mammals, including skin, olfactory epithelium, and tongue. These cells proliferate in culture, so that large numbers of stem cells can be generated. These cells can be induced to differentiate, for example, into neurons, astrocytes, and/or oligodendrocytes by altering the culture conditions. They can also be induced to differentiate into non-neural cells such as smooth muscle cells, cartilage, bone, skeletal muscle, cardiac muscle, and adipocytes. The substantially purified neural stem cells are thus useful for generating cells for use, for example, in autologous transplants for the treatment of degenerative disorders or trauma (e.g., spinal cord injury). In one example, multipotent stem cells may be differentiated into dopaminergic neurons and implanted in the substantia nigra or striatum of a Parkinson's disease patient. In another example, the cells may be used to generate oligodendrocytes for use in autologous transplants for the treatment of multiple sclerosis. In another example, the multipotent stem cells may be used to generate Schwann cells for treatment of spinal cord injury, cardiac cells for the treatment of heart disease, or pancreatic islet cells for the treatment of diabetes. In some embodiments, the multipotent stem cells are used to generate adipocytes for the treatment of anorexia or wasting associated with many diseases including AIDS, cancer, and cancer treatments. In another example, multipotent stem cells may be used to generate smooth muscle cells to be used in vascular grafts. In another example, multipotent stem cells may be used to generate cartilage to be used to treat cartilage injuries and degenerative conditions of cartilage. In still another example, multipotent stem cells may be used to replace cells damaged or lost to bacterial or viral infection, or those lost to traumatic injuries such as burns, fractures, and lacerations.

If desired, the cells may be genetically modified to express, for example, a growth factor or an anti-apoptotic protein. Similarly, the proliferation, differentiation, or survival of the cells can be influenced by modulating the cell culture conditions including increasing or decreasing the concentration of serum in the culture medium and increasing or decreasing the plating density. In one embodiment, the cells are presorted prior to plating and differentiation such that only a sub-population of the cells are subjected to the differentiation conditions. Presorting of the cells can be done based on expression (or lack of expression) of a gene or protein, or based on differential cellular properties including adhesion and morphology.

The invention also features the use of the cells of this invention to introduce therapeutic compounds into the diseased, damaged, or physically abnormal CNS, PNS, or other tissue. Accordingly, the invention embraces a method of administering to an individual a therapy that contains a therapeutic compound, such as dimebon, and a cell, such as a cell associated with the CNS, PNS or other tissue. The invention also embraces a method of administering to an individual a cell, such as a cell associated with the CNS, PNS or other tissue that has been incubated with a therapeutic compound, such as dimebon. The cells thus act as vectors to transport the compound. In order to allow for expression of the therapeutic compound, suitable regulatory elements may be derived from a variety of sources, and may be readily selected by one with ordinary skill in the art. Examples of regulatory elements include a transcriptional promoter and enhancer or RNA polymerase binding sequence, and a ribosomal binding sequence, including a translation initiation signal. Additionally, depending on the vector employed, other genetic elements, such as selectable markers, may be incorporated into the recombinant molecule. The recombinant molecule may be introduced into the stem cells or the cells differentiated from the stem cells using in vitro delivery vehicles such as retroviral vectors, adenoviral vectors, DNA virus vectors, and liposomes. They may also be introduced into such cells in vivo using physical techniques such as microinjection and electroporation or chemical methods such as incorporation of DNA into liposomes. Such standard methods can be used to either transiently or stably introduce heterologous recombinant molecules into the cells. The genetically altered cells may be encapsulated in microspheres and implanted into or in proximity to the diseased or damaged tissue.

In one embodiment, the cells are used for the treatment of a neurological indication. In another aspect the cells such as multipotent stem cells are used as a source of non-neural cells, for example adipocytes, bone, cartilage, and smooth muscle cells. As an example, PCT publication WO99/16863 describes the differentiation of forebrain multipotent stem cells into cells of the hematopoietic cell lineage in vivo. Accordingly, the invention features methods of treating an individual having any disease or disorder characterized by cell loss by administering multipotent stem cells or cells derived from these cells to that patient and allowing the cells to differentiate to replace the cells lost in the disease or disorder. For example, transplantation of multipotent stem cells and their progeny provide an alternative to bone marrow and hematopoietic stem cell transplantation to treat blood-related disorders. Other uses of the multipotent stem cells are described in Ourednik et al. (Clin. Genet. 56:267-278, 1999), hereby incorporated by reference in its entirety. Multipotent stem cells and their progeny provide, for example, cultures of adipocytes and smooth muscle cells for study in vitro and for transplantation. Adipocytes secrete a variety of growth factors that may be desirable in treating cachexia, muscle wasting, and eating disorders. Smooth muscle cells may be, for example, incorporated into vascular grafts, intestinal grafts, etc. Cartilage cells have numerous orthopedic applications to treat cartilage injuries (e.g., sports injuries), as well as degenerative diseases and osteoarthritis. The cartilage cells can be used alone, or in combination with matrices well known in the art. Such matrices are used to mold the cartilage cells into requisite shapes.

### Therapeutic Compounds

When reference to organic residues or moieties having a specific number of carbons is made, unless clearly stated otherwise, it intends all geometric isomers thereof. For example, “butyl” includes n-butyl, sec-butyl, isobutyl and t-butyl; “propyl” includes n-propyl and isopropyl.

The term “alkyl” intends and includes linear, branched or cyclic hydrocarbon structures and combinations thereof. Preferred alkyl groups are those having 20 carbon atoms (C20) or fewer. More preferred alkyl groups are those having fewer than 15 or fewer than 10 or fewer than 8 carbon atoms.

The term “lower alkyl” refers to alkyl groups of from 1 to 5 carbon atoms. Examples of lower alkyl groups include methyl, ethyl, propyl, isopropyl, butyl, s- and t-butyl and the like. Lower alkyl is a subset of alkyl.

The term “aryl” refers to an unsaturated aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxain-3(4H)-one-7-yl), and the like. Preferred aryls includes phenyl and naphthyl.

The term “heteroaryl” refers to an aromatic carbocyclic group of from 2 to 10 carbon atoms and 1 to 4 heteroatoms selected from oxygen, nitrogen and sulfur within the ring. Such heteroaryl groups can have a single ring (e.g., pyridyl or furyl) or multiple condensed rings (e.g., indolizinyl or benzothienyl). Examples of heteroaryl residues include, e.g., imidazolyl, pyridinyl, indolyl, thiopheneyl, thiazolyl, furanyl, benzimidazolyl, quinolinyl, isoquinolinyl, pyrimidinyl, pyrazinyl, tetrazolyl and pyrazolyl.

The term “aralkyl” refers to a residue in which an aryl moiety is attached to the parent structure via an alkyl residue. Examples are benzyl, phenethyl and the like.

The term “heteroaralkyl” refers to a residue in which a heteroaryl moiety is attached to the parent structure via an alkyl residue. Examples include furanylmethyl, pyridinylmethyl, pyrimidinylethyl and the like.

The term “substituted heteroaralkyl” refers to heteroaryl groups which are substituted with from 1 to 3 substituents, such as residues selected from the group consisting of hydroxy, alkyl, alkoxy, alkenyl, alkynyl, amino, aryl, carboxyl, halo, nitro and amino.

The term “substituted aralkyl” refers to aralkyl groups which are substituted with from 1 to 3 substituents, such as residues selected from the group consisting of hydroxy, alkyl, alkoxy, alkenyl, alkynyl, amino, aryl, carboxyl, halo, nitro and amino.

The term “halo” or “halogen” refers to fluoro, chloro, bromo and iodo.

Therapeutic compounds for use in the methods, compositions, and kits described herein include hydrogenated pyrido[4,3-b]indoles or pharmaceutically acceptable salts thereof, such as an acid or base salt thereof. A hydrogenated pyrido[4,3-b]indole can be a tetrahydro pyrido[4,3-b]indole or pharmaceutically acceptable salt thereof. The hydrogenated pyrido[4,3-b]indole can also be a hexahydro pyrido[4,3-b]indole or pharmaceutically acceptable salt thereof. The hydrogenated pyrido[4,3-b]indole compounds can be substituted with 1 to 3 substituents, although unsubstituted hydrogenated pyrido[4,3-b]indole compounds or hydrogenated pyrido[4,3-b]indole compounds with more than 3 substituents are also contemplated. Suitable substituents include but are not limited to alkyl, lower alkyl, aralkyl, heteroaralkyl, substituted heteroaralkyl, substituted aralkyl, and halo.

Particular hydrogenated pyrido[4,3-b]indoles are exemplified by the Formulae A and B:

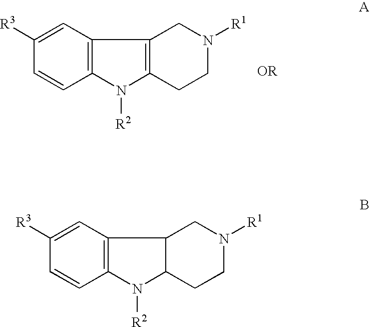

where R¹ is selected from the group consisting of alkyl, lower alkyl and aralkyl, R² is selected from the group consisting of hydrogen, aralkyl and substituted heteroaralkyl; and R³ is selected from the group consisting of hydrogen, alkyl, lower alkyl and halo.

In one variation, R¹ is alkyl, such as an alkyl selected from the group consisting of C₁-C₁₅alkyl, C₁₀-C₁₅alkyl, C₁-C₁₀alkyl, C₂-C₁₅alkyl, C₂-C₁₀alkyl, C₂-C₈alkyl, C₄-C₈alkyl, C₆-C₈alkyl, C₆-C₁₅alkyl, C₁₅-C₂₀alkyl; C₁-C₈alkyl and C₁-C₆alkyl. In one variation, R¹ is aralkyl. In one variation, R¹ is lower alkyl, such as a lower alkyl selected from the group consisting of C₁-C₂alkyl, C₁-C₄alkyl, C₂-C₄ alkyl, C₁-C₅ alkyl, C₁-C₃alkyl, and C₂-C₅alkyl.

In one variation, R¹ is a straight chain alkyl group. In one variation, R¹ is a branched alkyl group. In one variation, R¹ is a cyclic alkyl group.

In one variation, R¹ is methyl. In one variation, R¹ is ethyl. In one variation, R¹ is methyl or ethyl. In one variation, R¹ is methyl or an aralkyl group such as benzyl. In one variation, R¹ is ethyl or an aralkyl group such as benzyl.

In one variation, R¹ is an aralkyl group. In one variation, R¹ is an aralkyl group where any one of the alkyl or lower alkyl substituents listed in the preceding paragraphs is further substituted with an aryl group (e.g., Ar—C₁-C₆alkyl, Ar—C₁-C₃alkyl or Ar—C₁-C₁₅alkyl). In one variation, R¹ is an aralkyl group where any one of the alkyl or lower alkyl substituents listed in the preceding paragraphs is substituted with a single ring aryl residue. In one variation, R¹ is an aralkyl group where any one of the alkyl or lower alkyl substituents listed in the preceding paragraphs is further substituted with a phenyl group (e.g., Ph-C₁-C₆Alkyl or Ph-C₁-C₃Alkyl, Ph-C₁-C₁₅alkyl). In one variation, R¹ is benzyl.

All of the variations for R¹ are intended and hereby clearly described to be combined with any of the variations stated below for R² and R³ the same as if each and every combination of R¹, R² and R³ were specifically and individually listed.

In one variation, R² is H. In one variation, R² is an aralkyl group. In one variation, R² is a substituted heteroaralkyl group. In one variation, R² is hydrogen or an aralkyl group. In one variation, R² is hydrogen or a substituted heteroaralkyl group. In one variation, R² is an aralkyl group or a substituted heteroaralkyl group. In one variation, R² is selected from the group consisting of hydrogen, an aralkyl group and a substituted heteroaralkyl group.

In one variation, R² is an aralkyl group where R² can be any one of the aralkyl groups noted for R¹ above, the same as if each and every aralkyl variation listed for R¹ is separately and individually listed for R².

In one variation, R² is a substituted heteroaralkyl group, where the alkyl moiety of the heteroaralkyl can be any alkyl or lower alkyl group, such as those listed above for R¹. In one variation, R² is a substituted heteroaralkyl where the heteroaryl group is substituted with 1 to 3 C₁-C₃ alkyl substituents (e.g., 6-methyl-3-pyridylethyl). In one variation, R² is a substituted heteroaralkyl group wherein the heteroaryl group is substituted with 1 to 3 methyl groups. In one variation, R² is a substituted heteroaralkyl group wherein the heteroaryl group is substituted with one lower alkyl substituent. In one variation, R² is a substituted heteroaralkyl group wherein the heteroaryl group is substituted with one C₁-C₃ alkyl substituent. In one variation, R² is a substituted heteroaralkyl group wherein the heteroaryl group is substituted with one or two methyl groups. In one variation, R² is a substituted heteroaralkyl group wherein the heteroaryl group is substituted with one methyl group.

In other variations, R² is any one of the substituted heteroaralkyl groups in the immediately preceding paragraph where the heteroaryl moiety of the heteroaralkyl group is a single ring heteroaryl group. In other variations, R² is any one of the substituted heteroaralkyl groups in the immediately preceding paragraph where the heteroaryl moiety of the heteroaralkyl group is a multiple condensed ring heteroaryl group. In other variations, R² is any one of the substituted heteroaralkyl groups in the immediately preceding paragraph where the heteroaralkyl moiety is a pyridyl group (Py).

In one variation, R² is 6-CH₃-3-Py-(CH₂)₂—. An example of a compound containing this moiety is dimebon.

In one variation, R³ is hydrogen. In other variations, R³ is any one of the alkyl groups noted for R¹ above, the same as if each and every alkyl variation listed for R¹ is separately and individually listed for R³. In another variation, R³ is a halo group. In one variation, R³ is hydrogen or an alkyl group. In one variation, R³ is a halo or alkyl group. In one variation, R³ is hydrogen or a halo group. In one variation, R³ is selected from the group consisting of hydrogen, alkyl and halo. In one variation, R³ is Br. In one variation, R³ is I. In one variation, R³ is F. In one variation, R³ is Cl.

In a particular variation, the hydrogenated pyrido[4,3-b]indole is 2,8-dimethyl-5-(2-(6-methyl-3-pyridyl)ethyl)-2,3,4,5-tetrahydro-1H-pyrido[4,3-b]indole or a pharmaceutically acceptable salt thereof.

The hydrogenated pyrido[4,3-b]indoles can be in the form of pharmaceutically acceptable salts thereof, which are readily known to those of skill in the art. The pharmaceutically acceptable salts include pharmaceutically acceptable acid salts. Examples of particular pharmaceutically acceptable salts include hydrochloride salts or dihydrochloride salts. In a particular variation, the hydrogenated pyrido[4,3-b]indole is a pharmaceutically acceptable salt of 2,8-dimethyl-5-(2-(6-methyl-3-pyridyl)ethyl)-2,3,4,5-tetrahydro-1H-pyrido[4,3-b]indole, such as 2,8-dimethyl-5-(2-(6-methyl-3-pyridyl)ethyl)-2,3,4,5-tetrahydro-1H-pyrido[4,3-b]indole dihydrochloride (dimebon).

Particular hydrogenated pyrido[4,3-b]indoles can also be described by the Formula (1) or by the Formula (2):

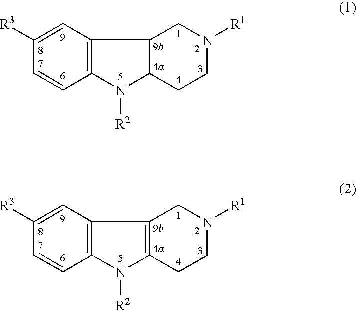

For compounds of a general Formula (1) or (2),

R¹ represents —CH₃, CH₃CH₂—, or PhCH₂— (benzyl);

### R² is —H, PhCH₂—, or 6CH₃-3-Py-(CH₂)₂—;

### R³ is —H, —CH₃, or —Br,

in any combination of the above substituents. All possible combinations of the substituents of Formula (1) and (2) are contemplated as specific and individual compounds the same as if each single and individual compound were listed by chemical name. Also contemplated are the compounds of Formula (1) or (2), with any deletion of one or more possible moieties from the substituent groups listed above: e.g., where R¹ represents —CH₃. In one variation, R² is —H, PhCH₂—, or 6CH₃-3-Py-(CH₂)₂—; and R³ is —H, —CH₃, or —Br, or where R¹ represents —CH₃; R² is 6CH₃-3-Py-(CH₂)₂—; and R³ represents —H, —CH₃, or —Br.

The above and any therapeutic compound herein may be in a form of salts with pharmaceutically acceptable acids and in a form of quaternized derivatives. Pharmaceutically acceptable salts refers to salts which retain the biological effectiveness and properties of the compound and which are not biologically or otherwise undesirable. In many cases, the compound will be capable of forming acid salts by virtue of an amino or other similar group. Pharmaceutically acceptable base addition salts can be prepared from inorganic and/or organic bases, where structure and functional groups permit. Pharmaceutically acceptable acid addition salts may be prepared from inorganic and/or organic acids. For example, inorganic acids include hydrochloric acid, dihydrochloric acid, hydrobromic acid, sulfuric acid, nitric acid, phosphoric acid, and the like. Organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. In one variation, the methods described employ compound (I) as a hydrochloride acid salt or a dihydrochloride acid salt.

The compound may be Formula (1), where R¹ is —CH₃, R² is —H, and R³ is —CH₃. The compound may be Formula (2), where R¹ is represented by —CH₃, CH₃CH₂—, or PhCH₂—; R² is —H, PhCH₂—, or 6CH₃-3-Py-(CH₂)₂—; R³ is —H, —CH₃, or —Br. The compound may be Formula (2), where R¹ is CH₃CH₂— or PhCH₂—, R² is —H, and R³ is —H; or a compound, where R¹ is —CH₃, R² is PhCH₂—, R³ is —CH₃; or a compound, where R¹ is —CH₃, R² is 6-CH₃-3-Py-(CH₂)₂—, and R³ is —CH₃; or a compound, where R¹ is —CH₃, R² is —H, R³ is —H or —CH₃; or a compound, where R¹ is —CH₃, R² is —H, R³ is —Br.

Compounds known from literature which can be used in the methods disclosed herein include the following specific compounds:
1. cis(±) 2,8-dimethyl-2,3,4,4a,5,9b-hexahydro-1H-pyrido[4,3-b]indole and its dihydrochloride;
2. 2-ethyl-2,3,4,5-tetrahydro-1H-pyrido[4,3-b]indole;
3. 2-benzyl-2,3,4,5-tetrahydro-1H-pyrido[4,3-b]indole;
4. 2,8-dimethyl-5-benzyl-2,3,4,5-tetrahydro-1H-pyrido[4,3-b]indole and its dihydrochloride;
5. 2-methyl-5-(2-methyl-3-pyridyl)ethyl-2,3,4,5-tetrahydro-1H-pyrido[4,3-b]indole and its sesquisulfate;
6. 2,8-dimethyl-5-(2-(6-methyl-3-pyridyl)ethyl)-2,3,4,5-tetrahydro-1H-pyrido[4,3-b]indole and its dihydrochloride (dimebon);
7. 2-methyl-2,3,4,5-tetrahydro-1H-pyrido[4,3-b]indole;
8. 2,8-dimethyl-2,3,4,5-tetrahydro-1H-pyrido[4,3-b]indole and its methyl iodide;
9. 2-methyl-8-bromo-2,3,4,5-tetrahydro-1H-pyrido[4,3-b]indole and its hydrochloride.

In one variation, the compound is of the Formula A or B and R¹ is selected from a lower alkyl or benzyl; R² is selected from a hydrogen, benzyl or 6-CH₃-3-Py-(CH₂)₂— and R³ is selected from hydrogen, lower alkyl or halo, or any pharmaceutically acceptable salt thereof. In another variation, R¹ is selected from —CH₃, CH₃CH₂—, or benzyl; R² is selected from —H, benzyl, or 6-CH₃-3-Py-(CH₂)₂—; and R³ is selected from —H, —CH₃ or —Br, or any pharmaceutically acceptable salt thereof. In another variation the compound is selected from the group consisting of: cis (±) 2,8-dimethyl-2,3,4,4a,5,9b-hexahydro-1H-pyrido[4,3-b]indole as a racemic mixture or in the substantially pure (+) or substantially pure (−) form; 2-ethyl-2,3,4,5-tetrahydro-1H-pyrido[4,3-b]indole; 2-benzyl-2,3,4,5-tetrahydro-1H-pyrido[4,3-b]indole; 2,8-dimethyl-5-benzyl-2,3,4,5-tetrahydro-1H-pyrido[4,3-b]indole; 2-methyl-5-(2-methyl-3-pyridyl)ethyl-2,3,4,5-tetrahydro-1H-pyrido[4,3-b]indole; 2,8-dimethyl-5-(2-(6-methyl-3-pyridyl)ethyl)-2,3,4,5-tetrahydro-1H-pyrido[4,3-b]indole; 2-methyl-2,3,4,5-tetrahydro-1H-pyrido[4,3-b]indole; 2,8-dimethyl-2,3,4,5-tetrahydro-1H-pyrido[4,3-b]indole; or 2-methyl-8-bromo-2,3,4,5-tetrahydro-1H-pyrido[4,3-b]indole or any pharmaceutically acceptable salt of any of the foregoing. In one variation, the compound is of the formula A or B wherein R¹ is —CH₃, R² is —H and R³ is —CH₃ or any pharmaceutically acceptable salt thereof. The compound may be of the Formula A or B where R¹CH₃CH₂— or benzyl, R² is —H, and R³ is —CH₃ or any pharmaceutically acceptable salt thereof. The compound may be of the Formula A or B where R¹ is —CH₃, R² is benzyl, and R³ is —CH₃ or any pharmaceutically acceptable salt thereof. The compound may be of the Formula A or B where R¹ is —CH₃, R² is 6-CH₃-3-Py-(CH₂)₂—, and R³ is —H or any pharmaceutically acceptable salt thereof. The compound may be of the Formula A or B where R² is 6-CH₃-3-Py-(CH₂)₂— or any pharmaceutically acceptable salt thereof. The compound may be of the Formula A or B where R¹ is —CH₃, R² is —H, and R³ is —H or —CH₃ or any pharmaceutically acceptable salt, thereof. The compound may be of the Formula A or B where R¹ is —CH₃, R² is —H, and R³ is —Br, or any pharmaceutically acceptable salt thereof. The compound may be of the Formula A or B where R¹ is selected from a lower alkyl or aralkyl, R² is selected from a hydrogen, aralkyl or substituted heteroaralkyl and R³ is selected from hydrogen, lower alkyl or halo.

The compound for use in the systems and methods may be 2,8-dimethyl-5-(2-(6-methyl-3-pyridyl)ethyl-2,3,4,5-tetrahydro-1H-pyrido[4,3-b]indole or any pharmaceutically acceptable salt thereof, such as an acid salt, a hydrochloride salt or a dihydrochloride salt thereof.

Any of the compounds disclosed herein having two stereocenters in the pyrido[4,3-b]indole ring structure (e.g., carbons 4a and 9b of compound (I)) includes compounds whose stereocenters are in a cis or a trans form. A composition may comprise such a compound in substantially pure form, such as a composition of substantially pure S,S or R,R or S,R or R,S compound. A composition of substantially pure compound means that the composition contains no more than 15% or no more than 10% or no more than 5% or no more than 3% or no more than 1% impurity of the compound in a different stereochemical form. For instance, a composition of substantially pure S,S compound means that the composition contains no more than 15% or no more than 10% or no more than 5% or no more than 3% or no more than 1% of the R,R or S,R or R,S form of the compound. A composition may contain the compound as mixtures of such stereoisomers, where the mixture may be enanteomers (e.g., S,S and R,R) or diastereomers (e.g., S,S and R,S or S,R) in equal or unequal amounts. A composition may contain the compound as a mixture of 2 or 3 or 4 such stereoisomers in any ratio of stereoisomers. Compounds disclosed herein having stereocenters other than in the pyrido[4,3-b]indole ring structure intends all stereochemical variations of such compounds, including but not limited to enantiomers and diastereomers in any ratio, and includes racemic and enantioenriched and other possible mixtures. Unless stereochemistry is explicitly indicated in a structure, the structure is intended to embrace all possible stereoisomers of the compound depicted.

Compound listed above as compounds 1-9 from the literature are detailed in the following publications. Synthesis and studies on neuroleptic properties for cis (±) 2,8-dimethyl-2,3,4,4a,5,9b-hexahydro-1H-pyrido[4,3-b]indole and its dihydrochloride are reported, for instance, in the following publication: Yakhontov, L. N., Glushkov, R. G., Synthetic therapeutic drugs. A. G. Natradze, Ed., Moscow Medicina, 1983, p. 234-237. Synthesis of compounds 2, 8, and 9 above, and data on their properties as serotonin antagonists are reported in, for instance, in C. J. Cattanach, A. Cohen & B. H. Brown, J. Chem. Soc. (Ser. C) 1968, p. 1235-1243. Synthesis of the compound 3 above is reported, for instance, in the article N. P. Buu-Hoi, O. Roussel, P. Jacquignon, J. Chem. Soc., 1964, N 2, p. 708-711. N. F. Kucherova and N. K. Kochetkov (General chemistry (Russ.), 1956, 26:3149-3154) describe the synthesis of the compound 4 above. Synthesis of compounds 5 and 6 above is described in the article by A. N. Kost, M. A. Yurovskaya, T. V. Mel'nikova, in Chemistry of heterocyclic compounds, 1973, N 2, p. 207-212. The synthesis of the compound 7 above is described by U, Horlein in Chem. Ber., 1954, Bd. 87, hft 4, 463-p. 472. M. Yurovskaya and I. L. Rodionov in Chemistry of heterocyclic compounds (1981, N 8, p. 1072-10).

### Additional Compositions of the Invention

In one aspect, the invention provides a pharmaceutical composition comprising: (a) a first therapy comprising a hydrogenated pyrido[4,3-b]indole or pharmaceutically acceptable salt in an amount sufficient to activate a cell, promote the differentiation of a cell, promote the proliferation of a cell, or any combination of two or more of the foregoing, and (b) a pharmaceutically acceptable carrier. In another aspect, the invention provides a pharmaceutical composition comprising: (a) a first therapy comprising a cell that has been incubated with a hydrogenated pyrido[4,3-b]indole or pharmaceutically acceptable salt thereof under conditions sufficient to activate the cell, promote the differentiation of the cell, promote the proliferation of the cell, or any combination of two or more of the foregoing, and (b) a pharmaceutically acceptable carrier.

In any of the above embodiments, the pharmaceutical composition further comprises a second therapy comprising a hydrogenated pyrido[4,3-b]indole or pharmaceutically acceptable salt thereof. In any of the above embodiments, the pharmaceutical composition further comprises a second therapy comprising a growth factor and/or anti-cell death compound. In any of the above embodiments, the pharmaceutical composition further comprises a second therapy comprising a hydrogenated pyrido[4,3-b]indole or pharmaceutically acceptable salt thereof and further comprising a third therapy comprising a growth factor and/or anti-cell death compound.

In one aspect, the invention provides a pharmaceutical composition comprising: (a) a first therapy comprising a hydrogenated pyrido[4,3-b]indole or pharmaceutically acceptable salt thereof, (b) a second therapy comprising a growth factor and/or anti-cell death compound, and (c) a pharmaceutically acceptable carrier. In one aspect, the invention provides a pharmaceutical composition comprising: (a) a first therapy comprising a cell, (b), a second therapy comprising a hydrogenated pyrido[4,3-b]indole or pharmaceutically acceptable salt thereof, and (c) a pharmaceutically acceptable carrier.

In any of the above embodiments, the pharmaceutical composition further comprises a third therapy comprising a growth factor and/or anti-cell death compound. In any of the above embodiments, the pharmaceutical composition comprises a cell type is selected from the group consisting of stem cells, neuronal stem cells, non-neuronal cells and neurons. In any of the above embodiments, the cell type is a neuronal stem cell or a neuronal cell, and wherein the pharmaceutical composition increases the length of one or more axons of the cell. In any of the above embodiments, the cell type is a neuronal stem cell, and the pharmaceutical composition promotes the differentiation of the neuronal stem cell into a neuronal cell. In any of the above embodiments, the neuronal stem cell differentiates into a hippocampal neuron, cortical neuron, or spinal motor neuron. In any of the above embodiments, the cell has not been incubated with a hydrogenated pyrido[4,3-b]indole or pharmaceutically acceptable salt thereof prior to administration to the individual.

In any of the above embodiments, the hydrogenated pyrido[4,3-b]indole is a tetrahydro pyrido[4,3-b]indole. In any of the above embodiments, the hydrogenated pyrido[4,3-b]indole is a hexahydro pyrido[4,3-b]indole. In any of the above embodiments, the hydrogenated pyrido[4,3-b]indole is of the formula:

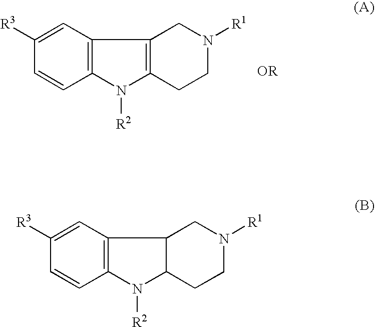

wherein R¹ is selected from a lower alkyl or aralkyl; R² is selected from a hydrogen, aralkyl or substituted heteroaralkyl; and R³ is selected from hydrogen, lower alkyl or halo. In any of the above embodiments, aralkyl is PhCH₂— and substituted heteroaralkyl is 6-CH₃-3-Py-(CH₂)₂—. In any of the above embodiments, R¹ is selected from CH₃—, CH₃CH₂—, or PhCH₂—; R² is selected from H—, PhCH₂—, or 6-CH₃-3-Py-(CH₂)₂—; and R³ is selected from H—, CH₃— or Br—. In any of the above embodiments, the hydrogenated pyrido[4,3-b]indole is selected from the group consisting of cis (±) 2,8-dimethyl-2,3,4,4a,5,9b-hexahydro-1H-pyrido[4,3-b]indole; 2-ethyl-2,3,4,5-tetrahydro-1H-pyrido[4,3-b]indole; 2-benzyl-2,3,4,5-tetrahydro-1H-pyrido[4,3-b]indole; 2,8-dimethyl-5-benzyl-2,3,4,5-tetrahydro-1H-pyrido[4,3-b]indole; 2-methyl-5-(2-methyl-3-pyridyl)ethyl-2,3,4,5-tetrahydro-1H-pyrido[4,3-b]indole; 2,8-dimethyl-5-(2-(6-methyl-3-pyridyl)ethyl)-2,3,4,5-tetrahydro-1H-pyrido[4,3-b]indole; 2-methyl-2,3,4,5-tetrahydro-1H-pyrido[4,3-b]indole; 2,8-dimethyl-2,3,4,5-tetrahydro-1H-pyrido[4,3-b]indole; 2-methyl-8-bromo-2,3,4,5-tetrahydro-1H-pyrido[4,3-b]indole. In any of the above embodiments, the hydrogenated pyrido[4,3-b]indole is 2,8-dimethyl-5-(2-(6-methyl-3-pyridyl)ethyl)-2,3,4,5-tetrahydro-1H-pyrido[4,3-b]indole. In any of the above embodiments, the pharmaceutically acceptable salt is a pharmaceutically acceptable acid salt. In any of the above embodiments, the pharmaceutically acceptable salt is a hydrochloride acid salt. In any of the above embodiments, the hydrogenated pyrido[4,3-b]indole is 2,8-dimethyl-5-(2-(6-methyl-3-pyridyl)ethyl)-2,3,4,5-tetrahydro-1H-pyrido[4,3-b]indole dihydrochloride.

In any of the above embodiments, R¹ is CH₃—, R² is H and R³ is CH₃—. In any of the above embodiments, R¹ CH₃CH₂— or PhCH₂—, R² is H—, and R³ is CH₃—. In any of the above embodiments, R¹ is CH₃—, R² is PhCH₂—, and R³ is CH₃—. In any of the above embodiments, R¹ is CH₃—, R² is 6-CH₃-3-Py-(CH₂)₂—, and R³ is H—. In any of the above embodiments, R² is 6-CH₃-3-Py-(CH₂)₂—. In any of the above embodiments, R¹ is CH₃—, R² is H—, and R³ is H— or CH₃—. In any of the above embodiments, R¹ is CH₃—, R² is H—, and R³ is Br—. In any of the above embodiments, the growth factor comprises VEGF, IGF-1, FGF, NGF, BDNF, GCS-F, GMCS-F, or any combination of two or more of the foregoing. In any of the above embodiments, the first and second therapies are administered sequentially. In any of the above embodiments, the first and second therapies are administered simultaneously. In any of the above embodiments, the first and second therapies are contained in the same container. In any of the above embodiments, the first and second therapies are contained in the separate containers. In any of the above embodiments, the first and second therapies have at least an additive effect. In any of the above embodiments, the first and second therapies have a synergistic effect.

### Compounds for Use in a Second or Additional Therapy

Where applicable, a method may employ (i) a therapeutic compound and/or a cell and (ii) one or more second or additional/subsequent therapies that are one or more growth factors and/or anti-cell death compounds.

### Growth Factors

Compounds for use in the methods, compositions, and kits described herein may include growth factors (e.g., vascular endothelial cell growth factors and/or trophic growth factors), fragments thereof, and compounds that mimic their effect. Examples of growth factors include NT-3, NT-4/5, HGF, CNTF, TGF-alpha, TGF-beta family members, neurotrophin-3, PDGF, GDNF (glial-derived neurotrophic factor), EGF family members, IGF, insulin, BMPs, Wnts, hedgehogs, heregulins, fragments thereof, and mimics thereof.

### Vascular Endothelial Cell Growth Factors

Compounds for use in the methods, compositions, and kits described herein may include vascular endothelial cell growth factors (VEGF), fragments thereof, and/or compound that mimic their effect. Exemplary VEGF molecules include VEGF121, VEGF145, VEGF165, VEGF189, VEGF206, other gene isoforms and fragments thereof (Sun F. Y., Guo X., “Molecular and cellular mechanisms of neuroprotection by vascular endothelial growth factor,” J. Neurosci. Res., 2005, 79(1-2):180-4). In some embodiments, the VEGF fragment contains at least 25, 50, 75, 100, 150 or 200 contiguous amino acids from a full-length VEGF protein and has at least 5%, 10%, 20%, 30%, 40%, 50%, 60%, 70%, 80%, 90% or 100% of an activity of a corresponding full-length VEGF protein.

### Trophic Growth Factors

Compounds for use in the methods, compositions, and kits described herein may include trophic growth factors (e.g., IGF-1, FGF (acidic and basic), NGF, BDNF, GCS-F and/or GMCS-F), fragments thereof, and compounds that mimic their effect. GCS-F and GMCS-F stimulate new neuron growth. Because trophic growth factors may stimulate cell growth, they are expected to improve, stabilize, eliminate, delay, or prevent a disease or condition or which the activation, differentiation, and/or proliferation of one or more cell types is beneficial. The combination of hydrogenated pyrido[4,3-b]indole such as dimebon and a trophic growth factor may reduce the apoptosis rate that is seen with new cell growth stimulation. An exemplary compound that mimics the effects of nerve growth factor is Xaliproden (Sanofi-Aventis) [SR 57746A, xaliprodene; Xaprila].

### Anti-Cell Death Compounds

Compounds for use in the methods, compositions, and kits described herein may include anti-cell death compounds (e.g., anti-apoptotic compounds). Exemplary anti-cell death compounds include anti-apoptotic compounds, such as IAP proteins, Bcl-2 proteins, Bcl-X_{L}, Trk receptors, Akt, PI3 kinase, Gab, Mek, E1B55K, Raf, Ras, PKC, PLC, FRS2, rAPs/SH2B, Np73, fragments thereof, and mimics thereof.

### Administration, Formulation, and Dosing of Therapies

Unless clearly indicated otherwise, the therapies (e.g., any of: (1) a therapeutic compound or pharmaceutically acceptable salt thereof, (2) a combination of (i) a therapeutic compound or pharmaceutically acceptable salt thereof and (ii) a growth factor and/or an anti-cell death compound, (3) a cell that has been incubated with a therapeutic compound or pharmaceutically acceptable salt thereof (4) a combination of (i) a therapeutic compound or pharmaceutically acceptable salt thereof and (ii) a cell that has been incubated with a therapeutic compound or pharmaceutically acceptable salt thereof, (5) a combination of (i) a therapeutic compound or pharmaceutically acceptable salt thereof, (ii) a cell that has been incubated with a therapeutic compound or pharmaceutically acceptable salt thereof, and (iii) a growth factor and/or an anti-cell death compound, (6) a combination of (i) a therapeutic compound or pharmaceutically acceptable salt thereof and (ii) a cell (such as a cell that has not been incubated with a therapeutic compound or pharmaceutically acceptable salt thereof), or (7) a combination of (i) a therapeutic compound or pharmaceutically acceptable salt thereof, (ii) a cell (such as a cell that has not been incubated with a therapeutic compound or pharmaceutically acceptable salt thereof), and (iii) a growth factor and/or an anti-cell death compound) for use herein, either as mono- or combination therapies, may be administered to the individual by any available dosage route and in any suitable dosage form. In one variation, the therapy is administered to the individual as a conventional immediate release dosage form. Where the therapy is a combination therapy, the invention also embraces administration of the therapy such that at least one component of the combination is administered to the individual as a conventional immediate release dosage form. In one variation, the therapy is administered to the individual as a sustained release form or part of a sustained release system, or as a controlled released form. Where the therapy is a combination therapy, the invention also embraces administration of the therapy such that at least one component of the combination is administered to the individual as a sustained release form or part of a sustained release system, or as a controlled release form.

A therapy as described above for use herein, such as any of therapies (1)-(7) described above, may be formulated for any available delivery route, whether immediate or sustained release, including an oral, mucosal (e.g., nasal, sublingual, vaginal, buccal or rectal), parenteral (e.g., intramuscular, intraperitoneal, subcutaneous, or intravenous), intrathecal, intraocular, topical or transdermal delivery form for delivery by the corresponding route. A therapy may be formulated with suitable carriers to provide delivery forms, which may be but are not required to be sustained release forms, that include, but are not limited to: tablets, caplets, capsules (such as hard gelatin capsules and soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs. The same or different routes of administration and delivery forms may be used for the components of a combination therapy.

In some embodiments, a dose of a therapy is administered once daily, twice daily, three times daily, or at higher frequencies. In some embodiments, a dose of a therapy is administered once a week, twice a week, three times a week, four times a week, or at higher frequencies. In some embodiments, a dose of a therapy is administered as a controlled release formulation every week, every two weeks, every three weeks, every four weeks, every five weeks, every six weeks, or at even longer intervals. In some embodiments, a dose (e.g., a dose for oral administration) of about 1 ng/day, 10 ng/day, 100 ng/day, 250 ng/day, 500 ng/day, 1 μg/day, 5 μg/day, 10 μg/day, 20 μg/day, 40 μg/day, 80 μg/day, 160 μg/day, 320 μg/day, or 120 mg/day of a therapeutic compound is administered. In some embodiments, the therapeutic compound is administered directly by infusion to the brain (e.g., intrathecal or intraventricular administration) at a dose of about 1 ng/day, 10 ng/day, 100 ng/day, 250 ng/day, 500 ng/day, 1 μg/day, 5 μg/day, 10 μg/day, 20 μg/day, 25 μg/day, 40 μg/day, 80 μg/day, 125 μg/day, 160 μg/day, 320 μg/day, or 120 mg/day. In some embodiments, a slow release pump or other device in the brain issued to administer any of the doses described herein.

Where applicable, the combined administration of one or more components of a combination therapy may include co-administration or concurrent administration of the combination components using separate formulations or a single pharmaceutical formulation or consecutive administration in any order. For some embodiments of concurrent administration, the administration of one component of a combination therapy overlaps the administration of another component of the combination therapy. In other embodiments, the administration of components of a combination therapy is non-concurrent. For example, in some embodiments, the administration of the therapeutic compound of a combination therapy is terminated before the other component of the therapy (such as a cell and/or a growth factor and/or an anti-cell death compound described herein) is administered. In some embodiments, the administration of the other component of the therapy is terminated before the therapeutic compound is administered. For sequential administration, there is preferably a time period while both (or all) components of a combination simultaneously exert their biological activities. Thus, a therapeutic compound may be administered prior to, during, or following administration of another component of a therapy. In various embodiments, the timing between at least one administration of a therapeutic compound and at least one administration of another component of a combination therapy is more than about 15 minutes, such as more than about any of 20, 30, 40, 50, or 60 minutes, or more than about any of 1 hour to about 24 hours, about 1 hour to about 48 hours, about 1 day to about 7 days, about 1 week to about 4 weeks, about 1 week to about 8 weeks, about 1 week to about 12 weeks, about 1 month to about 3 months, or about 1 month to about 6 months. In another embodiment, a therapeutic compound and another component of a combination therapy are administered concurrently to the individual in a single formulation or in separate formulations.

The amount of each therapy in a delivery form may be any effective amount. The amount each therapeutic compound contained in a therapy delivery form may be but is not limited to from about 10 ng to about 1,500 mg of therapeutic compound or more.

In one variation, a delivery form comprises an amount of therapeutic compound such that the daily dose of therapeutic compound is less than about 30 mg of compound. In some embodiments, a delivery form comprises a dose (e.g., a dose for oral administration) of about 1 ng/day, 10 ng/day, 100 ng/day, 250 ng/day, 500 ng/day, 1 μg/day, 5 μg/day, 10 μg/day, 20 μg/day, 25 μg/day, 40 μg/day, 80 μg/day, 125 μg/day, 160 μg/day, 320 μg/day, or 120 mg/day of a therapeutic compound. A treatment regimen involving a dosage form of a therapeutic compound alone or in a combination therapy, whether immediate release or a sustained release system, may involve administering a therapeutic compound to the individual in a dose of between about 0.1 and about 10 mg/kg of body weight, at least once a day and during the period of time required to achieve the therapeutic effect. In other variations, the daily dose (or other dosage frequency) of therapeutic compound as described herein is between about 0.1 and about 8 mg/kg; or between about 0.1 to about 6 mg/kg; or between about 0.1 and about 4 mg/kg; or between about 0.1 and about 2 mg/kg; or between about 0.1 and about 1 mg/kg; or between about 0.5 and about 10 mg/kg; or between about 1 and about 10 mg/kg; or between about 2 and about 10 mg/kg; or between about 4 to about 10 mg/kg; or between about 6 to about 10 mg/kg; or between about 8 to about 10 mg/kg; or between about 0.1 and about 5 mg/kg; or between about 0.1 and about 4 mg/kg; or between about 0.5 and about 5 mg/kg; or between about 1 and about 5 mg/kg; or between about 1 and about 4 mg/kg; or between about 2 and about 4 mg/kg; or between about 1 and about 3 mg/kg; or between about 1.5 and about 3 mg/kg; or between about 2 and about 3 mg/kg; or between about 0.001 and about 10 mg/kg; or between about 0.001 and about 4 mg/kg; or between about 0.001 and about 2 mg/kg; or between about 0.01 and about 10 mg/kg; or between about 0.01 and 4 mg/kg; or between about 0.01 mg/kg and 2 mg/kg; or between about 0.005 and about 10 mg/kg; or between about 0.005 and about 4 mg/kg; or between about 0.005 and about 3 mg/kg; or between about 0.005 and about 2 mg/kg; or between about 0.05 and 10 mg/kg; or between about 0.05 and 8 mg/kg; or between about 0.05 and 4 mg/kg; or between about 0.05 and 3 mg/kg; or between about 0.05 and about 2 mg/kg; or between about 10 kg to about 50 kg; or between about 10 to about 100 mg/kg or between about 10 to about 250 mg/kg; or between about 50 to about 100 mg/kg or between about 50 and 200 mg/kg; or between about 100 and about 200 mg/kg or between about 200 and about 500 mg/kg; or a dosage over about 100 mg/kg; or a dosage over about 500 mg/kg. In some embodiments, a daily dosage of a therapeutic compound, such as dimebon, is administered as a combination therapy with a second component that is a growth factor or an anti-cell death compound, such as a daily dosage of each administered therapeutic agent is less than about 0.1 mg/kg, which may include but is not limited to, a daily dosage of about 0.05 mg/kg, about 0.005 mg/kg, or about 0.001 mg/kg. Where the therapy contains a growth factor and/or an anti-cell death compound, the dosages above may apply to the growth factor and/or the anti-cell death compound as well as the therapeutic compound.

In some embodiments involving combination therapy (for both simultaneous and sequential administrations), the first therapy (e.g., a therapeutic compound such as dimebon) and a second therapy (e.g., a growth factor and/or anti-cell death compound and/or a cell) are administered at a predetermined ratio. For example, in some embodiments, the weight ratio of the first therapy (e.g., a therapeutic compound such as dimebon) to the second therapy is about 1 to 1. In some embodiments, the weight ratio may be between about 0.001 to about 1 and about 1000 to about 1, or between about 0.01 to about 1 and 100 to about 1. In some embodiments, the weight ratio of the first therapy (e.g., a therapeutic compound such as dimebon) to the second therapy is less than about any of 100:1, 50:1, 30:1, 10:1, 9:1, 8:1, 7:1, 6:1, 5:1, 4:1, 3:1, 2:1, and 1:1 In some embodiments, the weight ratio of the first therapy to the second therapy is more than about any of 1:1, 2:1, 3:1, 4:1, 5:1, 6:1, 7:1, 8:1, 9:1, 30:1, 50:1, 100:1. Other ratios are also contemplated.

A therapy, such as therapies (1)-(7) described herein above may be administered to an individual in accordance with an effective dosing regimen for a desired period of time or duration, such as at least about one month, at least about 2 months, at least about 3 months, at least about 6 months, or at least about 12 months or longer. In one variation, the therapy is administered on a daily or intermittent schedule for the duration of the individual's life. The components in a combination therapy may be administered for the same or different durations.

The dosing frequency for a therapy, such as therapies (1)-(7) described herein, including any combination disclosed herein, can be about a once weekly dosing. The dosing frequency can be about a once daily dosing. The dosing frequency can be more than about once weekly dosing. The dosing frequency can be less than three times a day dosing. The dosing frequency can be less than about three times a day dosing. The dosing frequency can be about three times a week dosing. The dosing frequency can be about a four times a week dosing. The dosing frequency can be about a two times a week dosing. The dosing frequency can be more than about once weekly dosing but less than about daily dosing. The dosing frequency can be about a once monthly dosing. The dosing frequency can be about a twice weekly dosing. The dosing frequency can be more than about once monthly dosing but less than about once weekly dosing. The dosing frequency can be intermittent (e.g., once daily dosing for 7 days followed by no doses for 7 days, repeated for any 14 day time period, such as about 2 months, about 4 months, about 6 months or more). The dosing frequency can be continuous (e.g., once weekly dosing for continuous weeks). Any of the dosing frequencies can employ any of the therapies described herein together with any of the dosages described herein, for example, the dosing frequency can be a once daily dosage of less than 0.1 mg/kg or less than about 0.05 mg/kg each of a therapeutic compound and a second or subsequent therapy that is a growth factor and/or anti-cell death compound and/or a cell.

The same or different dosing frequencies can be used for the components in a combination therapy. When administered separately, the therapeutic compound and a growth factor and/or anti-cell death compound and/or a cell can be administered at different dosing frequency or intervals. For example, the therapeutic compound can be administered weekly, while the growth factor and/or anti-cell death compound and/or a cell can be administered more or less frequently.

### Pharmaceutical Formulations

The therapies described herein, such as therapies (1)-(7) described herein, can be used in the preparation of a formulation, such as a pharmaceutical formulation, by combining the components of the therapy as an active ingredient with a pharmacologically acceptable carrier, which are known in the art. Depending on the therapeutic form of the system (e.g., transdermal patch vs. oral tablet), the carrier may be in various forms. In addition, pharmaceutical preparations may contain preservatives, solubilizers, stabilizers, re-wetting agents, emulgators, sweeteners, dyes, adjusters, salts for the adjustment of osmotic pressure, buffers, coating agents or antioxidants. In some embodiments, the pharmaceutical composition (e.g., composition containing cells) includes saline (such as saline buffered to pH=7.0), deionized water (such as deionized buffered to pH=7.0), or HEPES buffer (such as HEPES buffer at pH=7.0). Preparations comprising a combination therapy may also contain other substances which have valuable therapeutic properties. The components of a combination therapy can be prepared as part of the same or different formulations to be administered together or separately. Therapeutic forms may be represented by a usual standard dose and may be prepared by a known pharmaceutical method. Suitable doses of any of the co-administered components of a combination therapy may optionally be lowered due to the combined action (e.g., additive or synergistic effects) of the components. Suitable formulations can be found, e.g., in Remington's Pharmaceutical Sciences, Mack Publishing Company, Philadelphia, Pa., 20^{th} ed. (2000), which is incorporated herein by reference.

In one variation, the therapies (mono- or combination) are provided as a unit dosage form. The invention embraces unit dosage forms of any of therapies (1)-(7). In some embodiments in which the therapy calls for a cell and a therapeutic compound, one or more cells may be combined with a therapeutic compound (such as dimebon in saline) at a concentration ranging from about 1 pM to about 5 mM, from about 10 pM to about 500 μM, from about 50 pM to about 100 μM, from about 0.25 nM to about 20 μM, from about 1 nM to about 5 μM, from about 6 nM to about 800 nM, from about 30 nM to about 160 nM. In various embodiments for the ex vivo incubation of cells with a therapeutic compound, a therapeutic compound such as dimebon in saline is added to cells at a concentration of about 0.01 nM, 0.05 nM, 0.25 nM, 1.25 nM, 6.25 nM, 31.25 nM, 156.25 nM, 781 nM, 3.905 μM, 19.530 μM, 97.660 μM, or 488.280 μM.

### Kits

The invention further provides kits comprising: (a) a therapy as described herein, such as any of: (1) a therapeutic compound or pharmaceutically acceptable salt thereof, (2) a combination of (i) a therapeutic compound or pharmaceutically acceptable salt thereof and (ii) a growth factor and/or an anti-cell death compound, (3) a cell that has been incubated with a therapeutic compound or pharmaceutically acceptable salt thereof (4) a combination of (i) a therapeutic compound or pharmaceutically acceptable salt thereof and (ii) a cell that has been incubated with a therapeutic compound or pharmaceutically acceptable salt thereof, (5) a combination of (i) a therapeutic compound or pharmaceutically acceptable salt thereof, (ii) a cell that has been incubated with a therapeutic compound or pharmaceutically acceptable salt thereof, and (iii) a growth factor and/or an anti-cell death compound, (6) a combination of (i) a therapeutic compound or pharmaceutically acceptable salt thereof and (ii) a cell (such as a cell that has not been incubated with a therapeutic compound or pharmaceutically acceptable salt thereof), or (7) a combination of (i) a therapeutic compound or pharmaceutically acceptable salt thereof, (ii) a cell (such as a cell that has not been incubated with a therapeutic compound or pharmaceutically acceptable salt thereof), and (iii) a growth factor and/or an anti-cell death compound; and (b) instructions for use in treating, preventing, delaying the onset, and/or delaying the development of a disease or condition for which the activation, differentiation, and/or proliferation of one or more cell types is beneficial. The kits may employ any of the therapies disclosed herein, such as therapies (1)-(7) and instructions for use. In one variation, the kit employs one or more therapeutic compound, such as dimebon. The kits may be used for any one or more of the uses described herein, and, accordingly, may contain instructions for treating, preventing, delaying the onset, and/or delaying the development of a disease or condition for which the activation, differentiation, and/or proliferation of one or more cell types is beneficial, including but not limited to: a neuronal indication, a neurodegenerative disease, Alzheimer's disease, age-associated hair loss, age-associated weight loss, age-associated vision disturbance, Huntington's disease, schizophrenia, canine cognitive dysfunction syndrome (CCDS), neuronal death mediated ocular disease, macular degeneration, amyotrophic lateral sclerosis (ALS), Parkinson's disease, Lewy body disease, Menkes disease, Wilson disease, Creutzfeldt-Jakob disease, Fahr disease, acute or chronic disorders involving cerebral circulation, such as stroke, or cerebral hemorrhagic insult, age-associated memory impairment (AAMI) or mild cognitive impairment (MCI). In one variation, the kit employs dimebon. The therapies of the kit may be formulated in any acceptable form. For example, the compounds included in the kit may be supplied in buffered solution, as lyophilized powders, in single-use ampoules, and the like. In some embodiments, the kit contains a combination therapy where the components of the combination therapy are packaged together or separately, such as in separate containers, vials and the like.

In various embodiments, a kit includes a compound that increases the amount or activity of a growth factor (e.g., a VEGF protein or a trophic growth factor) and/or an anti-cell death compound. In some embodiments, one or more of these activities changes by at least or about 10%, 20%, 30%, 40%, 50%, 60%, 70%, 80%, 90%, 95% or 100% as compared to the corresponding activity in the same subject prior to treatment or compared to the corresponding activity in other subjects not receiving the combination therapy.

Kits generally comprise suitable packaging. The kits may comprise one or more containers comprising any compound described herein. Suitable packaging include, but is not limited to, vials, bottles, jars, flexible packaging (e.g., plastic bags), and the like. Each component (if there is more than one component) can be packaged in separate containers or some components can be combined in one container where cross-reactivity and shelf life permit. Kits may optionally provide additional components such as buffers.

The kits may optionally include a set of instructions, generally written instructions, although electronic storage media (e.g., magnetic diskette or optical disk) containing instructions are also acceptable, relating to the use of component(s) of the methods of the present invention (e.g., treating, preventing and/or delaying the onset and/or the development of a neuronal indication). The instructions included with the kit generally include information as to the components and their administration to an individual, such as information regarding dosage, dosing schedule, and route of administration.

The containers may be unit doses, bulk packages (e.g., multi-dose packages) or sub-unit doses. For example, kits may be provided that contain sufficient dosages of a therapeutic agent and/or a second compound that is a growth factor and/or an anti-cell death compound and/or a cell, to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of a therapy and instructions for use and be packaged in quantities sufficient for storage and use in pharmacies (e.g., hospital pharmacies and compounding pharmacies).

### Additional Kits of the Invention

In one aspect, the invention provides a kit comprising: (a) a first therapy comprising a hydrogenated pyrido[4,3-b]indole or pharmaceutically acceptable salt in an amount sufficient to activate a cell, promote the differentiation of a cell, promote the proliferation of a cell, or any combination of two or more of the foregoing, and (b) instructions for use of in the treatment, prevention, slowing the progression, delaying the onset, and/or delaying the development of a condition for which the activation, differentiation, and/or proliferation of one or more cell types is beneficial. In another aspect, the invention provides a kit comprising: (a) a first therapy comprising a cell that has been incubated with a hydrogenated pyrido[4,3-b]indole or pharmaceutically acceptable salt thereof under conditions sufficient to activate the cell, promote the differentiation of the cell, promote the proliferation of the cell, or any combination of two or more of the foregoing, and (b) instructions for use of in the treatment, prevention, slowing the progression, delaying the onset, and/or delaying the development of a condition for which the activation, differentiation, and/or proliferation of one or more cell types is beneficial. In one embodiment, the kit further comprises a second therapy comprising a hydrogenated pyrido[4,3-b]indole or pharmaceutically acceptable salt thereof. In one embodiment, the kit further comprises a second therapy comprising a growth factor and/or anti-cell death compound. In one embodiment, the kit further comprises a second therapy comprising a hydrogenated pyrido[4,3-b]indole or pharmaceutically acceptable salt thereof and further comprising a third therapy comprising a growth factor and/or anti-cell death compound.

In one aspect, the invention provides a kit comprising: (a) a first therapy comprising a hydrogenated pyrido[4,3-b]indole or pharmaceutically acceptable salt thereof, (b) a second therapy comprising a growth factor and/or anti-cell death compound, and (c) instructions for use of in the treatment, prevention, slowing the progression, delaying the onset, and/or delaying the development of a condition for which the activation, differentiation, and/or proliferation of one or more cell types is beneficial. In one aspect, the invention provides a kit comprising: (a) a first therapy comprising a cell, (b), a second therapy comprising a hydrogenated pyrido[4,3-b]indole or pharmaceutically acceptable salt thereof, and (c) instructions for use of in the treatment, prevention, slowing the progression, delaying the onset, and/or delaying the development of a condition for which the activation, differentiation, and/or proliferation of one or more cell types is beneficial. In one embodiment, the kit further comprises a third therapy comprising a growth factor and/or anti-cell death compound.

In any of the above embodiments, the cell type is selected from the group consisting of stem cells, neuronal stem cells, non-neuronal cell and neurons. In any of the above embodiments, the cell type is a neuronal stem cell or a neuronal cell, and wherein the first therapy and/or the second therapy increases the length of one or more axons of the cell. In any of the above embodiments, the cell type is a neuronal stem cell, and wherein the first therapy and/or the second therapy promotes the differentiation of the neuronal stem cell into a neuronal cell. In any of the above embodiments, the neuronal stem cell differentiates into a hippocampal neuron, cortical neuron, or spinal motor neuron. In any of the above embodiments, the cell has not been incubated with a hydrogenated pyrido[4,3-b]indole or pharmaceutically acceptable salt thereof prior to administration to the individual. In any of the above embodiments, the hydrogenated pyrido[4,3-b]indole is a tetrahydro pyrido[4,3-b]indole. In any of the above embodiments, the hydrogenated pyrido[4,3-b]indole is a hexahydro pyrido[4,3-b]indole. In any of the above embodiments, the hydrogenated pyrido[4,3-b]indole is of the formula:

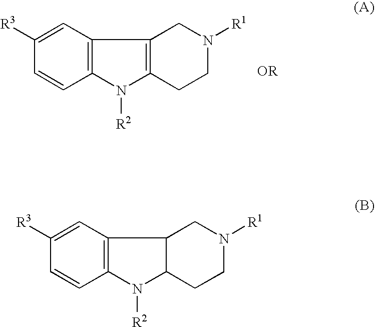

wherein R¹ is selected from a lower alkyl or aralkyl; R² is selected from a hydrogen, aralkyl or substituted heteroaralkyl; and R³ is selected from hydrogen, lower alkyl or halo. In any of the above embodiments, aralkyl is PhCH₂— and substituted heteroaralkyl is 6-CH₃-3-Py-(CH₂)₂—. In any of the above embodiments, R¹ is selected from CH₃—, CH₃CH₂—, or PhCH₂—; R² is selected from H—, PhCH₂—, or 6-CH₃-3-Py-(CH₂)₂—; and R³ is selected from H—, CH₃— or Br—. In any of the above embodiments, the hydrogenated pyrido[4,3-b]indole is selected from the group consisting of cis (±) 2,8-dimethyl-2,3,4,4a,5,9b-hexahydro-1H-pyrido[4,3-b]indole; 2-ethyl-2,3,4,5-tetrahydro-1H-pyrido[4,3-b]indole; 2-benzyl-2,3,4,5-tetrahydro-1H-pyrido[4,3-b]indole; 2,8-dimethyl-5-benzyl-2,3,4,5-tetrahydro-1H-pyrido[4,3-b]indole; 2-methyl-5-(2-methyl-3-pyridyl)ethyl-2,3,4,5-tetrahydro-1H-pyrido[4,3-b]indole; 2,8-dimethyl-5-(2-(6-methyl-3-pyridyl)ethyl)-2,3,4,5-tetrahydro-1H-pyrido[4,3-b]indole; 2-methyl-2,3,4,5-tetrahydro-1H-pyrido[4,3-b]indole; 2,8-dimethyl-2,3,4,5-tetrahydro-1H-pyrido[4,3-b]indole; and 2-methyl-8-bromo-2,3,4,5-tetrahydro-1H-pyrido[4,3-b]indole.

In any of the above embodiments, the hydrogenated pyrido[4,3-b]indole is 2,8-dimethyl-5-(2-(6-methyl-3-pyridyl)ethyl)-2,3,4,5-tetrahydro-1H-pyrido[4,3-b]indole. In any of the above embodiments, the pharmaceutically acceptable salt is a pharmaceutically acceptable acid salt. In any of the above embodiments, the pharmaceutically acceptable salt is a hydrochloride acid salt. In any of the above embodiments, the hydrogenated pyrido[4,3-b]indole is 2,8-dimethyl-5-(2-(6-methyl-3-pyridyl)ethyl)-2,3,4,5-tetrahydro-1H-pyrido[4,3-b]indole dihydrochloride. In any of the above embodiments, R¹ is CH₃—, R² is H and R³ is CH₃—. In any of the above embodiments, R¹CH₃CH₂— or PhCH₂—, R² is H—, and R³ is CH₃—. In any of the above embodiments, R¹ is CH₃—, R² is PhCH₂—, and R³ is CH₃—. In any of the above embodiments, R¹ is CH₃—, R² is 6-CH₃-3-Py-(CH₂)₂—, and R³ is H—. In any of the above embodiments, R² is 6-CH₃-3-Py-(CH₂)₂—. In any of the above embodiments, R¹ is CH₃—, R² is H—, and R³ is H— or CH₃—. In any of the above embodiments, R¹ is CH₃—, R² is H—, and R³ is Br—. In any of the above embodiments, the growth factor comprises VEGF, IGF-1, FGF, NGF, BDNF, GCS-F, GMCS-F, or any combination of two or more of the foregoing. In any of the above embodiments, the first and second therapies are administered sequentially. In any of the above embodiments, the first and second therapies are administered simultaneously. In any of the above embodiments, the first and second therapies are contained in the same pharmaceutical composition. In any of the above embodiments, the first and second therapies are contained in separate pharmaceutical compositions. In any of the above embodiments, the first and second therapies have at least an additive effect. In any of the above embodiments, the first and second therapies have a synergistic effect.

The following Examples are provided to illustrate but not limit the invention.

### EXAMPLES

### Example 1

### Increase in Neurite Outgrowth of Neurons that were Cultured with Dimebon

Dimebon, 2,8-dimethyl-5-(2-(6-methyl-3-pyridyl)-ethyl)-2,3,4,5-tetrahydro-1H-pyrido[4,3-b]indole dihydrochloride, was used as a representative compound of hydrogenated pyrido[4,3-b]indoles.

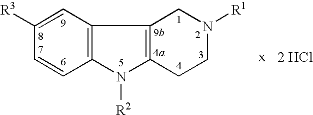

where R¹ and R³ are methyls, and

R² is 2-(6-methyl-3-pyridyl)-ethyl

Dimebon was tested to determine its ability to stimulate neurite outgrowth of cortical neurons, hippocampal neurons and spinal motor neurons. Similar methods may be used to test the ability of dimebon to stimulate neurite outgrowth in other types of neurons, such as hippocampal neurons.

Standard methods were used to isolate cortical neurons and spinal motor neurons. For the isolation of primary rat cortical neurons, the fetal brain from a pregnant rat at 17 days of gestation was prepared in Leibovitz's medium (L15; Gibco). The cortex was dissected out, and the meninges were removed. Trypsin (Gibco) was used to dissociate cortical neurons for 30 minutes at 37° C. with DNAse I. The cells were triturated in a 10 mL pipette in Dulbecco's Modified Eagle Media (“DMEM”; Gibco) with 10% Fetal Bovine Serum (“FBS”) (Gibco) and centrifuged at 350×g for 10 minutes at room temperature. The cells were suspended in Neurobasal medium supplemented with 2% B27 (Gibco) and 0.5 mM L-glutamine (Gibco). The cells were maintained at 30,000 cells per well of poly-L-lysine coated plates at 37° C. in 5% CO₂-95% air atmosphere. After adhesion, a vehicle control or dimebon was added at different concentrations to the medium. BDNF (50 ng/mL) was used as a positive control for neurite growth. After treatment, cultures were washed in phosphate-buffered saline (“PBS”; Gibco) and fixed in glutaraldehyde 2.5% in PBS. Cells were fixed after 3 days growth. Several pictures (˜80) of cells with neurites were taken per condition with a camera. The length measurements are made by analysis of the pictures using software from Image-Pro Plus (France). The results were expressed as mean (s.e.m.). Statistical analysis of the data was performed using one way analysis of variance (ANOVA).

To isolate hippocampal neurons, a female rat of 19 days gestation was killed by cervical dislocation, and the fetuses were removed from the uterus. Their brains were removed and placed in ice-cold medium of Leibovitz (L15, Gibco, Invitrogen). Meninges were carefully removed, and the hippocamps were dissected out. The hippocampal neurons were dissociated by trypsinization for 30 minutes at 37° C. (Trypsin-EDTA; Gibco) in the presence of DNAse I (Roche; Meylan). The reaction was stopped by the addition of DMEM (Gibco) cell culture medium with 10% of FBS (Gibco). The suspension was triturated with a 10-ml pipette using a needle syringe 21G and centrifuged at 350×g for 10 minutes at room temperature. The resulting pellet is resuspended in culture medium containing Neurobasal medium (Gibco) supplemented with 2% B27 supplement (Gibco) and 2 mM of glutamine (Gibco). Viable cells were counted in a Neubauer cytometer using the trypan blue exclusion test (Sigma) and seeded on the basis of 30,000 cells per Petri dish (Nunc) precoated with poly-L-lysine. Cells were allowed to adhere for two hours and maintained in a humidified incubator at 37° C. in 5% CO₂-95% air atmosphere. After adhesion, a vehicle control or dimebon was added at different concentrations to the medium. BDNF (1.85 nM) was used as a positive control for neurite growth. After treatment, cultures were washed in phosphate-buffered saline (PBS, Gibco) and fixed in glutaraldehyde 2.5% in PBS. Cells were fixed after 3 days growth. Several pictures (˜80) of cells with neurites without any branching were taken per condition with a camera (Coolpix 995; Nikon) fixed on microscope (Nikon, objective 40×). The length measurements were made by analysis of the pictures using software from Image-Pro Plus (France). The results were expressed as mean (s.e.m.). Statistical analysis of the data was performed using one way analysis of variance (ANOVA). Where applicable, Fisher's PLSD test was used for multiple pairwise comparison. The level of significance was set at p≦0.05.

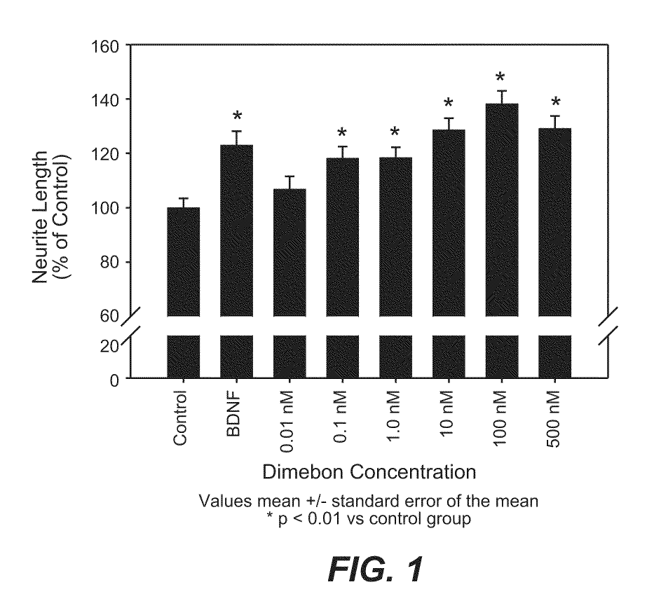

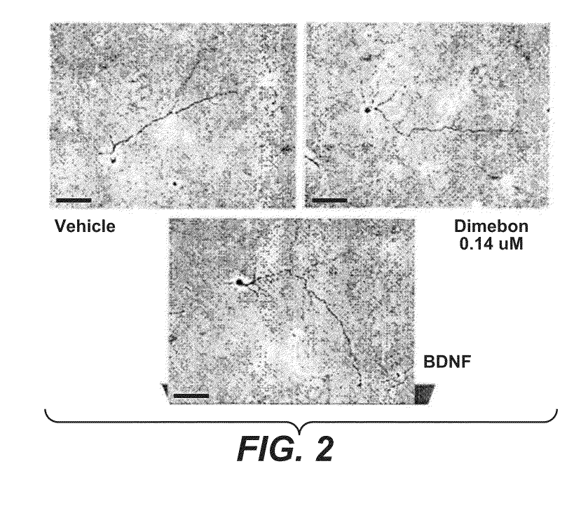

FIG. 1 is a Dimebon dose response curve for neurite outgrowth of primary rat cortical neurons. Low concentrations (i.e., picomolar (pM) and nanomolar (nM)) of Dimebon stimulated neurite outgrowth of primary rat cortical neurons. FIGS. 2A-2C are representative images of neurite outgrowth of primary rat cortical neurons treated with a vehicle control (saline) (FIG. 2A), 0.14 nM dimebon (FIG. 2B), or the positive control BDNF (FIG. 2C).

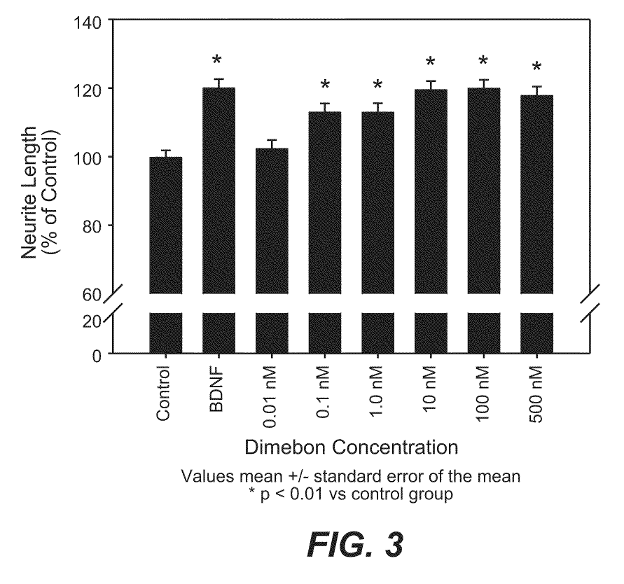

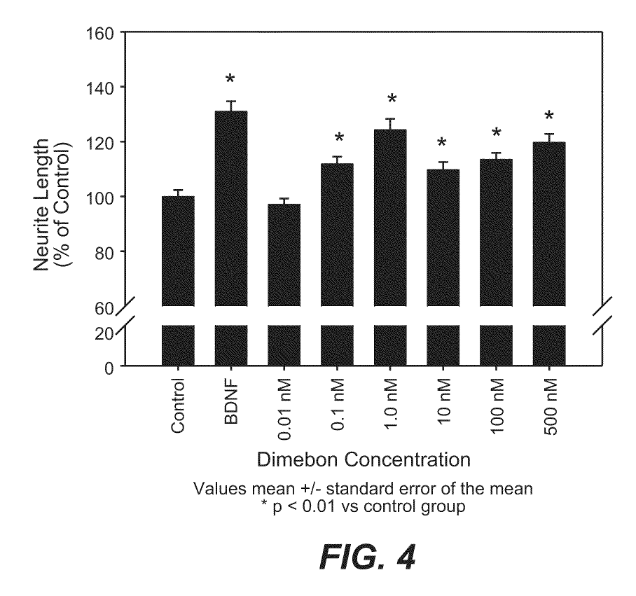

FIGS. 3 and 4 are dose response curves for neurite outgrowth of primary rat hippocampal neurons and primary rat spinal motor neurons, respectively. Picomolar and nanomolar concentrations of Dimebon stimulated neurite outgrowth in these neurons.

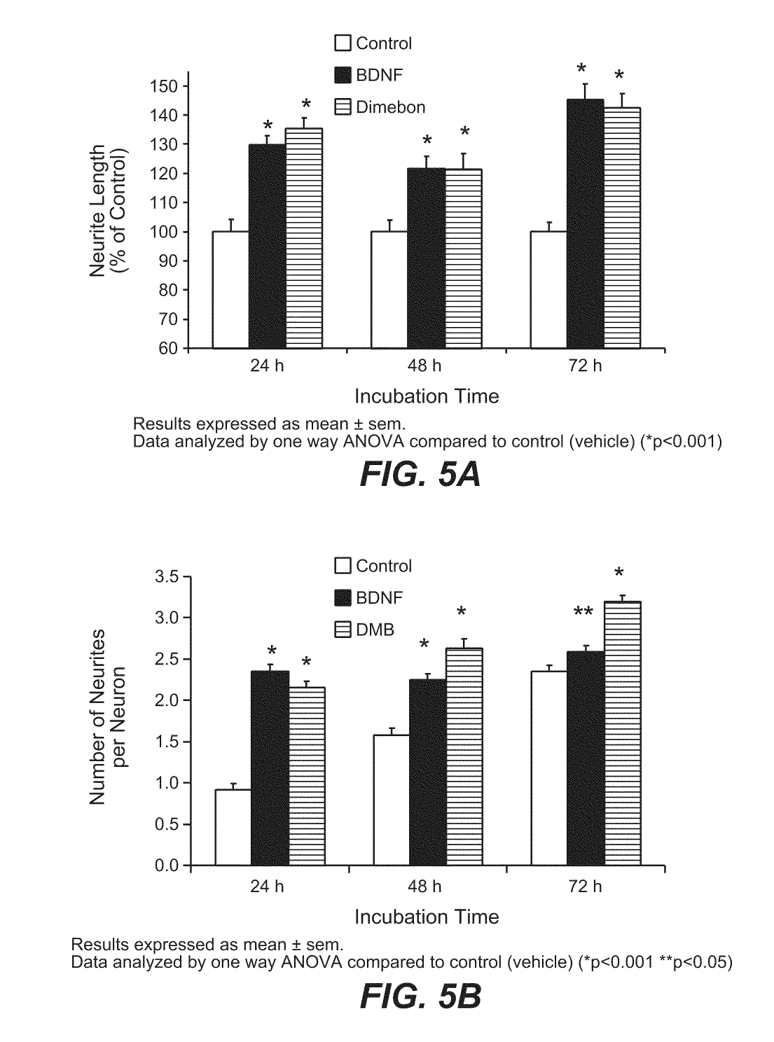

The effect of Dimebon (100 nM) on neurite outgrowth using primary hippocampal neurons was evaluated by measuring neurite length (expressed % of control, FIG. 5A) and number of neurites per neuron (FIG. 5B). The effects of vehicle, Dimebon and BDNF (50 ng/mL) were determined after incubations of 24 hours, 48 hours and 72 hours. Dimebon increased neurite length, and the number of neurites per neuron when compared to vehicle treatment. The effect of Dimebon on these endpoints was comparable to that obtained with BDNF.

### Example 2

### Increase in Neurogenesis in Rats Administered Dimebon

Dimebon, 2,8-dimethyl-5-(2-(6-methyl-3-pyridyl)-ethyl)-2,3,4,5-tetrahydro-1H-pyrido[4,3-b]indole dihydrochloride, was used as a representative compound of hydrogenated pyrido[4,3-b]indoles. Dimebon was tested to determine its ability to increase neurogenesis in vivo. In particular, the ability of dimebon to promote neurogenesis in the brain (such as hippocampal neurogenesis) of healthy rats was determined.

Wistar rats were obtained from Charles River or Harlan Winkelmann (Germany). Male rats were approximately 3 months old upon arrival at the animal colony. Animals were kept in an animal facility under standardized conditions and according to the animal welfare regulations of the Ministry of Science of the Austrian government. A record of bodyweights was maintained. The animals were allowed to acclimatize for at least one week prior to any experimental manipulations. Twelve rats per group were maintained on a 12 hour light/dark cycle. Three backup animals were maintained in order to compensate for animal loss. All rats were housed in groups of four per cage and had ad libitum access to food and water.

Rats were randomly allocated to four different treatment groups receiving intraperitoneal (i.p.) 5-bromo-2-deoxyuridine (BrdU, Sigma #B9285, 50 mg/kg body weight (b.w.)) and either (i) Dimebon at 10 mg/kg b.w./twice a day; (ii) Dimebon at 30 mg/kg b.w./twice a day; (iii) Dimebon at 60 mg/kg b.w./twice a day; or (iv) 0.2 mL vehicle (saline) twice a day. Treatment with BrdU, a synthetic nucleoside analog of thymidine, is commonly used to detect proliferating cells in living tissues such as the brain. Dimebon and vehicle were administered orally twice a day in a volume of 0.2 mL. BrdU was administered every other day. The daily Dimebon or vehicle treatment was performed several minutes before BrdU treatment. On day 14, animals were sacrificed approximately four hours after the last Dimebon treatment and one day after the last BrdU treatment. Diluted dimebon was prepared fresh daily.

At sacrifice, the rats were sedated using standard anesthesia. After transcardial perfusion with phosphate buffered saline (PBS) followed by 4% Paraformaldehyde/PBS, the brain from each rat was carefully removed, post-fixed in 4% Paraformaldehyde/PBS for one hour, transferred to 15% sucrose for cryoprotection, and shock-frozen in liquid isopentane. Brains were stored at −80° C. until cryo-cutting.

The brains were cut sagittally using a cryotome and stored at −20° C. until staining. Five layers were cut with 10 sections at 20 micrometers per layer with an interlayer slice gap of 100 micrometers. Standard Cresyl-Violett staining was performed on two consecutive slices per animal. BrdU immunohistochemistry was quantified to provide a morphological overview of cell division.

For the evaluation of BrdU positive cells/neurons, sections were processed by double-incubation with mouse anti-Neuronal Nuclei (NeuN) monoclonal antibody (Chemicon) and anti-BrdU (Abcam). One section per layer was treated in a three-day double-incubation with mouse anti-Neuronal Nuclei (NeuN) monoclonal antibody 1:800 (Chemicon, Hofheim. Germany) and anti-BrdU (sheep polyclonal to BrdU) 1:500 (Abcam, Cambridge, UK). The secondary antibodies were a Cy-3-conjugated pure affine goat anti-mouse IgG (H+L) 1:200 (Jackson ImmunoResearch, Cambridgeshire, UK) and a Cy 2-conjugated pure affine F(ab′)₂ fragment of donkey anti-sheep IgG (H+L) 1:100 (Jackson ImmunoResearch, Cambridgeshire, UK). Briefly, the anti-NeuN antibody was incubated overnight at 4° C., the Cy3 antibody was incubated the next day for one hour at room temperature, followed by the anti-BrdU antibody overnight at 4° C. and the Cy2 antibody for one hour at room temperature. To open the cell surfaces before the BrdU incubation, slices were treated with 2N HCl for 15 minutes at 40° C. and then washed for 20 minutes in a methanol mixture (60 ml methanol, 2 ml H₂O₂, and 0.6 ml Triton X) to block endogenous peroxidases. Niss1 staining was used as an overview staining.

Tiled images of the sagittal slice including the cortex and the hippocampus were recorded at 200-fold magnification. Each single image used a PCO PixelFly camera mounted on a NikonE800 microscope equipped with an software controlled (StagePro) automatic table. Both fluorescent colors, red for NeuN and green for BrdU, were recorded separately. For quantification, the images were merged. The evaluated variables included the region area, the absolute number of BrdU positive cells, the number of BrdU positive neurons, and the latter two values relative to the measured region area. Evaluations were concentrated on the whole hippocampus, especially the dentate gyrus and the subventricular zone.

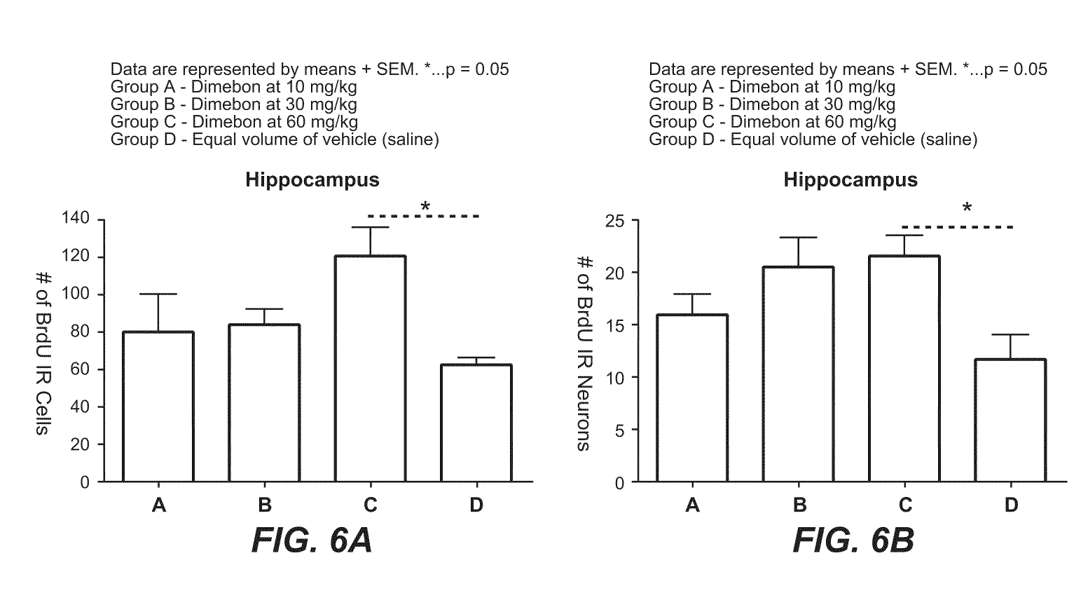

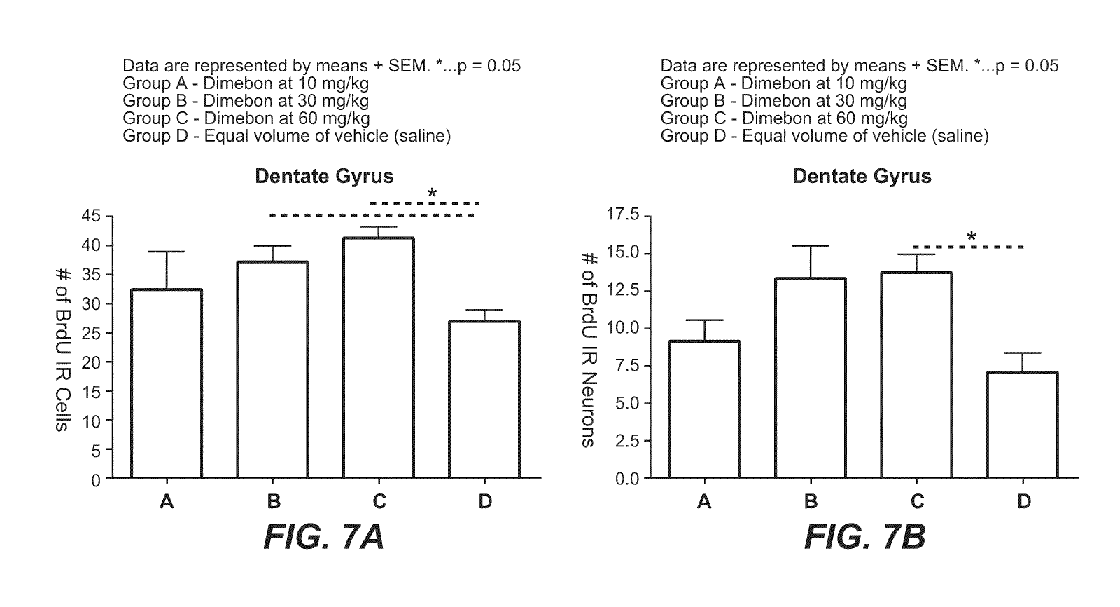

As illustrated in FIGS. 6A, 6B, 7A, and 7B, Dimebon treatment increases the total number of BrdU staining cells in the hippocampus and dentate gyms (FIGS. 6A and 7A, respectively), and increases the number of BrdU staining neurons in those same areas of the brain (FIGS. 6B and 7B).

### Example 3

### Determination of the Ability of Therapies of the Invention, Such as any of Therapies (1)-(7) to Inhibit Huntingtin-Induced Neurodegeneration of Photoreceptor Neurons in Drosophila Eyes

Therapies of the invention can be tested for their ability to inhibit mutant huntingtin-induced neurodegeneration of photoreceptor neurons in Drosophila eyes (which are reflective of neurodegenerative changes in fly brains). In particular, the insertion of the huntingtin gene responsible for Huntington's disease into the genomes of rodents and Drosophila fruit flies has been shown by others to induce many of the pathological and clinical signs of Huntington's disease seen in humans. Therefore, the study of these transgenic animals is useful to assess the pharmacological activities of potential Huntington's disease therapeutic agents prior to testing them in humans. Results in the described Drosophila model historically have correlated very well with transgenic mouse models for Huntington's disease. The close resemblance of the Drosophila model to the human Huntington's disease condition is described in J. L. Marsh et al., “Fly models of Huntington's Disease”, Hum. Mol. Genet., 2003, 12(review issue 2): R187-R193.

The Drosophila fruit fly is considered an excellent choice for modeling neurodegenerative diseases because it contains a fully functional nervous system with an architecture that separates specialized functions such as vision, smell, learning and memory in a manner not unlike that of mammalian nervous systems. Furthermore, the compound eye of the fruit fly is made up of hundreds of repeating constellations of specialized neurons which can be directly visualized through a microscope and upon which the ability of potential neuroprotective drugs to directly block neuronal cell death can easily be assessed. Finally, among human genes known to be associated with disease, approximately 75% have a Drosophila fruit fly counterpart.

In particular, the expression of mutant huntingtin protein in Drosophila fruit flies results in a fly phenotype that exhibits some of the features of human Huntington's disease. First, the presumed etiologic agent in Huntington's disease (mutant huntingtin protein) is encoded by a repeated triplet of nucleotides (CAG) which are called polyglutamine or polyQ repeats. In humans, the severity of Huntington's disease is correlated with the length of polyQ repeats. The same polyQ length dependency is seen in Drosophila. Secondly, no neurodegeneration is seen at early ages (early larval stages) in flies expressing the mutant huntingtin protein, although at later life stages (mature larval, pupal and aging adult stages), flies do develop the disease, similarly to humans, who generally manifest the first signs and symptoms of Huntington's disease starting in the fourth and fifth decades of life. Third, the neurodegeneration seen in flies expressing the mutant huntingtin gene is progressive, as it is in human patients with Huntington's disease. Fourth, the neuropathology in huntingtin-expressing flies leads to a loss of motor function as it does in similarly afflicted human patients. Last, flies expressing the mutant huntingtin protein die an early death, as do patients with Huntington's disease. For these reasons, therapies which show a neuroprotective effect in the Drosophila model of Huntington's disease are expected to be the most likely therapies to have a beneficial effect in humans.

For this assay, a therapy of the invention (e.g., a therapy that contains a therapeutic compound such as dimebon at a dose of, for example, 0, 1 μM, 5 μM, 10 μM, 100 μM, 100, 300 μM, or 1,000 μM) is administered to one group of transgenic Drosophila engineered to express the mutant huntingtin protein in all their neurons. This is accomplished by cloning a foreign gene into transposable p-element DNA vectors under control of a yeast upstream activator sequence that is activated by the yeast GAL4 transcription factor. These promoter fusions are injected into fly embryos to produce transgenic animals. The foreign gene is silent until crossed to another transgenic strain of flies expressing the GAL4 gene in a tissue specific manner. The Elav>Gal4 which expresses the transgene in all neurons from birth until death is used in the experiments described.

For therapy testing, 20-30 Httex1pQ93 virgins are mated to elav>Gal4 males and eggs are collected for about 20 hours at 25° C. and dispensed into vials (expected about 70% lethality from Htt effects). Upon eclosion, at least 80, 0-8 hour old flies are harvested and placed on or given a therapy of the invention, such as via a therapy-containing food (20 eclosed adults per vial) and scored when 7 days old. Therapy-containing food is prepared just before tester flies begin to emerge.

The two types of transgenic animals are crossed in order to collect enough closely age-matched controls to study. The crossed age-matched adults (about 20 per dosing group) are placed on therapy-containing food for 7 days. Animals are transferred to fresh food daily to minimize any effects caused by instability of the therapies. Survival is scored daily. The average number of photoreceptors at day zero is determined by scoring 7-10 of the newly eclosed tester siblings within six hours of eclosing. This establishes the baseline of degeneration at the time of exposure to therapy. At day 7, animals are sacrificed and the number of photoreceptor neurons surviving is counted. Scoring is by the pseudopupil method where individual functioning photoreceptors are revealed by light focused on the back of the head and visualized as focused points of light under a compound microscope focused at the photoreceptor level of the eye. For pseudopupil analysis, flies are decapitated and the heads are mounted in a drop of nail polish on a microscopic slide. The head is then covered with immersion oil and light is projected through the eye of the fly using a Nikon EFD-3/Optiphot-2 compound microscope with a 50× oil objective.

When multiple concentrations of therapy are tested (e.g., more than five concentrations of therapy), the test may be split into multiple days. This allows time for the pseudopupil analysis. Since a difference may be observed between Elav>Gal4;UAS>HttQ93 adult flies that emerge on different days, no therapy controls are set up for each day. To analyze the data, the non-treated adults are compared to the therapy treated adults that emerged on the same day.

### Example 4

### Determination of the Effect of Therapies of the Invention, Such as any of Therapies (1)-(7), on Motor Ability in a Drosophila Model

The effect of therapies of the invention on the motor function of Drosophila (obtained as described in the examples above) may be assessed by exploiting the strong negative geotropism of flies to climb upwards when they are tapped to the bottom of a vial. See, e.g., Le Bourg and Lint (1992) Hypergravity and aging in Drosophila melanogaster. 4. Climbing activity. Gerontol. 38:59-64. Animals are placed in a graduated vessel (e.g., a measuring cylinder). The distance climbed in 10 seconds is measured for each animal over 3 trials with a 5 minute rest period. In a separate experiment using tall thin plastic tubes rather than glass vials, the distance climbed in 30 seconds is also measured. Animals are scored for outcome without knowledge of treatment group.

Flies are tested for functional rescue using a behavior assay (climbing assay) where the distance climbed is measured. Flies are negatively geotropic and hence immediately climb up the wall of a container if tapped down to the bottom. In this assay, climbing is scored blind and each animal is given three trials that are then averaged. The climbing of 7 day old animals reared of food containing various concentrations of a therapy of the invention (e.g., a therapy containing 0, 10, 100 or 1,000 μM of therapeutic agent such as dimebon) is compared as is the climbing of animals on the day of eclosion. Two trials are performed. In the first, the ability to climb in large glass vials is monitored over 10 seconds. The second trial is similar to the first except that animals are tested in tall thin plastic tubes for climbing over 30 seconds.

### Example 5

### Determination of the Toxic Properties of a Therapy of the Invention, Such as any of Therapies (1)-(7), in Relation to Dopaminergic and GABAergic Neurons in Mesencephalic Cultures

Cell-based assays can be performed to determine the toxic properties of certain doses of the therapies described herein on dopaminergic and GABAergic neurons in mesencephalic cultures. Different concentrations of a therapy of the invention are added to the mesencephalic cultures, and the uptake of dopamine and GABA is assessed. This experiment establishes non-toxic doses of a therapy of the invention that can be used to test its effect on MPP+ toxicity as described in the following example.

Doses of a therapy of the invention ranging from 0 to 100 μM are tested using standard methods (see, e.g., W. Church and S. Hewett, J. Neurosci. Res., 73:811-817, 2003). The treatments are typically performed in triplicate. MPP+ may be used as a positive control.

### Example 6

### Determination of the Ability of Therapies of the Invention, Such as any of Therapies (1)-(7) to Protect Mesencephalic Cultures from Damage by MPP+

Mesencephalic cultures can be exposed to 1-methyl-4-phenylpyridine (“MPP+”) with and without a therapy described herein to evaluate whether the therapy counteracts MPP+-induced dopaminergic cell loss. In particular, mesencephalic cultures are pre-incubated for 24 hours in the presence of 1 or 5 μM of a therapy of the invention and then exposed to 1 μM MPP+ using standard methods (see, e.g., W. Church and S. Hewett, J. Neurosci. Res., 73:811-817, 2003). The treatments are typically done in triplicate. Dopamine and GABA uptake are measured as markers of respective cell viability. The experiment may also be performed by adding a milder dose of MPP+ (0.5 μM) to cultures pre-incubated with e.g., 1 μM therapy.

### Example 7

### Determination of the Ability of Therapies of the Invention, Such as any of Therapies (1)-(7) to Inhibit the Depletion of Dopamine and its Metabolites in a Mouse Model of 1-methyl-4-phenyl-1,2,3,6-tetrahydropyridine (“MPTP”)-Induced Nigrostriatal Degeneration

In vivo models of Parkinson's disease can also be used to determine the ability of any of the therapies described herein to treat, prevent, delay the onset, and/or delay the development of Parkinson's disease in mammals, such as humans. Several animal models of Parkinson's disease have been developed by others, such as those described in U.S. Pat. Nos. 6,878,858; 5,853,385; 7,105,504; and 7,037,657. Other useful models include models of nigrostriatal degeneration (e.g., paraquat-induced nigral cell loss; see, e.g., Amy Manning-Bog et al., J. Neurosci., 23(8):3095-3099, 2003) and/or other paradigms of toxicant-induced nigrostriatal damage (e.g., chronic MPTP exposure).

In one method, a mouse model of MPTP-induced nigrostriatal degeneration is used to analyze the ability of a therapy described herein to treat, prevent, delay the onset, and/or delay the development of Parkinson's disease. In particular, measurements are taken of the ability a therapy of the invention to prevent the depletion of dopamine and its compounds (DOPAC and HVA) in the mouse striatum that is caused by MPTP.

Specifically, a therapy of the invention is administered before, at the time of and after MPTP exposure. Animals receive two intraperitoneal injections of therapy at 9:00 a.m. and 4:00 p.m. for two days prior to MPTP. On the day of MPTP administration, mice are injected with therapy at 9:00 a.m., followed by MPTP at 1:00 p.m. and therapy again at 4:00 p.m. Finally, two daily doses of therapy are given to mice for six days after MPTP exposure. MPTP is injected subcutaneously at a dose of 30 mg/kg. Control animals received vehicle instead of a therapy of the invention and saline instead of MPTP. Animals are sacrificed by cervical dislocation on day 7 after MPTP exposure. Exemplary treatment groups are summarized below.

| Treatment groups | N |
|---|---|
| 1. Control (vehicle only) | 6 |
| 2. A therapy of the invention (10 mg/kg × 2/day, i.p.) | 7 |
| 3. MPTP (30 mg/kg, s.c.) | 7 |
| 4. MPTP (30 mg/kg, s.c.) + a therapy of the invention | 8 |
| (1 mg/kg × 2/day, i.p.) | |
| 5. MPTP (30 mg/kg, s.c.) + a therapy of the invention | 8 |
| (10 mg/kg × 2/day, i.p.) | |
| Total C57BL/6 mice (age 8 weeks) | 36 |

At the end of the experiment, the mice are sacrificed, and the striata (left and right) are dissected on ice. The left striatum is immediately placed in ice-cold 0.4 M perchloric acid and processed for assays of DA, DOPAC and HVA. The right striatum as well as midbrain blocks are also dissected and stored for potential future use (e.g., measurements of tyrosine hydroxylase levels in the striatal samples by Western, measurements of dopamine transporter binding in the striatal samples and/or stereological counting of dopaminergic neurons in the substantia nigra may be later performed if desired). DA, DOPAC and HVA are measured by HPLC with electrochemical detection following methods previously described (Purisai et al., Neurobiol. Dis. 20:898-906, 2005).

The neuroprotective effects of a therapy of the invention may also be tested in this protocol at lower doses, including 0.01 mg/kg, 0.05 mg/kg, 0.10 mg/kg, 1 mg/kg, and 5 mg/kg, or using other models of nigrostriatal degeneration (e.g., paraquat-induced nigral cell loss) and/or other paradigms of toxicant-induced nigrostriatal damage (e.g., chronic MPTP exposure).

### Example 8

### Use of an In Vivo Model to Determine the Ability of Therapies of the Invention, Such as Therapies (1)-(7) to Treat, Prevent and/or Delay the Onset and/or the Development of Alzheimer's Disease

In vivo models of Alzheimer's disease can also be used to determine the ability of any of the therapies described herein to treat, prevent and/or delay the onset and/or the development of Alzheimer's disease in mammals, such as humans. An exemplary animal model of Alzheimer's disease includes transgenic mice over-expressing the ‘Swedish’ mutant amyloid precursor protein (APP; Tg2576; K670N/M671L; Hsiao et al., 1996, Science, 274:99-102). The phenotype present in these mice has been well-characterized (Holcomb L. A. et al., 1998, Nat. Med., 4:97-100; Holcomb L. A. et al., 1999, Behav. Gen., 29:177-185; and McGowan E., 1999, Neurobiol. Dis., 6:231-244). The neuroprotective effects of a therapy of the invention may also be tested in this model at lower doses, including 0.01 mg/kg, 0.05 mg/kg, 0.10 mg/kg, 1 mg/kg, and 5 mg/kg, or using other animal models of Alzheimer's disease. Standard methods can be used to determine whether any of the therapies of the invention decrease the amount of Aβ deposits in the brains of these mice (see, e.g., WO 2004/032868, published Apr. 22, 2004).

### Example 9

### Use of an In Vitro Model to Determine the Ability of Therapies of the Invention, Such as Therapies (1)-(7) to Treat, Prevent and/or Delay the Onset and/or the Development of Amyotrophic Lateral Sclerosis

In vitro models of ALS can be used to determine the ability of any of the therapies described herein to reduce cell toxicity that is induced by a SOD1 mutation. A reduction in cell toxicity is indicative of the ability to treat, prevent and/or delay the onset and/or the development of ALS in mammals, such as humans.

In one exemplary in vitro model of ALS, N2a cells (e.g., the mouse neuroblastoma cell cline N2a sold by InPro Biotechnology, South San Francisco, Calif., USA) are transiently transfected with a mutant SOD1 in the presence or absence of various concentrations of a therapy of the invention. Standard methods can be used for this transfection, such as those described by Y. Wang et al., (J. Nucl. Med., 46(4):667-674, 2005). Cell toxicity can be measured using any routine method, such as cell counting, immunostaining, and/or MTT assays to determine whether the therapy attenuates mutant SOD1-mediated toxicity in N2a cells (see, e.g., U.S. Pat. No. 7,030,126; Y. Zhang et al., Proc. Natl. Acad. Sci. USA, 99(11):7408-7413, 2002; or S. Fernaeus et al., Neurosci Letts. 389(3):133-6, 2005).

### Example 10

### Use of an In Vivo Model to Determine the Ability of Therapies of the Invention, Such as Therapies (1)-(7) to Treat, Prevent and/or Delay the Onset and/or the Development of Amyotrophic Lateral Sclerosis

In vivo models of ALS can also be used to determine the ability of any of the therapies described herein to treat, prevent and/or delay the onset and/or the development of ALS in mammals, such as humans. Several animal models of ALS or motor neuron degeneration have been developed by others, such as those described in U.S. Pat. Nos. 7,030,126 and 6,723,315.

For example, several lines of transgenic mice expressing mutated forms of SOD responsible for the familial forms of ALS have been constructed as murine models of ALS (U.S. Pat. No. 6,723,315). Transgenic mice overexpressing mutated human SOD carrying a substitution of glycine 93 by alanine (FALS_{G93A} mice) have a progressive motor neuron degeneration expressing itself by a paralysis of the limbs, and die at the age of 4-6 months (Gurney et al., Science, 264, 1772-1775, 1994). The first clinical signs consist of a trembling of the limbs at approximately 90 days, then a reduction in the length of the step at 125 days. At the histological level, vacuoles of mitochondrial origin can be observed in the motor neurons from approximately 37 days, and a motor neurons loss can be observed from 90 days. Attacks on the myelinated axons are observed principally in the ventral marrow and a little in the dorsal region. Compensatory collateral reinnervation phenomena are observed at the level of the motor plaques.

FALS_{G93A} mice constitute a very good animal model for the study of the physiopathological mechanisms of ALS as well as for the development of therapeutic strategies. These mice exhibit a large number of histopathological and electromyographic characteristics of ALS. The electromyographic performances of the FALS_{G93A} mice indicate that they fulfill many of the criteria for ALS: (1) reduction in the number of motor units with a concomitant collateral reinnervation, (2) presence of spontaneous denervation activity (fibrillations) and of fasciculation in the hind and fore limbs, (3) modification of the speed of motor conduction correlated with a reduction in the motor response evoked, and (4) no sensory attack. Moreover, facial nerve attacks are rare, even in the aged FALS_{G93A} mice, which is also the case in patients. The FALS_{G93A} mice are available from Transgenic Alliance (L'Arbresle, France). Additionally, heterozygous transgenic mice carrying the human SOD1 (G93A) gene can be obtained from the Jackson Laboratory (Bar Harbor, Me., USA) (U.S. Pat. No. 7,030,126). These mice have 25 copies of the human G93A SOD mutation that are driven by the endogenous promoter. Survival in the mouse is copy number dependent. Mouse heterozygotes developing the disease can be identified by PCR after taking a piece of tail and extracting DNA.

Other animal models having motor neuron degeneration exist (U.S. Pat. No. 6,723,315; Sillevis-Smitt & De Jong, J. Neurol. Sci., 91, 231-258, 1989; Price et al., Neurobiol. Disease, 1, 3-11, 11994), either following an acute neurotoxic lesion (treatment with IDPN, with excitotoxins) or due to a genetic fault (wobbler, pmn, Mnd mice or HCSMA Dog). Among the genetic models, the pmn mice are particularly well-characterized on the clinical, histological and electromyographic level. The pmn mutation is transmitted in the autosomal recessive mode and has been localized on chromosome 13. The homozygous pmn mice develop a muscular atrophy and paralysis which is manifested in the rear members from the age of two to three weeks. All the non-treated pmn mice die before six to seven weeks of age. The degeneration of their motor neurons begins at the level of the nerve endings and ends in a massive loss of myelinized fibres in the motor nerves and especially in the phrenic nerve which ensures the inervation of the diaphragm. Contrary to the FALS_{G93A} mouse, this muscular denervation is very rapid and is virtually unaccompanied by signs of reinervation by regrowth of axonal collaterals. On the electromyographic level, the process of muscular denervation is characterized by the appearance of fibrillations and by a significant reduction in the amplitude of the muscular response caused after supramaximal electric stimulation of the nerve.

A line of Xt/pmn transgenic mice has also been used previously as another murine model of ALS (U.S. Pat. No. 6,723,315). These mice are obtained by a first crossing between C57/B156 or DBA2 female mice and Xt pmn⁺/Xt⁺ pmn male mice (strain 129), followed by a second between descendants Xt pmn⁺/Xt⁺ pmn⁺ heterozygous females (N1) with initial males. Among the descendant mice (N2), the Xt pmn⁺/Xt⁺ pmn double heterozygotes (called “Xt pmn mice”) carrying an Xt allele (demonstrated by the Extra digit phenotype) and a pan allele (determined by PCR) are chosen for the future crossings.

In one exemplary method for testing the activity of a therapy described herein in an in vivo model of ALS, female mice (B6SJL) are purchased to breed with the transgenic males that overexpress a mutated SOD carrying a substitution of glycine 93 by alanine (e.g., FALS_{G93A} mice). Two females are put in each cage with one male and monitored at least daily for pregnancy. As each pregnant female is identified, it is removed from the cage and a new non-pregnant female is added. Since 40-50% of the pups are expected to be transgenic, a colony of, for example, at least 200 pups can be born at approximately the same time. After genotyping at three weeks of age, the transgenic pups are weaned and separated into different cages by sex.

At least 80 transgenic mice (both male and female) are randomized into four groups: 1) vehicle treated (20 mice), 2) dose 1 (3 mg/kg/day; 20 mice), 3) dose 2 (10 mg/kg/day; 20 mice) and 3) dose 3 (30 mg/kg/day; 20 mice). Mice are evaluated daily. This evaluation includes analysis of weight, appearance (fur coat, activities, etc.) and motor coordination. Treatment starts at approximate stage 3 and continues until mice are euthanized. In one aspect, a therapy of the invention being tested is administered to the mice in their food. The neuroprotective effects of a therapy of the invention may also be tested in this protocol at lower doses, including 0.01 mg/kg, 0.05 mg/kg, 0.10 mg/kg, 1 mg/kg, and 5 mg/kg, or using other models of ALS.

The onset of clinical disease is scored by examining the mouse for tremor of its limbs and for muscle strength. The mice are lifted gently by the base of the tail and any muscle tremors are noted, and the hind limb extension is measured. Muscle weakness is reflected in the inability of the mouse to extend its hind limbs. The mice are scored on a five point scale for symptoms of motor neuron dysfunction: 5—no symptoms; 4—weakness in one or more limbs; 3—limping in one or more limbs; 2—paralysis in one or more limbs; 1-animal negative for reflexes, unable to right itself when placed on its back.

In animals showing signs of paralysis, moistened food pellets are placed inside the cage. When the mice are unable to reach food pellets, nutritional supplements are administered through assisted feeding (Ensure®, p.o., twice daily). Normal saline is supplemented by i.p. administration, 1 ml twice daily if necessary. In addition, these mice are weighed daily. If necessary, mice are cleaned by the research personnel, and the cage bedding is changed frequently. At end-stage disease, mice lay on their sides in their cage. Mice are euthanized immediately if they cannot right themselves within 10 seconds, or if they lose 20% of their body weight.

Spinal cords are collected from the fourth, eighth, twelfth, sixteenth and twentieth animal euthanized in each treatment group (total of five animals per treatment group, twenty animals total). These spinal cords are analyzed for mutant SOD1 content in mitochondria using standard methods (see, e.g., J. Liu et al., Neuron, 43(1):5-17, 2004).

If desired, the effect of a therapy of the invention in the ALS mouse model can be further characterized using standard methods to measure the size of the bicep muscles, the muscle morphology, the muscle response to electric stimulation, the number of spinal motor neurons, muscle function, and/or the amount of oxidative damage, e.g., as described in U.S. Pat. No. 6,933,310 or 6,723,315.

Therapies that result in less muscle weakness and/or a smaller reduction in the number of motor neurons compared to the vehicle control in any of the above in vivo models of ALS are expected to be the most likely therapies to have a beneficial effect in humans for the treatment or prevention of ALS.

### Example 11

### Use of an In Vivo Model to Determine the Ability of Therapies of the Invention, Such as any of Therapies (1)-(7) to Treat, Prevent and/or Delay the Onset and/or the Development of a Neuronal Death Mediated Ocular Disease

In vivo models of ocular diseases can be used to determine the ability of any of the therapies described herein to treat and/or prevent and/or delay the onset and/or the development of a neuronal death mediated ocular disease.

One exemplary method for testing the activity of a therapy described herein to treat and/or prevent and/or delay the onset and/or development of a neuronal death mediated ocular disease such as macular degeneration, including the dry form of macular degeneration and/or Stargardt macular degeneration, employs the ELOVL4 mutant mouse model, as described by G. Karan et al. (Proc. Natl. Acad. Sci. USA, 2005, 102(11):4164-4169). This model involves transgenic mice expressing a mutant form of ELOVL4, which causes the mice to develop significant lipofuscin accumulation by the retinal pigment epithelium (RPE) followed by RPE death and photoreceptor degeneration. While mice apparently do not have maculas (the area within the central retina that is the most acutely involved with visual acuity), this model does cause degeneration and death of retinal cells in the center of the retina, similar to ARMD, and also causes retinal deposits that are very similar to the deposits (drusen) seen in ARMD. This model is believed to closely resemble human dry form macular degeneration and STGD.

In accordance with the method described by G. Karan (Proc. Natl. Acad. Sci. USA, 2005, 102(11):4164-4169), a 4-month experiment is conducted using 6 mice for high dose treatment, 6 mice for low dose treatment and 6 age-matched controls for non-treatment (weaning until 19 weeks). An average mouse is 20 g and drinks 15 ml/100 g body weight, or 3 ml per day. A high dose of a therapy of the invention is a therapy containing 36 μg/g per day, or 720 μg/mouse per day of a therapeutic compound. A low dose of a therapy is a therapy containing 12 μg/g per day, or 240 μg/mouse per day of a therapeutic compound. Drinking water therefore contains 240 μg/ml (high dose) and 80 μg/l (low dose) of a therapy. The exact amount of therapy consumed by each animal (housed in a separate cage) may be determined retrospectively. The neuroprotective effects of a therapy of the invention may also be tested in this protocol at lower doses, including 0.01 mg/kg, 0.05 mg/kg, 0.10 mg/kg, 1 mg/kg, and 5 mg/kg, or using other models of a neuronal death mediated ocular disease.

Analysis at the end of the 4 months of treatment is performed using histological sectioning and quantification of photoreceptor cell loss. Histological sectioning and quantification may be by the methods described by G. Karan et al. (Proc. Natl. Acad. Sci. USA, 2005, 102(11):4164-4169), such as those involving microscopy.

Other endpoints may be considered, such as: (1) body weights taken once weekly; (2) cageside clinical observations of the mice, such as once/daily to twice/weekly with observations recorded in a lab notebook; (3) collection and analysis of terminal plasma sample for each mouse, which sample may be kept in EDTA for pharmacokinetic or other analysis; (3) collection and analysis on water bottle samples taken from time to time to document that the therapy is stable during the period in which it is available to the mouse in the water (e.g., save a 0.5 to 1 mL sample, freeze at −80° C.).

### Example 12

### Method of Evaluating the NMDA-Induced Current Blocking Properties of Therapies of the Invention, Such as Therapies (1)-(7)

Therapies of the invention may be evaluated to determine their NMDA-induced current blocking properties. Experiments are carried out by the patch clamp method on freshly isolated neurons of a rat brain cortex or on cultured rat hippocampus neurons. Neurons for cultivation are obtained from the hippocampus of neonatal rats (1-2 days) by the method of trypsinization followed by pipetting. Cells suspended in culture medium are placed in 3 mL quantities into the wells of a 6-well planchette (Nunc) or into Petri dishes, in which glasses coated with poly-L-lysine has first been placed. The cell concentration is typically 2.5×10⁻⁶−5×10⁻⁶ cells/mL. The culture medium consists of Eagle's minimal medium and a DME/F12 medium (1:1) supplemented with 10% calf serum, 2 mM glutamine, 50 μg/ml gentamycin, 15 mM glucose, and 20 mM KCl, with the pH brought to 7-7.4 using NaHCO₃. Planchettes containing cultures are placed in a CO₂ incubator at 37° C. and 100% humidity. Cytosine arabinoside 10-20 μL is added on the second to third day of cultivation. After 6-7 days of cultivation, 1 mg/mL glucose is added to the medium, or the medium is exchanged, depending on the following experiment. The cultured hippocampal neurons are placed in a 0.4 mL working chamber. The working solution has the following composition: 150.0 mM NaCl, 5.0 mM KCl, 2.6 mM CaCl₂, 2.0 mM MgSO₄·7H₂O2.0, 10.0 mM HEPES, and 15.0 mM glucose, pH 7.36.

Transmembrane currents produced by application of NMDA are registered by the patch clamp electrophysiological method in the whole cell configuration. Application of substances is done by the method of rapid superfusion. Currents are registered with the aid of borosilicate microelectrodes (resistance 3.0-4.5 mOhm) filled with the following composition: 100.0 mM KCl, 11.0 mM EGTA, 1.0 mM CaCl₂ 1.0, 1.0 mM MgCl₂ 1.0, 10 mM HEPES, and 5.0 mM ATP, pH 7.2. An EPC-9 instrument (HEKA, Germany) is used for registration. Currents are recorded on the hard disk of a Pentium-IV PC using the pulse program, which is also purchased from HEKA. The results are analyzed with the aid of the Pulsefit program (HEKA).

Application of NMDA induces inflow currents in the cultured hippocampus neurons. Therapies of the invention that have a blocking effect on currents caused by the application of NMDA are expected to be useful as NMDA antagonists or as therapies that have one or more NMDA antagonist properties for the treatment of any of the diseases disclosed herein involving NMDA. Therapies can also be tested determine if they reduce the blocking effect of MK-801 on NMDA-induced currents in cultured rat hippocampus neurons. A reduction of the channel-blocking effect of MK-801 (and analogously phencyclidine) on NMDA receptors may lead to a decrease of their psychotomimetic effect and, therefore, to elimination of symptoms characteristic for schizophrenia. Thus, therapies of the invention that reduce the blocking effect of MK-801 are expected to be useful for treating, preventing and/or delaying the onset and/or the development of schizophrenia

### Example 13

### Use of an In Vivo Model to Determine the Ability of Therapies of the Invention, Such as any of Therapies (1)-(7) to Treat, Prevent and/or Delay the Onset and/or the Development of Schizophrenia

In vivo models of schizophrenia can be used to determine the ability of any of the therapies described herein to treat and/or prevent and/or delay the onset and/or the development of schizophrenia.

One exemplary model for testing the activity of one or more therapies described herein to treat and/or prevent and/or delay the onset and/or development of schizophrenia employs phencyclidene, which is chronically administered to the animal (e.g., non-primate (such as rat) or primate (such as monkey)), resulting in dysfunctions similar to those seen in schizophrenic humans. See Jentsch et al., 1997, Science 277:953-955 and Piercey et al., 1988, Life Sci. 43(4):375-385). Standard experimental protocols may be employed in this or other animal models. The neuroprotective effects of a therapy of the invention may also be tested in this protocol at doses including 0.01 mg/kg, 0.05 mg/kg, 0.10 mg/kg, 1 mg/kg, and 5 mg/kg, or using other models of schizophrenia.

### Example 14

### Determination of Calcium Blocking Properties of Therapies of the Invention, Such as any of Therapies (1)-(7)

Evaluation of the calcium-blocking properties of therapies of the invention is conducted with P2-fraction of synaptosomes, which are isolated from the brain of newborn (8-11 days) rats according to the protocol described by Bachurin et al. (“Neuroprotective and cognition enhancing properties of MK-801 flexible analogs. Structure-activity relationships,” Ann. N.Y. Acad. Sci., 2001, 939:219-235). In this assay, the ability of the therapies to inhibit a specific uptake of calcium ions via ion channels associated with glutamate receptors is determined.

Synaptosomes are placed into the incubation buffer A (132 mM NaCl, 5 mM KCl, 5 mM HEPES) and are kept at 0° C. during the entire experiment. Aliquots of synaptosomes (50 μl) are placed in medium A, containing therapies of the invention and a preparation of the radiolabeled calcium, ⁴⁵Ca. The calcium uptake is stimulated by the introduction into the medium of 20 μl of the 10 mM solution of glutamate. After a 5 minute incubation at 30° C., the reaction is interrupted by a filtration through GF/B filters, which are then triple-washed with cold buffer B (145 mM KCl, 10 mM Tris, 5 mM Trilon B). Then, filters are analyzed to detect radiolabeled calcium. The measurement is conducted using an SL-4000 liquid scintillation counter (Intertechnique, Fairfield, N.J., USA) The initial screening is conducted with a 5 μM solution of each compound. Specific calcium uptake is calculated using the following equation: K(43/21)=[(Ca₄−Ca₃)/(Ca₂−Ca₁)]*100%, where Ca₁ is calcium uptake in a control experiment (no glutamate or drug added); Ca₁ is calcium uptake in the presence of glutamate only (Glutamate Induced Calcium Uptake—GICU); Ca₃ is calcium uptake in the presence of a therapy only (no glutamate added); and Ca₄ is calcium uptake in the presence of both glutamate and therapy.

Therapies that possess pronounced calcium-blocking properties may have a potential as geroprotectors (Z. S. Khachaturian, “Calcium hypothesis of Alzheimer's disease and brain aging,” Ann. N.Y. Acad. Sci., 1994, 747:1-11).

### Example 15

### Determination of the Activity of Therapies of the Invention, Such as any of Therapies (1)-(7) as Geroprotectors

Therapies of the invention may be evaluated as agents that prolong life and/or improve the quality of life (characterized by changes in the amount or severity of pathologies that accompany aging) in the laboratory animals. Experiments are conducted with C57/B female mice, starting from the age of 12 months. Mice are kept in cells, 10 animals per cell. Both the control and experimental groups include 50 animals in each group. Animals have free access to food and water. The day-night cycle is 12 hours.

Prior to the experiment, daily and weekly water consumption by the animals in one cell is measured. In one aspect, a therapy of the invention is added in water in such amount that each animal consumes 3 mg/kg of the therapy per day in average. Bottles with water containing the therapy are replaced every 7 days. Animals in the control group receive pure water. Prior to the experiment, all the animals are weighed, and an average weight is determined in every group and in every cell, as well as the total weight of all animals in every cell. The condition of the skin, hair, and eyes are also determined by visual inspection. Preferably, all animals appear healthy and do not have any visible lesions prior to the experiment. Evaluation of all these parameters is conducted on a monthly basis. The neuroprotective effects of a therapy of the invention may also be tested in this protocol at lower doses, including 0.01 mg/kg, 0.05 mg/kg, 0.10 mg/kg, 1 mg/kg, and 5 mg/kg, or using other models of geroprotection.

### Lifespan

Length of life is evaluated using demographic methods. This parameter is a probability of death in every age group. Therapies that decrease or inhibit the probability of death are expected to be useful as geroprotectors.

### Dynamics in Weight of Animals

A decrease in the animal weight is expected during the experiment in the control group. This is a natural process, which is known as an age-related weight depletion. Therapies that decrease this weight loss are expected to be useful as geroprotectors.

### Vision Disturbances

Vision disturbances, appearing as a development of a cataract on one or both eyes, are expected in the control group of animals. Therapies that decrease the number of animals with cataracts are expected to be useful as geroprotectors.

### Skin and Hair Condition

Animals with disturbances in their skin-hair integument, in the form of bald spots or so-called alopecia, are expected in the control group. Therapies that decrease the number of animals with alopecia or the severity of alopecia are expected to be useful as geroprotectors.

### Example 16

### Determination of the Ability of Therapies of the Invention, Such as any of Therapies (1)-(7) to Inhibit Canine Cognitive Dysfunction Syndrome

The following exemplary experimental parameters can be used to test the ability of therapies of the invention to inhibit canine cognitive dysfunction syndrome in the following three examples (Examples 24-26). Therapies that result in an increase in activity (such as an increase in day time activity), an increase in locomotor activity, an increase in curiosity, or an increase in exploratory behavior are expected to be useful for inhibiting canine cognitive dysfunction syndrome (e.g., to cause a symptomatic improvement of age-associated behavioral deficits in dogs).

### Subjects

The exemplary subjects are summarized in Table 2. The only exclusion criteria is the absence of any disease or condition that could interfere with the purpose or conduct of the study.

### Summary of Subjects

| Species/breed: | Canine/Random Source Beagle Dogs |
|---|---|
| Initial age: | >7 years |
| Initial weight: | range from approximately 8 to 18 kg at study |
| | initiation |
| Sex: | both male and female |
| Origin: | Subjects are obtained from various sources |
| | and with the testing facility for at least 3 |
| | months |
| Identification: | Tattoo/Tags |
| Total: | 12 |

### Housing, Feeding and Environment

An exemplary test facility contains 2 areas for dog housing. The first consists of 32 stainless steel pens, in opposing rows of 16. Each pen is 5 feet×16 feet, with 2 foot×4 foot perches. Some of the pens are divided in half (2.5 feet×16 feet). The second consists of 24 galvanized steel pens in opposing rows of 12. In both areas, the floors are epoxy painted and heated. The exterior walls of the facility have windows near the ceiling (approximately 10 feet from ground level) that allow natural light to enter the facility. Dogs are housed generally four per cage based on compatibility and sex. A natural light-dark schedule is used. The pens are cleaned daily with a power washer.

Dogs are allowed free access to well water via a wall-mounted automatic watering system or in bowls. The dogs are fed a standard adult maintenance food (e.g., Purina Pro Plan® Chicken & Rice) once daily, with the amount adjusted to maintain a constant body weight.

Housing temperature and humidity is held relatively constant by automated temperature control and continuous ventilation. Room environmental conditions have design specifications as follows: single-pass air supply with a minimum of approximately 2100 c.f. filtered air changes per minute, relative humidity of 60±10%, temperature of 20±3° C., and a natural light-dark cycle.

Enrichment is provided by the presence of a pen mate and/or play toys. All dogs receive veterinary examinations prior to initiation in the study. Over the course of the study, trained personnel record all adverse events and contact the responsible veterinarian or study director when necessary.

### Dosing and Administration

Dogs are weighed prior to study initiation. Capsules containing a therapy of the invention are prepared for each dog according to weight. The following doses of a therapy of the invention may be used: 2, 6 and 20 mg/kg. The neuroprotective effects of a therapy of the invention may also be tested in this protocol at lower doses, including 0.01 mg/kg, 0.05 mg/kg, 0.10 mg/kg, 1 mg/kg, and 5 mg/kg, or using other models of canine cognitive dysfunction syndrome. Technicians not otherwise involved in the study prepare the capsules. During the control phase of the study, subjects are administered empty gelatin capsules. The test and control articles are administered to the dogs PO within meatballs of moist dog food once daily. Individual subjects are administered the capsule at the same time on each treatment day.

### Experimental Design

The design of the study consists of four 7 day test blocks (a test block refers to the 3 day washout period combined with the 4 day treatment/testing period). The first test block is a control and no subject receives treatment during those seven days. Subsequently, the study then follows a Latin-square design, in which all of the subjects are tested at all the three dose levels of the test article in a different order (see Table 3 below). To accomplish this, the twelve subjects are divided into six groups of two subjects balanced for sex and age to the extent possible.

Table 3. Canine Groups (groups A-F refer to canine groups that each have two dogs) and Dose Order (A in the Dose Order column refers to dose of 2 mg/kg; B in the Dose Order column refers to dose of 6 mg/kg and C in the Dose Order column refers to dose of 20 mg/kg).

| Canine Group | Dose Order |
|---|---|
| A | ABC |
| B | ACB |
| C | BAC |
| D | BCA |
| E | CAB |
| F | CBA |

After completing the control test block, each group receives three doses of the test article in the order prescribed for that group. For each test block, subjects receive their respective treatment for the first four days. On the fourth day of each test block, subjects are tested on the curiosity test twice; the first is one hour after article administration and the second is four hours after article administration. The remaining three days are considered washout days for each test block (Table 4).

### Subjects Received Four Days on Treatment and Three Washout Days During Each Test Block.

| Activity | Test Day(s) |
|---|---|
| Control | 1-4 |
| Wash 0 | 5-7 |
| Test Article Dose Phase 1 | 8-11 |
| Washout 1 | 12-14 |
| Test Article Dose Phase 2 | 15-18 |
| Washout 2 | 19-21 |
| Test Article Dose Phase 3 | 22-25 |

### Data Collection and Analysis

At the start of the study, an Actiwatch® collar is placed on each dog and the collar remains on for the duration of the study. All behavioral testing follows previously established protocols. For behavioral tests conducted in the open field arena, data analyses are conducted using the DogAct behavioral software (CanCog Technologies Inc., Toronto, ON, Canada). Actiware-Rhythm® software is used to obtain activity counts for the day-night measure.

The Actiwatch® data are analyzed to look at both changes in activity pattern temporally linked to treatment and changes in day/night activity.

To assess changes in activity linked to the treatment condition, hourly activity over a five hour period after dosing is calculated. The data are then analyzed with a repeated measures analysis of variance (ANOVA), with time post dosing (1-5 hours), treatment days (1-4 for each condition) and dose (control, 2, 6, and 20 mg/kg) as within subject variables. Test order serves as a between subject variable in the initial analysis. To examine day night-activity levels, day and night activity levels are calculated for each 24-hour period. The data are first analyzed with a repeated measures ANOVA, with dose (control, 2, 6, and 20 mg/kg), treatment day (1-4 for each condition), and phase (day and night) as within-subject variables. Once again order serves as a between-subject variable.

For the curiosity test, each behavioral measure is analyzed individually using a repeated measures ANOVA with dose (control, 2, 6, and 20 mg/kg), test (first and second) as within-subject variables and order as a between-subject variable.

All data are analyzed using the Statistica 6.0® software package (Statsoft, Inc., Tulsa, Okla., USA). Post-hoc Fisher's is used to examine main effects and interactions when appropriate.

### Post-Dose Activity Patterns and Day-Night Activity Rhythms

Activity is a marker associated with cognition. Activity is evaluated as a function of dose and time following treatment as well as a function of treatment day.

Post-dose activity patterns and twenty-four hour activity rhythms are assessed using the Actiwatch® method, which detects alterations in activity and changes in phase of the activity cycle as described previously (Siwak et al., 2003, “Circadian Activity Rhythms in Dogs Vary with Age and Cognitive Status,” Behav. Neurosci., 111:813-824). Briefly, general activity patterns are monitored for 28 continuous days using the Mini-Mitter® Actiwatch-16® activity monitoring system (Mini-Mitter Co., Inc., Bend, Oreg.) adapted for dogs. The Actiwatch-16® contains an activity sensor that is programmed to provide counts of total activity at 5 minute intervals. Putting the Actiwatch-16® on a dog's collar allows for recording uninterrupted patterns of activity and rest.

### Example 17

### General Activity Test to Determine the Ability of Therapies of the Invention to Inhibit Canine Cognitive Dysfunction Syndrome

The first analysis of the Actiwatch® data is intended to provide an overall picture of the post-dosing effect of the therapy on behavioral activity. Accordingly, data for the 5-hour period following dosing is first segregated into 5 one-hour blocks. Thus, each subject's data for each treatment day consists of 5 consecutive one-hour activity scores. The data are then analyzed with a repeated measures analysis of variance, with time post dosing (1-5 hours), treatment days (1-4 for each condition) and dose (control, 2, 6, and 20 mg/kg) as within subject variables. Test order serves as between subject variables in the initial analysis.

### Example 18

### Day Night Activity Assay to Determine the Ability of Therapies of the Invention to Inhibit Canine Cognitive Dysfunction Syndrome

The day/night activity data are analyzed with repeated-measures ANOVA, with dose, wash-in day, and phase as within-subject variables and test order as a between-subject variable.

### Example 19

### Curiosity Test to Determine the Ability of Therapies of the Invention to Inhibit Canine Cognitive Dysfunction Syndrome

This is a test of exploratory behavior, which assesses both attention to environment and locomotor activity (Siwak et al., 2001, “Effect of Age and Level of Cognitive Function on Spontaneous and Exploratory Behaviors in the Beagle Dog,” Learning Mem., 8:317-258). Subjects are placed in the open-field arena for a 10-minute period. Seven objects are placed in the arena and the subjects are permitted to freely explore the room and the objects.

The open field activity arena consists of an empty test room (approximately 8 feet×10 feet) with strips of electrical tape applied to the floor in a grid pattern of rectangles to facilitate tracking. The floor of the test room is mopped prior to testing and between dogs to reduce olfactory cues from affecting testing. For tests conducted in the open field, the dogs are placed in the test room and their behavior is videotaped over a 5- or 10-minute period. However, all dogs are tested on the control and 20 mg/kg dose and a separate analysis is carried out comparing control and high dose treatments.

The movement pattern of the dog within the test room is recorded. In addition, keyboard keys are pressed to indicate the frequency of occurrence of the various behaviors including: sniffing, urinating, grooming, jumping, rearing, inactivity and vocalization. The software also provides a total measure of distance for locomotor activity. In addition to general activity, the interactions with the objects (picking-up, contacting, sniffing and urinating on the objects) are assessed and used as measures of exploratory behavior. Urination frequency is indicative of marking behavior.

### Example 20

### Determination of the Ability of Therapies of the Invention, Such as any of Therapies (1)-(7) to Improve Cognitive Functions and Memory in an Animal Model

In order to study the action of therapies on the memory of animals in which there had been no prior destruction of neurons, a test of the recognition of the new location of a known object can be used (B. Kolb, K. Buhrmann, R. McDonald and R. Sutherland, “Object location memory test” Cereb. Cortex, 1994, 6:664-680; D. Gaffan, Eur. J. Neurosci., 1992, 4381-388; T. Steckler, W. H. I. M. Drinkenburgh, A. Sahgal and J. P. Aggleton, Prog. Neurobiol., 1998, 54:289-311).

Experiments are performed on C57BL/6 male mice aged 3-5 months and weighing 20-24 g. The animals are kept in a vivarium with 5 to a cage in 12/12 hours light/dark regime with light from 08.00 to 20.00 and free access to water and food. The observation chamber is made from white opaque organic glass and measures 48×38×30 cm. Brown glass vials with a diameter of 2.7 cm and a height of 5.5 cm are used as the test objects. 2-3 minutes before introducing an animal, the chamber and test objects are rubbed with 85% alcohol. The animals are always placed in the center of the chamber.

In one aspect, a therapy of the invention is dissolved in distilled water and administered intragastrically 1 hour before training in a volume of 0.05 ml per 10 g of animal weight. A corresponding volume of solvent is administered to control animals.

On the first day, the mice are brought into the test room and acclimatized for 20-30 minutes. After this, each animal is placed for 10 minutes in an empty behavior chamber, which has been pretreated with alcohol, for familiarization. The animal is then replaced in the cage and taken to the vivarium.

On the following day, the same mice are brought into the test room, acclimatized for 20-30 minutes, and then given the therapy (i.e., a solution containing a therapy of the invention) intragastrically. One hour after administration of the substance, an animal is placed in the behavior chamber on the bottom of which two identical objects for recognition (glass vials) are placed on a diagonal at a distance of 14.5 cm from the corners. The training time for each animal is 20 minutes. After 20 minutes, it is replaced in the cage and returned to the vivarium.

Testing is performed 48 hours after training. For this purpose, after acclimatization an animal is placed for 1 minute in the chamber for refamiliarization. After a minute, it is removed and one object is placed on the bottom of the chamber in a location known to the animal, and the other in a new location. The time spent investigating each object separately over a period of 10 minutes is recorded with an accuracy of 0.1 second using two electronic stopwatches. The behavior of the animals is observed through a mirror. Purposeful approach of an animal's nose towards an object at a distance of 2 cm or direct touching of an object with the nose is regarded as a positive investigative reaction.

The percent investigation time for each mouse can be calculated using the formula tN1/(tK1+tN1)×100. The total time spent on investigation of the two objects is taken as 100%. The results are further processed using the Student t-test method. Therapies that stimulate memory in this animal model are likely to do so in humans as well.

### Example 21

### Determination of the Ability of Therapies of the Invention, Such as any of Therapies (1)-(7) to Reduce Ischemic in a Rat Brain Model of Ischemia, Produced by Irreversible Occlusion of the Carotid Arteries

Therapies of the invention may also be tested to measure their ability to inhibit ischemia. Rat brain ischemia, produced by irreversible occlusion of the carotid arteries, is performed in accordance with methodological instructions for the experimental study of preparations for the treatment of cerebral circulation and migraine—“Handbook on the experimental (preclinical) study of new pharmacological substances”, Meditsina, Moscow, 2005, pp. 332-338.

Experiments are performed on cross-bred male white rats weighing 200-250 g, anesthetized with chloral hydrate (350 mg/kg, i/p). Irreversible single-step bilateral ligation of the common carotid arteries is performed on the animals. In the group of sham-operated animals, the ligatures are applied to the vessels but are not tightened. After completing the operation, the animals are divided randomly into groups: group one rats are given a therapy of the invention in a dose, e.g., of 0.1 mg/kg intraperitoneally after 30 minutes, then daily for 14 days after operation; group two rats are given nimodipine in a dose of 0.1 mg/kg intraperitoneally after 30 minutes, then daily for 14 days after operation. The neuroprotective effects of a therapy of the invention may also be tested in this protocol at lower doses, including 0.001 mg/kg, 0.005 mg/kg, 0.01 mg/kg, 0.05 mg/kg, 0.10 mg/kg, 1 mg/kg, and 5 mg/kg, or using other models of ischemia. Nimodipine is used to compare the effectiveness of a therapy of the invention. Control group and sham-operated animals are given physiological saline (0.9% sodium chloride) at the same times. The data are processed statistically with the aid of the Biostat program, using parametric and nonparametric methods.

The neurological deficit in animals with cerebral ischemia induced by ligation of the carotid arteries is determined using the McGraw Stroke-index in the modification of I. V. Gannushkina (Functional angioarchitectonics of the brain, Moscow, Meditsina, 1977, 224 pp). The severity of the condition is determined from the sum of the corresponding scores. The number of rats with mild symptoms up to 2.5 points on the Stroke-index scale (sluggish movements, limb weakness, hemiptosis, tremor, circular movements) and with severe manifestations of neurological impairment (from 3 to 10 points)—limb paresis, paralysis of lower limbs, lateral position, is noted. Therapies that reduce the amount of damage, the severity or number of symptoms, or the number of deaths from ischemia are expected to be useful in treating ischemia in humans.

### Example 22

### Determination of the Ability of Therapies of the Invention, Such as any of Therapies (1)-(7) to Reduce Damage in an Intracerebral Post-Traumatic Hematoma (Hemorrhagic Insult) Model

Therapies of the invention may also be tested to see if they have a protective effect in an intracerebral post-traumatic hematoma (hemorrhagic insult) model. The study is performed in accordance with the methodological instructions for the experimental study of preparations for the treatment of cerebral circulation and migraine—“Handbook on the experimental (preclinical) study of new pharmacological substances,” Meditsina, Moscow, 2005, pp. 332-338 in the modification of A. N. Makarenko et al. (Method for modeling local hemorrhage in various brain structures in experimental animals. Zh. vyssh. nervn. deyat., 2002, 52(6):765-768).

The experiments are performed on cross-bred male white rats weighing 200-250 g, kept in a vivarium with free access to food (standard pelleted feed) and water, and with natural alternation of day and night. Using a special device (mandrin-knife) and stereotaxis, brain tissue of rats anesthetized with nembutal (40 mg/kg, i/m) is destroyed in the region of the capsule interna, with subsequent (after 2-3 minutes) introduction into the damage site of blood taken from under the rat's tongue (0.02-0.03 ml). Scalping and trepanning of the skull are performed on sham-operated animals.

The animals are divided into 4 groups: sham-operated, a group of animals with hemorrhagic insult, animals with hemorrhagic insult which received a therapy of the invention in a dose of, e.g., 0.1 mg/kg, and animals with hemorrhagic insult which received nimodipine in a dose of 0.1 mg/kg. The neuroprotective effects of a therapy of the invention may also be tested in this protocol at lower doses, including 0.01 mg/kg, 0.05 mg/kg, 0.10 mg/kg, 1 mg/kg, and 5 mg/kg, or using other models of hemorrhagic insult. The effects of the substances are recorded 24 hours, and 3, 7 and 14 days after operation.

A therapy of the invention and nimodipine are administered to animals with insult in an identical dose of, e.g., 0.1 mg/kg intraperitoneally 3-3.5 hours after operation, and then daily for 14 days after operation. Physiological saline is administered to the control groups of animals. Each group consists of 9-18 animals at the start of the experiment.

The neurological deficit in the animals is determined using the McGraw Stroke-index in the modification of I. V. Gannushkina (Functional angioarchitectonics of the brain, Moscow, Meditsina, 1977, 224 pp). The severity of the condition is determined from the sum of the corresponding scores. The number of rats with mild symptoms up to 2.5 points on the Stroke-index scale (sluggish movements, limb weakness, unilateral hemiptosis, tremor, circular movements) and with severe manifestations of neurological impairment (from 3 to 10 points)—limb paresis, paralysis of lower limbs, lateral position, is noted. Rat deaths are recorded over the entire period of observation (14 days). The data are processed statistically with the aid of the Biostat program, using parametric and nonparametric methods. Nimodipine (in a dose of 0.1 mg/kg) is employed as the standard, using the scheme described above.

Therapies that reduce the amount of damage, the severity or number of symptoms, or the number of deaths from the hemorrhagic insult are expected to be useful in treating hemorrhagic insult in humans.

### Example 23

### Use of an In Vitro Model to Determine the Ability of Therapies of the Invention, Such as any of Therapies (1)-(7), to Treat, Prevent and/or Delay the Onset and/or the Development of MCI

In vivo models of MCI can also be used to determine the ability of any of the therapies described herein to treat, prevent and/or delay the onset and/or the development of MCI in mammals, such as humans. Several animal models of MCI have been developed by others.

For example, cognition and neuropathology in the aged-canine (dog) has been used by others as a model for MCI and AAMI (Cotman et al., Neurobiol. Aging., 2002, 23(5):809-18). Also, ischemia reperfusion injury models of brain hypoperfusion can be used. For example, the two-vessel carotid artery occlusion rat model, such as the 2-VO system, results in chronic brain hypoperfusion and mimics MCI and vascular changes in AD pathology (Obrenovich et al., Neurotox Res., 10(1):43-56, 2006). Similarly, De la Torre et al. (J. Cereb. Blood Flow Metab., 2005, 25(6):663-7) have reported an aging rat model of chronic brain hypoperfusion (CBH) that mimics MCI.

### Example 24

### Use of an In Vitro Model to Determine the Ability of Therapies of the Invention, Such as any of Therapies (1)-(7), to Treat, Prevent and/or Delay the Onset and/or the Development of AAMI

In vivo models of AAMI can also be used to determine the ability of any of the therapies described herein to treat, prevent and/or delay the onset and/or the development of AAMI in mammals, such as humans. Several animal models of AAMI have been developed by others. For example, as noted in the previous example, the canine represent a higher animal model to study the earliest declines in the cognitive continuum that includes AAMI and MCI observed in human aging (Cotman et al., Neurobiol Aging., 2002, 23(5):809-18).

### Example 25

### Use of Human Clinical Trials to Determine the Ability of Therapies of the Invention, Such as any of Therapies (1)-(7) to Treat, Prevent and/or Delay the Onset and/or the Development of a Disease or Condition for which the Activation, Differentiation, and/or Proliferation of One or More Cell Types is Beneficial

If desired, any of the therapies of the invention can also be tested in humans to determine the ability of the therapy to treat, prevent and/or delay the onset and/or the development of a disease or condition for which the activation, differentiation, and/or proliferation of one or more cell types is beneficial, such as a neurological indication described herein. Standard methods can be used for these clinical trials, such as those described in U.S. Pat. No. 5,527,814 or 5,780,489.

In one exemplary method, subjects with a disease or condition for which the activation, differentiation, and/or proliferation of one or more cell types is beneficial, are enrolled in a tolerability, pharmacokinetics and pharmacodynamics phase I study of a therapy using standard protocols such as those described in U.S. Pat. No. 5,780,489. Then a phase II, double-blind randomized controlled trial is performed to determine the efficacy of the therapy (see, for example, U.S. Pat. No. 5,780,489). If desired, the activity of the therapy can be compared to that of any other clinically used treatment for that disease or condition. Subjects may be analyzed for the progression of the disease or condition using standard methods, such as a functional rating score or analysis of specific symptoms. Also, where applicable, the length of survival can be compared between treatment groups (see, for example, U.S. Pat. No. 5,780,489).

### Example 26

### Randomized, Double Blinded, Placebo-Controlled Alzheimer's Disease Study

Exemplary human clinical trials for Alzheimer's disease are disclosed in U.S. Ser. No. 60/854,866, filed Oct. 27, 2006 (see, for example paragraphs [0144]-[0149]) and U.S. Pat. No. 7,071,206, issued Jul. 4, 2006. Briefly, patients with mild to moderate Alzheimer's disease (e.g., about 100 to 200 patients or any standard number of patients) are randomized to a therapy of the invention (e.g., 20 mg orally three times a day) or placebo for 6 months. Patients are evaluated with the ADAS-cog (primary endpoint), CIBIC-plus, MMSE, NPI and ADL at baseline, week 12 and week 26. The Alzheimer's Disease Assessment Scale—cognitive subscale (ADAS-cog) score assesses memory and cognition over time. The Mini Mental State Exam (MMSE) also assesses memory and cognition. The Alzheimer's Disease Cooperative Study-Clinical Global Impression of Change (ADCS-CGIC, also called CIBIC-plus) measures the patient's global status over time. It takes into account memory, cognition, behavior and motor disturbance. The Neuropsychiatric Inventory (NPI) measures the patients' behavior and psychiatric disturbance in 12 domains including delusions, hallucinations, agitation/aggression, depression/dysphoria, anxiety, elation/euphoria, apathy/indifference, disinhibitions, irritability/lability, motor disturbance, nighttime behaviors, and appetite/eating. ADAS-cog, CIBIC-plus, MMSE, NPI and ADL scores relative to placebo at week 26. Scales used to evaluate a therapy of the invention are known by those of skill in the art and are described, e.g., by Delegarza, V. W., 2003, Am. Fam. Phys., 68:1365-1372, and Tariot, P. N. et al., 2000, Neurol., 54:2269-2276.

Therapies of the invention that improve ADAS-cog, CIBIC-plus, MMSE, NPI and/or ADL scores are expected to be useful to treat, prevent and/or delay the onset and/or the development of Alzheimer's disease.

### Example 27

### Use of an In Vivo Model to Determine the Ability of Methods of the Invention to Treat Spinal Cord Injury

The ability of the methods of the invention to treat spinal cord injury is assessed in vivo using Wistar rats. In one study, the effect of a therapeutic hydrogenated pyrido[4,3-b]indole or pharmaceutically acceptable salt thereof, such as dimebon, to treat spinal cord injury is assessed.

Eight male and eight female rats aged two months and weighing between 250-300 g are divided into four groups, each containing two male and two female animals. Animals are housed on a 12 hour light/dark cycle with food and water freely available throughout, according to standard institutional and ethical protocols for the use of animals in laboratory experiments. After a 3 day acclimation period, the animals are administered a prophylactic dose of the antibiotic ciprofloxacin. Two hours later, the animals are anesthetized with a solution containing 20% chlorpromazine/80% ketamine administered via intramuscular injection. The animals are then positioned appropriately, disinfected, and a surgical spinal cord transection is performed between thoracic vertebrae 13 (T-13) and lumbar vertebrae 3 (L-3). On recovery, all animals are shown to have lost mobility below the level of the spinal cord transaction, with a full loss of spontaneous mobility in the lower paws and tail. Dimebon is diluted to the appropriate concentration in sterile saline solution. Animals in group 1 are given Dimebon at 10 mg/kg twice daily for eight weeks. Animals in group 2 are given Dimebon at 30 mg/kg twice daily for eight weeks. Animals in group 3 are given Dimebon at 60 mg/kg twice daily for eight weeks. Animals in group 4 are given an identical volume of vehicle (i.e., saline solution) twice daily for eight weeks. Spontaneous mobility in the lower paws and tail is tested in each animal weekly.

In a second study, the ability of administration of differentiated neurons produced by the ex vivo methods of the invention to treat spinal cord injury is assessed. Eight male and eight female rats aged two months and weighing between 250-300 g are divided into two groups, each containing four male and four female animals. Animals are housed on a 12 hour light/dark cycle with food and water freely available throughout, according to standard institutional and ethical protocols for the use of animals in laboratory experiments.

After a 3-day acclimation period, skin, bone marrow and plasma samples are taken from each animal, and multipotential stem cells (MSCs) isolated from each by standard methods. Cells are washed and triturated, then suspended in appropriate volume of Neurobasal medium supplemented with 2% B27 and 0.5 mM L-glutamine (all from GIBCO). Cells are plated to an appropriate density in wells on poly-L-lysine-coated plates and incubated at 37° C. in 5% CO₂-95% air atmosphere. After the MSCs have adhered to the plates and are growing normally, the cells are treated daily with an effective amount of 10 nM Dimebon in saline. Differentiation of the MSCs is monitored daily until more than 70% of cells observed in each well have sprouted neurites or shown other signs of differentiation. Cells are then washed with sterile Neurobasal medium, incubated with anti-NeuN antibody, which binds a neuron-specific antigen, and separated on a flow cytometer. Neurons are collected, washed to dissociate the antibody, and collected again in isotonic buffer for administration to paraplegic rats prepared as described above. One group of animals is treated with differentiated neurons, while the control group is treated with an equivalent volume of isotonic buffer. The differentiated neurons are implanted at the site of the spinal transection between T-13 and L-3. Spontaneous mobility in the lower paws and tail is tested in each animal each week for eight weeks. Any of the methods and combination therapies disclosed herein may be tested in this experimental model.

### Example 28

### Use of an In Vivo Model to Determine the Ability of the Methods of the Invention to Treat Experimental Autoimmune Encephalomyelitis (“EAE”)

Experimental Autoimmune Encephalomyelitis (“EAE”) is a well-established animal model for multiple sclerosis (“MS”) in humans. EAE is an acute or chronic-relapsing, acquired, inflammatory, demyelinating autoimmune disease acquired in animals by injection with proteins or protein fragments of various proteins that make up myelin, the insulating sheath that surrounds neurons. Proteins commonly used to induce EAE include myelin basic protein (MBP), proteolipid protein (PLP), and myelin oligodendrocyte glycoprotein (MOG). Those proteins induce an autoimmune response in the animals, resulting in an immune response directed to the animal's own myelin proteins that in turn produces a disease process closely resembling MS in humans.

EAE has been induced in a number of different animal species including mice, rats, guinea pigs, rabbits, macaques, rhesus monkeys and marmosets. For various reasons including the number of immunological tools, the availability, lifespan and fecundity of the animals and the resemblance of the induced disease to MS, mice and rats are the most commonly used species. In-bred strains are used to reliably produce animals susceptible to EAE. As with humans and MS, not all mice or rats will have a natural propensity to acquire EAE.

Eight male and eight female rats aged two months and weighing between 250-300 g are divided into two groups, each containing four male and four female animals. Animals are housed on a 12 hour light/dark cycle with food and water freely available throughout, according to standard institutional and ethical protocols for the use of animals in laboratory experiments. After a 3-day acclimation period, skin, bone marrow and plasma samples are taken from each animal, and multipotential stem cells (MSCs) isolated from each by standard methods. While the MSCs are being cultured and undergoing differentiation, each animal is injected with an amount of myelin basic protein (MBP) sufficient to induce EAE.

Cells are washed and triturated, then suspended in appropriate volume of Neurobasal medium supplemented with 2% B27 and 0.5 mM L-glutamine (all from GIBCO). Cells are plated to an appropriate density in wells on poly-L-lysine-coated plates and incubated at 37° C. in 5% CO₂-95% air atmosphere. After the MSCs have adhered to the plates and are growing normally, the cells are treated daily with an effective amount of 10 nM Dimebon in saline. Differentiation of the MSCs is monitored daily until more than 70% of cells observed in each well have sprouted neurites or shown other signs of differentiation. MSCs from a desired source (i.e., purified from skin, bone marrow or plasma) are then washed with sterile Neurobasal medium, incubated with anti-NeuN antibody, which binds a neuron-specific antigen, and separated on a flow cytometer. Neurons are collected, washed to dissociate the antibody, and collected again in isotonic buffer for administration to rats having EAE. One group is injected with differentiated neurons at an appropriate site, while the control group is injected with an equivalent volume of isotonic buffer at the same site used in Group I. Severity of EAE symptoms is evaluated weekly for four weeks according to standard clinical diagnostic criteria. Any of the methods and combination therapies disclosed herein may be tested in this experimental model.

### Example 29

### Dimebon Stabilizes Mitochondria to Calcium Overload with the Ionophore Ionomycin

Primary neuronal cultures were prepared from rat fetal tissue on the gestation days indicated from the cortex (day 17), hippocampus (day 19) or spine (day 15). Neurons were dissociated by trypsinization in the presence of DNAseI and trituration and cultured in Neurobasal (Gibco) medium supplemented with 2% B27 (Gibco), 0.5 mM L-Glutamine. Cells were plated on poly L-lysine coated plates, allowed to adhere and maintained at 37° C. in 5% CO₂-95% air and then test compound was added for a period of 3 days. Cultures were fixed using 2.5% glutaraldehyde. Approximately 80 digital images were taken per condition. Neurite length was calculated using Image-Pro Plus. Each analysis was performed with two separate culture studies. Dimebon was tested at concentrations of 0.01-500 nM and BDNF at 50 ng/mL. Mitochondrial effects were assessed with primary rat hippocampal cells treated with Dimebon or vehicle and 0.25 μM ionomycin. Dimebon's effects on JC-1 mitochondrial staining was also assessed in ionomycin-treated human neuroblastoma cells (SK—N—SH). Mitochondrial accumulation of JC-1 was assessed by fluorescence microscopy.

Using primary rat hippocampal cells treated with ionomycin Dimebon (0.25 nM and 2.5 nM) preserved mitochondrial JC-1 staining compared with vehicle treatment. Similarly, Dimebon treatment preserved mitochondrial JC-1 staining in ionomycin treated SK—N—SH cells. Dimebon stabilizes mitochondria to calcium overload with the ionophore ionomycin suggesting the compound prevents the loss of mitochondrial membrane polarity.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

All references, publications, patents, and patent applications disclosed herein are hereby incorporated by reference in their entirety.

1. A method of:
(a) treating delaying the onset, and/or delaying the development of a condition; or
(b) stimulating neurite outgrowth and/or enhancing neurogenesis in an individual in need thereof;
wherein the method of (b) comprises administering to the individual an effective amount of a first therapy comprising a hydrogenated pyrido[4,3-b]indole of the formula (A) or (B): 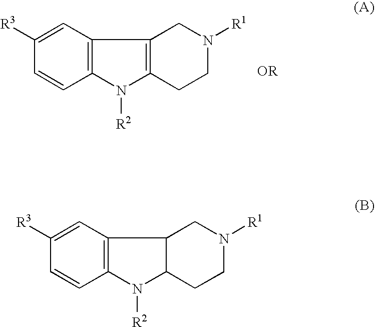
wherein:
R¹ is lower alkyl or aralkyl;
R² is hydrogen, aralkyl or substituted heteroaralkyl; and
R³ is selected from hydrogen, lower alkyl or halo
or pharmaceutically acceptable salt thereof,
and wherein the method of (a) comprises either: (1) administering to the individual an effective amount of a first therapy comprising a hydrogenated pyrido[4,3-b]indole of the formula (A) or (B), or a pharmaceutically acceptable salt thereof or (2) administering to the individual an effective amount of a cell that has been incubated with a hydrogenated pyrido[4,3-b]indole of the formula (A) or (B), or pharmaceutically acceptable salt thereof, in an amount and under conditions sufficient to activate the cell, promote the differentiation of the cell, promote the proliferation of the cell, or any combination of two or more of the foregoing, and
wherein the individual has a condition selected from the group consisting of injury-related mild cognitive impairment (MCI), neuronal death mediated ocular disease, macular degeneration, autism, autism spectrum disorder, Asperger syndrome, Rett syndrome, an avulsion injury, a spinal cord injury, myasthenia gravis, Guillain-Barre syndrome, multiple sclerosis, neuropathy and a non-neuronal indication.

2. The method of claim 1, wherein the condition is one for which the activation, differentiation, and/or proliferation of one or more cell types is beneficial for treating, preventing, delaying the onset, and/or delaying the development of the condition.

3. A method of either:
(a) promoting the differentiation and/or proliferation of a cell; or
(b) differentiating multipotential stem cells
comprising incubing the cell with an amount of a hydrogenated pyrido[4,3-b]indole of the formula (A) or (B): 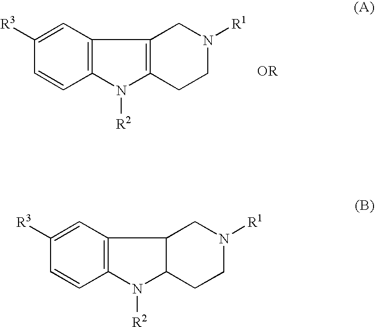
wherein:
R¹ is lower alkyl or aralkyl;
R² is hydrogen, aralkyl or substituted heteroaralkyl; and
R³ is selected from hydrogen, lower alkyl or halo,
or pharmaceutically acceptable salt thereof, and under conditions sufficient to promote the differentiation and/or proliferation of the cell.

4. The method of claim 3, wherein the cell is a neuronal cell and the differentiation and/or proliferation comprises stimulating neurite outgrowth and/or enhancing neurogenesis of the cell.

5. (canceled)

6. A method of treating, delaying the onset, and/or delaying the development of a condition for which the activation, differentiation, and/or proliferation of one or more cell types is beneficial in an individual in need thereof, the method comprising either:
(a) administering to the individual an effective amount of a combination of (i) a first therapy comprising a hydrogenated pyrido[4,3-b]indole of the formula (A) or (B): 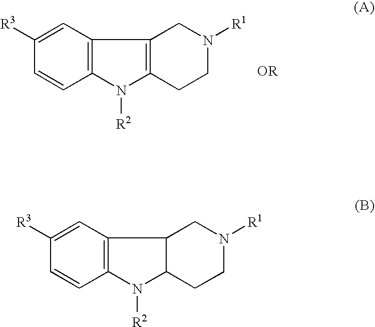
wherein:
R¹ is lower alkyl or aralkyl;
R² is hydrogen, aralkyl or substituted heteroaralkyl; and
R³ is selected from hydrogen, lower alkyl or halo,
or pharmaceutically acceptable salt of any of the foregoing and (ii) a second therapy comprising a growth factor and/or anti-cell death compound; or
(b) administering to the individual an effective amount of a combination of (i) a first therapy comprising a hydrogenated pyrido[4,3-b]indole of the formula (A) or (B) or pharmaceutically acceptable salt thereof and (ii) a second therapy comprising a cell.

7-10. (canceled)

11. A method of aiding in the treatment of an individual having a neuronal indication or non-neuronal indication comprising administering to the individual differentiated cells produced by the method of claim 3.

12. The method of any one of claims 1, 3 and 6, wherein the hydrogenated pyrido[4,3-b]indole is 2,8-dimethyl-5-(2-(6-methyl-3-pyridyl)ethyl)-2,3,4,5-tetrahydro-1H-pyrido[4,3-b]indole, or a pharmaceutically acceptable salt thereof.

13-15. (canceled)

16. The method of claim 3 further comprising incubating the cell with a growth factor and/or an anti-cell death compound.

17. The method of any one of claims 1, 3 and 6, wherein the cell type is selected from the group consisting of multipotential stem cells, neuronal stem cells, non-neuronal cells, and neurons.

18. The method of any one of claims 1, 3 and 6, wherein the cell type is a neuron, and wherein the method increases the length of one or more axons of the neuron.

19. The method of any one of claims 1, 3 and 6, wherein the cell type is a neuronal stem cell, and wherein the method promotes differentiation of the neuronal stem cell into a neuron.

20. The method of claim 19, wherein the neuronal stem cell differentiates into a hippocampal neuron, a cortical neuron, or a spinal motor neuron.

21. The method of claim 6, wherein the first and second therapies are administered sequentially.

22. The method of claim 6, wherein the first and second therapies are administered simultaneously.

23. The method of claim 6, wherein the first and second therapies are contained in the same pharmaceutical composition.

24. The method of claim 6, wherein the first and second therapies are contained in separate pharmaceutical compositions.

25-28. (canceled)

29. The method of claim 17, wherein the multipotential stem cell is either: (a) a neuronal stem cell that differentiates into hippocampal neurons, cortical neurons, or spinal motor neurons or (b) a non-neuronal stem cell that differentiates into a skin cell, a cardiac muscle cell, a skeletal muscle cell, a liver cell, a kidney cell, or a cartilage cell.

30. The method of claim 3, wherein the incubation occurs ex vivo.

31. The method of claim 3, wherein the incubation occurs in vivo.

32. The method of claim 3, further comprising the step of selecting a differentiated cell type from culture.

33. The method of claim 32, wherein the selected differentiated cell type is a hippocampal neuron, a cortical neuron, or a spinal motor neuron.

34. The method of claim 11, wherein the differentiated cells are either (a) neuronal cells selected from hippocampal neurons, cortical neurons, and spinal motor neurons; or (b) non-neuronal cells selected from skin cells, cardiac muscle cells, liver cells, kidney cells, and cartilage cells.

35-36. (canceled)

37. The method of claim 11, wherein the differentiated cells are administered systemically by intravenous injection.

38. The method of claim 11, wherein the differentiated cells are administered locally by direct injection or surgical implantation.

39. The method of claim 33, further comprising the step of administering the differentiated cells systemically by intravenous injection.

40. The method of claim 33, further comprising the step of administering the differentiated cells locally by direct injection or surgical implantation.

41. A pharmaceutical composition comprising
(a) a first therapy comprising:
(1) a hydrogenated pyrido[4,3-b]indole of the formula (A) or (B): 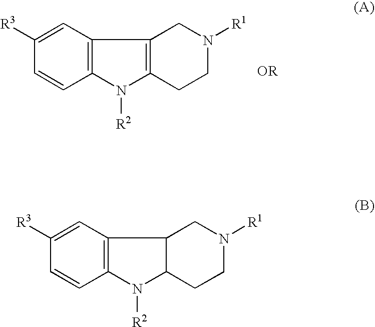
wherein:
R¹ is lower alkyl or aralkyl;
R² is hydrogen, aralkyl or substituted heteroaralkyl; and
R³ is selected from hydrogen, lower alkyl or halo,
or pharmaceutically acceptable salt in an amount sufficient to activate a cell, promote the differentiation of a cell, promote the proliferation of a cell, or any combination of two or more of the foregoing and/or
(2) a cell that has been incubated with a hydrogenated pyrido[4,3-b]indole of the formula (A) or (B), or pharmaceutically acceptable salt thereof, under conditions sufficient to activate the cell, promote the differentiation of the cell, promote the proliferation of the cell, or any combination of two or more of the foregoing; and
and (b) a pharmaceutically acceptable carrier.

42-43. (canceled)

44. A kit comprising:
(a) a first therapy comprising:
(1) a hydrogenated pyrido[4,3-b]indole of the formula (A) or (B): 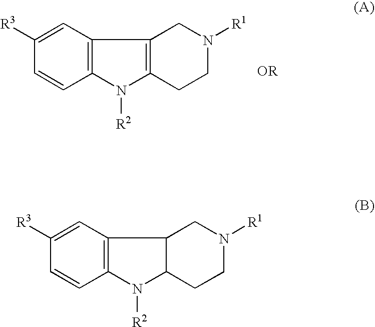
wherein:
R¹ is lower alkyl or aralkyl;
R² is hydrogen, aralkyl or substituted heteroaralkyl; and
R³ is selected from hydrogen, lower alkyl or halo,
or pharmaceutically acceptable salt thereof, in an amount sufficient to activate a cell, promote the differentiation of a cell, promote the proliferation of a cell, or any combination of two or more of the foregoing and/or
(2) a cell that has been incubated with a hydrogenated pyrido[4,3-b]indole of the formula (A) or (B), or pharmaceutically acceptable salt thereof, under conditions sufficient to activate the cell, promote the differentiation of the cell, promote the proliferation of the cell, or any combination of two or more of the foregoing; and
(b) instructions for use of in the treatment, prevention, slowing the progression, delaying the onset, and/or delaying the development of a condition for which the activation, differentiation, and/or proliferation of one or more cell types is beneficial.

45-46. (canceled)

47. The method of any of claims 1, 3 and 6 wherein the hydrogenated pyrido[4,3-b]indole is of the formula (A), or a pharmaceutically acceptable salt thereof.

48. The pharmaceutical composition of claim 41 or the kit of claim 44 wherein the hydrogenated pyrido[4,3-b]indole is of the formula (A), or a pharmaceutically acceptable salt thereof.

49. The method of any of claims 1, 3 and 6, wherein the pyrido[4,3-b]indole is of the formula (A), or a pharmaceutically acceptable salt thereof, wherein R¹ is CH₃—, CH₃CH₂—, or PhCH₂—; R² is selected from H—, PhCH₂—, or 6-CH₃-3-Py-(CH₂)₂—; and R³ is H—, CH₃— or Br—.

50. The pharmaceutical composition of claim 41 or the kit of claim 44 wherein the hydrogenated pyrido[4,3-b]indole is of the formula (A), or a pharmaceutically acceptable salt thereof, wherein R¹ is CH₃—, CH₃CH₂—, or PhCH₂—; R² is selected from H—, PhCH₂—, or 6-CH₃-3-Py-(CH₂)₂—; and R³ is H—, CH₃— or Br—.

51. The pharmaceutical composition of claim 41 or the kit of claim 44 wherein the hydrogenated pyrido[4,3-b]indole is 2,8-dimethyl-5-(2-(6-methyl-3-pyridyl)ethyl)-2,3,4,5-tetrahydro-1H-pyrido[4,3-b]indole, or a pharmaceutically acceptable salt thereof.

52. The method of claim 12 wherein the pyrido[4,3-b]indole is an acid salt of 2,8-dimethyl-5-(2-(6-methyl-3-pyridyl)ethyl)-2,3,4,5-tetrahydro-1H-pyrido[4,3-b]indole.

53. The pharmaceutical composition or the kit of claim 51 wherein the hydrogenated pyrido[4,3-b]indole is an acid salt of 2,8-dimethyl-5-(2-(6-methyl-3-pyridyl)ethyl)-2,3,4,5-tetrahydro-1H-pyrido[4,3-b]indole.

54. The method of claim 12 wherein the pyrido[4,3-b]indole is the dihydrochloride salt of 2,8-dimethyl-5-(2-(6-methyl-3-pyridyl)ethyl)-2,3,4,5-tetrahydro-1H-pyrido[4,3-b]indole.

55. The pharmaceutical composition or the kit of claim 51 wherein the hydrogenated pyrido[4,3-b]indole is the dihydrochloride salt of 2,8-dimethyl-5-(2-(6-methyl-3-pyridyl)ethyl)-2,3,4,5-tetrahydro-1H-pyrido[4,3-h]indole.
